(19) United States
(12) Patent Application Publication
(10) Pub. No.: US 2024/0409646 A1
(43) Pub. Date: Dec. 12, 2024

(54) METHODS FOR THE TREATMENT AND PREVENTION OF GPP

(71) Applicant: Boehringer Ingelheim International GmbH, Ingelheim am Rhein (DE)

(72) Inventors: Christian THOMA, Biberach an der Riss (DE); Denis DELIC, Biberach an der Riss (DE); Ahmed Karim FARAG, Biberach an der Riss (DE); Marcel ROLSER, Mietingen (DE); Sudha VISVANATHAN, Mount Kisco, NY (US)

(21) Appl. No.: 18/736,586

(22) Filed: Jun. 7, 2024

Related U.S. Application Data
(60) Provisional application No. 63/610,005, filed on Dec. 14, 2023.
(60) Provisional application No. 63/507,133, filed on Jun. 9, 2023.

(51) Int. Cl. C07K 16/28; A61K 9/00; A61K 39/00; A61P 17/06; C12Q 1/6883

(52) U.S. Cl. CPC C07K 16/2866; A61K 9/0019; A61P 17/06; C12Q 1/6883; A61K 2039/505; A61K 2039/54; A61K 2039/545; C07K 2317/565; C12Q 2600/106

(57) ABSTRACT

The present invention relates to the treatment of generalized pustular psoriasis (GPP), including the treatment and prevention of flares in adults and adolescents from 12 years of age with a history of generalized pustular psoriasis (GPP) with anti-IL36R antibodies.

### SEQUENCE LISTING

The instant application contains a Sequence Listing which has been submitted in XML format via Patent Center and is hereby incorporated by reference in its entirety. Said XML copy, created on Mar. 27, 2024, is named 09-0745-US-3_SL.XML and is 139,264 bytes in size.

### TECHNICAL FIELD OF THE INVENTION

The present invention relates to use of anti-IL-36R antibodies in methods and compositions for treatment of adults and adolescents from 12 years of age with a history of generalized pustular psoriasis (GPP) and/or as diagnosed per European Rare and Severe Psoriasis Expert Network (ERASPEN) criteria. More specifically, the invention relates to the treatment of generalized pustular psoriasis (GPP), including the treatment and prevention of flares in a subject by administering to the subject a subcutaneous loading dose of 300 or 600 mg of an anti-IL-36R antibody, followed by maintenance doses of 150 or 300 mg of the anti-IL-36R antibody administered subcutaneously at 4 or 12-week intervals.

### BACKGROUND

GPP is a rare systemic auto inflammatory skin disease, that presents as recurring episodes of severe flares affecting the skin and internal organs (Onoufriadis A, Simpson M A, Pink A E, et al. Mutations in IL36RN/IL1F5 are associated with the severe episodic inflammatory skin disease known as generalized pustular psoriasis. Am J Hum Genet 2011; 89(3):432-437; Choon S E, Lai N M, Mohammad N A, Nanu N M, Tey K E, Chew S F. Clinical profile, morbidity, and outcome of adult-onset generalized pustular psoriasis: analysis of 102 cases seen in a tertiary hospital in Johor, Malaysia. Int J Dermatol. 2014; 53(6):676-84)) which can lead to potentially life-threatening consequences. If left untreated, serious complications can arise such as infection and sepsis, resulting in hospitalization and even death, most commonly due to septic shock or cardiac or renal failure.

Acute GPP flares of varying severity occur in most subjects and may be idiopathic or triggered by external stimuli, such as infection, corticosteroid use or withdrawal, stress, or pregnancy. Moderate or severe GPP flares cause significant morbidity and mortality due to tender, painful skin lesions, extreme fatigue, high fever, peripheral blood neutrophilia and acute phase response and sepsis. The acute phase is associated with a mean duration of hospitalization of 10 days (range 3-44 days). The observed mortality rate of 7% reported in a retrospective study with 102 GPP cases seen in a tertiary hospital in Johor, Malaysia is likely an underestimate as not all GPP subjects were included in the study. Mortality rates are also likely underestimated due to lack of identifying the cause of death as GPP and are largely driven by infectious complications and extra-cutaneous organ manifestations such as renal, hepatic, respiratory and cardiac failure.

After responding to treatment or spontaneous flare cessation, it is estimated that up to 50% of subjects may suffer from chronic GPP characterized by persistent erythema and scaling that may also include joint symptoms.

The classic presentation of GPP flares as described by von Zumbusch is strongly correlated with polymorphisms in the IL36-R signaling pathway. Individuals with loss-of-function mutations of the IL36RN gene which encodes an endogenous IL36R antagonist (IL-36RN) have dramatically higher incidence of GPP, indicating that uncontrolled upregulation of IL36 signaling due to defective IL36RN antagonism leads to the inflammatory episodes observed in GPP. Genetic human studies have demonstrated the occurrence of GPP clusters in families with a loss of function mutation in IL36RN, which results in uncontrolled IL36R signaling. Mutations in other genes linked to the IL36 pathway such as CARD14 also lead to GPP. A recently published gene expression study indicates sustained activation of IL-1 and IL-36 in GPP, inducing neutrophil chemokine expression, infiltration, and pustule formation, suggesting that the IL-1/IL-36 inflammatory axis is a potent driver of disease pathology in GPP. Moreover, a recent meta-analysis investigated 233 published GPP cases. They found that 49 (21.0%) of 233 cases carried recessive IL36RN alleles. Those 49 recessive IL36RN alleles defined a GPP phenotype characterized by early onset and high risk of systemic inflammation.

IL36R is a cell surface receptor involved in inflammatory responses in skin and gut. It is a novel member of the IL1R family that forms a heterodimeric complex with the IL1R accessory protein. The heterodimeric IL36R system with stimulating (IL36a, IL36p, IL36γ) and inhibitory ligands (IL36Ra) shares a number of structural and functional similarities to other members of the IL1/IL1R family, such as IL1, IL18 and IL33 (R17-3602). All IL1 family members (IL1α, IL1β, IL18, IL36α, IL36β, IL36γ, and IL38) signal through a unique, cognate receptor protein which, upon ligand binding, recruits the common IL1 RacP subunit and activates NFkB and MAP kinase pathways in receptor-positive cell types. In human skin tissues, IL36R is expressed in keratinocytes, dermal fibroblasts and infiltrating myeloid cells. IL36R activation in skin tissue drives the production of inflammatory mediators (e.g., CCL20, MIP-1β, TNF-α, IL12, IL17, IL23, TGF-β) and modulates the expression of tissue remodeling genes (e.g., MMPs, TGF-β). Therefore, the link between GPP and mutations in the IL36RN is somewhat analogous to the well-established neonatal onset of sterile multifocal osteomyelitis, periostitis, and pustulosis caused by absence of interleukin-1-receptor antagonist. In this case, absence of the receptor antagonist allows unopposed action of interleukin-1, resulting in life-threatening systemic inflammation with skin and bone involvement. These clinical features responded to empirical treatment with the recombinant interleukin-1-receptor antagonist anakinra.

To adequately treat GPP, both the acute phase of disease as well as the long-term chronic diseases need to be treated, that is, acute flares must be addressed as of when they spontaneously occur, and maintenance therapy must be administered to prevent future flares. Treatment and prevention of GPP flares aims to control the serious acute condition by controlling the systemic signs (CRP, fever, neutrophilia) and pustulation (e.g., visible sign of the inflammation), and further to prevent infection and onset of other complications that may make the event life threatening. Long term maintenance treatment however is the key to the sustained control of the signs and symptoms of the GPP flare and the prevention of the occurrence of flare.

The European Rare And Severe Psoriasis Expert Network (ERASPEN) consensus criteria include as key diagnosis criteria for acute GPP, the presence of primary, sterile, macroscopically visible pustules on non-acral skin (excluding cases where pustulation was restricted to psoriatic plaques), with or without systemic inflammation, with or without plaque-type psoriasis, either relapsing (>1 episode) or persistent (>3 months) (Website:eraspen.eu/home/rfp/diagnostic-criteria.html (access date: 9 May 2018); European Rare And Severe Psoriasis Expert Network (ERASPEN); 2018).

Chronic GPP describes the state between disease flares that may be characterized by the persistence of residual skin symptoms such as erythema and scaling and pustulation. The clinical presentation of GPP is in episodic nature, that may include with normal appearing skin during the chronic phase between very acute and severe disease flares.

Although approved GPP flare treatments are available, there is still the unmet need for the treatment and prevention of GPP flares (Choon, et al. 2014, Id). Preventing GPP flares with a safe and effective treatment will meet a high unmet need to have a proven treatment available for subjects with frequent recurrence of this disruptive condition with high morbidity and associated mortality. Moreover, due to its symptom burden and severity, associated comorbidities, and scarcity of tailored treatments, GPP can have a profound impact on the person's health-related quality of life ever greater than other chronic skin conditions such as plaque psoriasis (psoriasis vulgaris, PV) (Lebwohl M, Medeiros R A, Mackey R H, et al. The Disease Burden of Generalized Pustular Psoriasis: Real-World Evidence From CorEvitas' Psoriasis Registry. Journal of Psoriasis and Psoriatic Arthritis 2022; 7(2): 71-8) Thus, a need exists in the art for novel targeted therapies for the treatment and/or prevention of GPP.

### SUMMARY OF THE INVENTION

The present invention addresses the above need by providing biotherapeutics, in particular antibodies, which bind to IL-36R and provide therapeutic and maintenance treatment of generalized pustular psoriasis (GPP) including the treatment and prevention of flares, by preventing the incidence of and/or frequency of GPP flares as well as other associated signs and symptoms of GPP flares.

In a first aspect, the present invention relates to the treatment of generalized pustular psoriasis (GPP), including the treatment and prevention of flares in adults and adolescents from 12 years of age with a history of generalized pustular psoriasis (GPP) and/or as diagnosed per European Rare And Severe Psoriasis Expert Network (ERASPEN) criteria, comprising administering or having administered to the adult and or adolescent a therapeutically effective amount of an anti-IL-36R antibody or an antigen-binding fragment thereof. In a preferred embodiment, the invention relates to the treatment of GPP in adults and adolescents from 12 years of age when not experiencing flare. In an embodiment relating to this aspect, the anti-IL-36R antibody is spesolimab.

In a second aspect, the present invention relates to a method of reducing or alleviating signs and symptoms of GPP in adults and adolescents from 12 years of age with a history of generalized pustular psoriasis (GPP) and/or as diagnosed per European Rare And Severe Psoriasis Expert Network (ERASPEN) criteria, said method comprising administering or having administered to the adult or adolescent a therapeutically effective amount of an anti-IL-36R antibody or an antigen-binding fragment thereof. In an embodiment relating to this aspect, the invention is a method of reducing or alleviating the number, prevalence of, severity of, and/or reoccurrence of flares of an adult or adolescent with GPP, said method comprising administering or having administered to the subject a therapeutically effective amount of an anti-IL-36R antibody or an antigen-binding fragment thereof. In an embodiment relating to this aspect, the anti-IL-36R antibody is spesolimab.

In a third aspect, the present invention relates to a method of reducing the occurrence of and/or frequency an acute flare of GPP in adults and adolescents from 12 years of age with a history of generalized pustular psoriasis (GPP) and/or as diagnosed per European Rare And Severe Psoriasis Expert Network (ERASPEN) criteria, said method comprising administering or having administered to the adult or adolescent a therapeutically effective amount of an anti-IL-36R antibody of the present invention or an antigen binding fragment thereof. In an embodiment relating to this aspect, the anti-IL-36R antibody is spesolimab.

In an embodiment relating to aspects one to three, the anti-IL-36R antibody or antigen-binding fragment thereof comprises: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62,108, 109, 110 or 111 (H-CDR2); the amino acid sequence of SEQ ID NO: 72 (H-CDR3).

In a further embodiment relating to aspects one to three, the anti-IL-36R antibody, or antigen-binding fragment thereof comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

In a further embodiment relating to aspects one to three, the anti-IL-36R antibody or antigen-binding fragment thereof comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

In an embodiment relating to aspects one to three, the anti-IL-36R antibody comprises or antigen-binding fragment thereof:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In an embodiment relating to any of the above aspects or embodiments, the anti-IL-36R antibody is spesolimab.

In an embodiment relating to any of the aspects one to three, a second therapeutic agent is administered to the subject before, after, or concurrent with the anti-IL-36R antibody or an antigen-binding fragment thereof. In a related embodiment, the second therapeutic agent is selected from the group consisting of an anti-bacterial agent, an anti-viral agent, an anti-fungal agent, another IL-36R antagonist, an anti PDE4, an IL-17 antagonist, an IL-12/IL-23 antagonist, an IL-23 antagonist, and IL-1 antagonist, an IgE inhibitor, a corticosteroid, NSAID, an IL-4R antagonist, a TNF-α inhibitor, and IFNγ.

Another embodiment of the invention relates to the treatment of generalized pustular psoriasis (GPP), including the treatment and prevention of flares in a subject with a history of GPP symptoms, and/or with a confirmed history (diagnosis) of GPP based on the consensus diagnostic criteria defined by the ERASPEN Diagnostic Criteria by administering to the subject a loading dose of 600 mg of an anti-IL-36R antibody or antigen-binding fragment thereof at week 0 or 1; followed by maintenance doses comprising 600 mg of said anti-IL-36R antibody administered to the subject at 2-, 4-, 5-, 6-, 7-, 8-, 10- or 12-week intervals. In a preferred embodiment, the invention relates to said loading and maintenance doses that are administered subcutaneously for the treatment of GPP in adults and adolescents from 12 years of age when not experiencing flare. In an exemplary embodiment, a subcutaneous loading dose of 600 mg (four 150 mg injections), are followed by 600 mg (four 150 mg injections) subcutaneously 4 weeks later and every 4 weeks thereafter.

Another embodiment of the invention relates to the treatment of generalized pustular psoriasis (GPP), including the treatment and prevention of flares in a subject with a history of GPP symptoms, and/or with a confirmed history (diagnosis) of GPP based on the consensus diagnostic criteria defined by the ERASPEN Diagnostic Criteria by administering to the subject a loading dose of 600 mg of an anti-IL-36R antibody or antigen-binding fragment thereof at week 0 or 1; followed by maintenance doses comprising 300 mg of said anti-IL-36R antibody administered to the subject at 2-, 4-, 5-, 6-, 7-, 8-, 10- or 12-week intervals. In a preferred embodiment, the invention relates to said loading and maintenance doses that are administered subcutaneously for the treatment of GPP in adults and adolescents from 12 years of age when not experiencing flare. In an exemplary embodiment, a subcutaneous loading dose of 600 mg (four 150 mg injections), are followed by 300 mg (two 150 mg injections) subcutaneously 4 weeks later and every 4 weeks thereafter.

In another aspect, the invention relates to treatment of generalized pustular psoriasis (GPP), including the treatment and prevention of flares in a subject with a history of GPP symptoms, and/or with a confirmed history (diagnosis) of GPP based on the consensus diagnostic criteria defined by the ERASPEN Diagnostic Criteria by administering to the subject a loading dose of 600 mg of an anti-IL-36R antibody or antigen-binding fragment thereof at week 0 or 1, followed by maintenance doses comprising 150 mg of said anti-IL-36R antibody administered to the subject at 2-, 4-, 5-, 6-, 7-, 8-, 10-, or 12-week intervals. In a preferred embodiment, the invention relates to said loading and maintenance doses that are administered subcutaneously for the treatment of GPP in adults and adolescents from 12 years of age when not experiencing flare. In an exemplary embodiment, a subcutaneous loading dose of 600 mg (four 150 mg injections), are followed by 150 mg (one 150 mg injections) subcutaneously 4 weeks later and every 4 weeks thereafter.

In another aspect, the invention relates to the treatment of generalized pustular psoriasis (GPP), including the treatment and prevention of flares in a subject with a history of GPP symptoms, and/or with a confirmed history (diagnosis) of GPP based on the consensus diagnostic criteria defined by the ERASPEN Diagnostic Criteria, by administering to the subject a loading dose of 300 mg of an anti-IL-36R antibody or antigen-binding fragment thereof at week 0 or 1, followed by maintenance doses comprising 300 mg of said anti-IL-36R antibody administered at 2-, 4-, 5-, 6-, 7-, 8-, 10-, or 12-week intervals. In a preferred embodiment, the invention relates to said loading and maintenance doses that are administered subcutaneously for the treatment of GPP in adults and adolescents from 12 years of age when not experiencing flare. In an exemplary embodiment, a subcutaneous loading dose of 300 mg (two 150 mg injections), are followed by 300 mg (two 150 mg injections) subcutaneously 4 weeks later and every 4 weeks thereafter.

In another aspect, the invention relates to the treatment of generalized pustular psoriasis (GPP), including the treatment and prevention of flares in a subject with a history of GPP symptoms, and/or with a confirmed history (diagnosis) of GPP based on the consensus diagnostic criteria defined by the ERASPEN Diagnostic Criteria, by administering to the subject a loading dose of 300 mg an anti-IL-36R antibody or antigen-binding fragment thereof at week 0 or 1, followed by maintenance doses comprising 150 mg of said anti-IL-36R antibody administered at 2-, 4-, 5-, 6-, 7-, 8-, 10-, or 12-week intervals. In a preferred embodiment, the invention relates to said loading and maintenance doses that are administered subcutaneously for the treatment of GPP in adults and adolescents from 12 years of age when not experiencing flare. In an exemplary embodiment, a subcutaneous loading dose of 300 mg (two 150 mg injections), are followed by 150 mg (one 150 mg injections) subcutaneously 12 weeks later and every 12 weeks thereafter.

In aspects of the invention related to any of the embodiments above, the loading dose and maintenance treatment dose(s) are administered parenterally, e.g., intravenously and/or subcutaneously.

In one embodiment related to any of the above aspects and embodiments, the anti-IL-36R antibody is administered in one or more subcutaneous dose(s), wherein the total loading dose of an anti-IL-36R antibody or antigen-binding fragment thereof is at least 300 mg-600 mg of said anti-IL-36R antibody, followed by maintenance doses comprising 150 mg-600 mg of said anti-IL-36R antibody administered subcutaneously to the subject at 4 week (q4w)-12-week (q12w) intervals. In one embodiment, a total loading dose of 300 mg of said anti-IL-36R antibody or antigen-binding fragment thereof is delivered subcutaneously at week 0 or 1, followed by maintenance doses comprising 150 mg of said anti-IL-36R antibody administered subcutaneously to the subject at 12-week (q12w) intervals. In another embodiment, a total loading dose of 600 mg of said anti-IL-36R antibody is delivered subcutaneously at week 0 or 1, followed by maintenance doses comprising 600 mg of said anti-IL-36R antibody administered subcutaneously to the subject at 12-week (q12w) intervals. In another embodiment, a total loading dose of 600 mg of said anti-IL-36R antibody is delivered subcutaneously at week 0 or 1, followed by maintenance doses comprising 300 mg of said anti-IL-36R antibody administered subcutaneously to the subject at 12-week (q12w) intervals. In another embodiment, a total loading dose of 600 mg of said anti-IL-36R antibody is delivered subcutaneously at week 0 or 1, followed maintenance doses comprising 600 mg of said anti-IL-36R antibody administered subcutaneously to the subject at 4-week (q4w) intervals. In a preferred embodiment, a total loading dose of 600 mg (e.g., four 150 mg injections) of said anti-IL-36R antibody is delivered subcutaneously at week 0 or 1, followed by maintenance doses comprising 300 mg (e.g., two 150 mg injections) of said anti-IL-36R antibody administered subcutaneously to the subject at 4 weeks after the last loading dose, and every 4-weeks (q4w) thereafter.

In one aspect, the invention relates to a method of the treatment of the occurrence of a GPP flare in a subject undergoing with maintenance treatment with the anti-IL36 antibody or antigen-binding fragment thereof according to any of the preceding embodiments, comprising administering to the subject at least one 900 mg intravenous (i.v.) dose of an anti-IL-36R antibody. In a related embodiment, treatment of a GPP flare is at any time during the maintenance treatment phase in a subject diagnosed as experience a flare, wherein GPP flare is defined as an increase in a GPP Physician Global Assessment (GPPGA) total score of ≥2, and a GPPGA pustulation subscore of ≥2. In a related embodiment, if the GPP flare symptoms persist, an additional 900 mg dose may be administered 1 week after the initial dose.

In one aspect, the invention relates to a method of improving the quality of life by at least 10% in a subject with a history of GPP symptoms, and/or with a confirmed history (diagnosis) of GPP based on the consensus diagnostic criteria defined by the ERASPEN Diagnostic Criteria (Website:eraspen.eu/home/rfp/diagnostic-criteria.html (access date: 9 May 2018); European Rare And Severe Psoriasis Expert Network (ERASPEN); 2018), comprising administering to the subject a loading dose of 600 mg of an anti-IL-36R antibody or antigen-binding fragment thereof at week 1; followed by maintenance doses comprising 300 mg of said anti-IL-36R antibody administered to the subject at 2-, 4-, 5-, 6-, 7-, 8-, 10-, or 12-week intervals. In a related embodiment, said method comprises administering to the subject a loading dose of 600 mg of said anti-IL-36R antibody at week 0 or 1, followed by maintenance doses comprising 600 mg of said anti-IL-36R antibody administered to the subject at 12-week (q12w) intervals. In a related embodiment, said method comprises administering to the subject a loading dose of 600 mg of said anti-IL-36R antibody at week 0 or 1, followed by maintenance doses comprising 300 mg of said anti-IL-36R antibody administered to the subject at 12-week (q12w) intervals. In a related embodiment, said method comprises administering to the subject a loading dose of 600 mg of said anti-IL-36R antibody at week 0 or 1, followed by maintenance doses comprising 600 mg of said anti-IL-36R antibody administered to the subject at 4-week (q4w) intervals. In a related embodiment, said method comprises administering to the subject a loading dose of 600 mg of said anti-IL-36R antibody at week 0 or 1, followed by maintenance doses comprising 300 mg of said anti-IL-36R antibody administered to the subject at 4-week (q4w) intervals.

In one aspect, the invention relates to a method of improving the quality of life by at least 10% in a subject with a history of GPP symptoms, and/or with a confirmed history (diagnosis) of GPP based on the consensus diagnostic criteria defined by the ERASPEN Diagnostic Criteria, said method comprising administering to a subject a loading dose of 300 mg of an anti-IL-36R antibody or antigen-binding fragment thereof at week 0 or 1, followed by maintenance doses comprising 150 mg of said anti-IL-36R antibody administered to the subject at 2-, 4-, 5-, 6-, 7-, 8-, 10-, or 12-week intervals. In a related embodiment, said method comprises administering to the subject a loading dose of 300 mg of said anti-IL-36R antibody at week 0 or 1, followed by maintenance doses comprising 150 mg of said anti-IL-36R antibody administered to the subject at 12-week (q12w) intervals.

In an embodiment relating to any of the above aspects, the subject with a history of GPP symptoms and/or with a confirmed history (diagnosis) of GPP based on the consensus diagnostic criteria defined by the ERASPEN Diagnostic Criteria, has a decrease in the occurrence of at least one GPP flare as measured by the proportion of patients with at least one flare event, wherein GPP flare is defined as an increase in a GPP Physician Global Assessment (GPPGA) total score of ≥2, and a GPPGA pustulation subscore of ≥2, from baseline up week 48 after the initiation of treatment.

In an embodiment relating to any of the above aspects, the subject with a history of GPP symptoms and/or with a confirmed history (diagnosis) of GPP based on the consensus diagnostic criteria defined by the ERASPEN Diagnostic Criteria, has a risk reduction in GPP flare occurrence, wherein GPP flare is defined as in increase in a GPP Physician Global Assessment (GPPGA) total score of ≥2 and a GPPGA pustulation subscore of ≥2, from baseline up to week 48 after the initiation of treatment.

In an embodiment relating to any of the above aspects, the subject with a history of GPP symptoms and/or with a confirmed history (diagnosis) of GPP based on the consensus diagnostic criteria defined by the ERASPEN Diagnostic Criteria, has a GPP Physician Global Assessment (GPPGA) total score of ≤1 and a GPPGA pustulation subscore of ≤1 before the administration of the first loading dose of an anti-IL-36R antibody.

In an embodiment relating to any of the above aspects, the subject with a history of GPP symptoms and/or with a confirmed history (diagnosis) of GPP based on the consensus diagnostic criteria defined by the ERASPEN Diagnostic Criteria, has a GPP Physician Global Assessment (GPPGA) total score of ≤1 and a GPPGA pustulation subscore of ≤1 after the administration of an initial loading dose of 300 mg or 600 mg of an anti-IL-36R antibody or antigen-binding fragment thereof administered subcutaneously at week 0 or 1, followed by at least one maintenance dose of 150, 300 mg, or 600 mg of said anti-IL-36R antibody administered to the subject at 2-, 4-, 5-, 6-, 7-, 8-, 10-, or 12-week intervals. In a related embodiment, the subject has a GPP Physician Global Assessment (GPPGA) total score of ≤1 and a GPPGA pustulation subscore of ≤1 after the administration of said loading and maintenance doses of said anti-IL-36R antibody up to at least 48 weeks following the initial loading dose.

In an embodiment relating to any of the above aspects, the subject with a history of GPP symptoms and/or with a confirmed history (diagnosis) of GPP based on the consensus diagnostic criteria defined by the ERASPEN Diagnostic Criteria, has a GPP Physician Global Assessment (GPPGA) total score of ≤1 and a GPPGA pustulation subscore of ≤1 before and after the administration of the first loading dose of an anti-IL-36R antibody or antigen-binding fragment thereof. In a related embodiment, the subject with a history of GPP flares has a GPP Physician Global Assessment (GPPGA) total score of ≤1 and a GPPGA pustulation subscore of ≤1 before and after the administration of the first loading dose of said anti-IL-36R antibody up to at least 48 weeks following the initial loading dose.

In one aspect, the invention relates to a method of treatment of generalized pustular psoriasis (GPP), including the treatment and prevention of flares of a subject with a history of GPP symptoms, and/or with a confirmed history (diagnosis) of GPP based on the consensus diagnostic criteria defined by the ERASPEN Diagnostic Criteria, and/or a GPPGA pustulation subscore of ≤1, said method comprising the steps of: (a) administering to the subject a loading dose of 600 mg of an anti-IL-36R antibody or antigen-binding fragment thereof at week 0 to 1, followed by maintenance doses comprising 600 mg of an anti-IL-36R antibody administered to the subject at 2-, 4-, 5-, 6-, 7-, 8-, 10- or 12-week intervals, wherein administration of the loading dose and/or the maintenance dose of said anti-IL-36R antibody is delivered subcutaneously, and; (b) assessing the GPP Physician Global Assessment (GPPGA) total and/or GPPGA pustulation subscore of the subject up to at least week 48 after the initial loading dose, wherein the time to the first GPP flare up to Week 48 after the initiation of treatment is defined by a GPPGA pustulation subscore of ≥2 and an increase in GPPGA total score by ≥2 from baseline. In another aspect, the invention relates to a method of treatment of generalized pustular psoriasis (GPP), including the treatment and prevention of flares of a subject with a history of GPP symptoms, and/or with a confirmed history (diagnosis) of GPP based on the consensus diagnostic criteria defined by the ERASPEN Diagnostic Criteria, and/or a GPPGA pustulation subscore of ≤1, said method comprising the steps of: (a) administering to the subject a loading dose of 600 mg of an anti-IL-36R antibody or antigen-binding fragment thereof at week 0 to 1, followed by maintenance doses comprising 300 mg of an anti-IL-36R antibody administered to the subject at 2-, 4-, 5-, 6-, 7-, 8-, 10- or 12-week intervals, wherein administration of the loading dose and/or the maintenance dose of said anti-IL-36R antibody is delivered subcutaneously, and; (b) assessing the GPP Physician Global Assessment (GPPGA) total and/or GPPGA pustulation subscore of the subject up to at least week 48 after the initial loading dose, wherein the time to the first GPP flare up to Week 48 after the initiation of treatment is defined by a GPPGA pustulation subscore of ≥2 and an increase in GPPGA total score by 2 from baseline. In a related embodiment, step a) of said method comprises administering to the subject a loading dose of 600 mg of an anti-IL-36R antibody or antigen-binding fragment thereof at week 0 to 1, followed by maintenance doses comprising 600 mg of said anti-IL-36R antibody administered to the subject at 12-week intervals, wherein administration of the loading dose and/or the maintenance dose of said anti-IL-36R antibody is delivered subcutaneously. In a related embodiment, step a) of said method comprises administering to the subject a loading dose of 600 mg of an anti-IL-36R antibody or antigen-binding fragment thereof at week 0 to 1, followed by maintenance doses comprising 300 mg of said anti-IL-36R antibody administered to the subject at 12-week intervals, wherein administration of the loading dose and/or the maintenance dose of said anti-IL-36R antibody is delivered subcutaneously. In a related embodiment, step a) of said method comprises administering to the subject a loading dose of 600 mg of said anti-IL-36R antibody at week 0 to 1, followed by maintenance doses comprising 600 mg of said anti-IL-36R antibody administered to the subject at 4-week intervals, wherein administration of the loading dose and/or the maintenance dose of said anti-IL-36R antibody is delivered subcutaneously. In a related embodiment, step a) of said method comprises administering to the subject a loading dose of 600 mg of said anti-IL-36R antibody at week 0 to 1, followed by maintenance doses comprising 300 mg of said anti-IL-36R antibody administered to the subject at 4-week intervals, wherein administration of the loading dose and/or the maintenance dose of said anti-IL-36R antibody is delivered subcutaneously.

In one aspect, the invention relates to a method treatment of generalized pustular psoriasis (GPP), including the treatment and prevention of flares of a subject with a history of GPP symptoms, and/or with a confirmed history (diagnosis) of GPP based on the consensus diagnostic criteria defined by the ERASPEN Diagnostic Criteria, and/or a GPPGA pustulation subscore of ≤1, said method comprising the steps of: (a) administering to the subject a loading dose of 300 mg of an anti-IL-36R antibody or antigen-binding fragment thereof at week 0 or 1, followed by a maintenance dose comprising at least one dose 150 mg of said anti-IL-36R antibody administered to the subject at 2-, 4-, 5-, 6-, 7-, 8-, 10-, or 12-week intervals, wherein administration of the loading dose and/or the maintenance dose of said anti-IL-36R antibody is delivered intravenously and/or subcutaneously; and (b) assessing the GPP Physician Global Assessment (GPPGA) total and/or the GPPGA pustulation subscore of the subject up to at least week 48 after the initial loading dose, wherein the time to the first GPP flare up to Week 48 is defined by a GPPGA pustulation subscore of ≥2 and an increase in GPPGA total score by 2 from baseline. In a related embodiment, step a) of said method comprises administering to the subject a loading dose of 300 mg of said anti-IL-36R antibody at week 0 to 1, followed by maintenance doses comprising 150 mg of an anti-IL-36R antibody administered to the subject at 12-week intervals, wherein administration of the loading dose and/or the maintenance dose of said anti-IL-36R antibody is delivered subcutaneously. In a related embodiment, the GPP Physician Global Assessment (GPPGA) total and/or the GPPGA pustulation subscore is measured after the initiation of treatment at Week 1, 4, 8, 12, 16, 20, 24, 28, 32, 36, 40, 44, 48, and/or 16 weeks after the last dose.

In an embodiment relating to any of the above aspects, the invention relates to a method of treatment of generalized pustular psoriasis (GPP), including the treatment and prevention of flares of a subject with a history of GPP symptoms and/or with a confirmed history (diagnosis) of GPP based on the consensus diagnostic criteria defined by the ERASPEN Diagnostic Criteria, wherein clinical improvement is defined as a reduced risk of GPP flare, and/or risk of worsening of Psoriasis Symptom Scale (PSS) up to Week 48 after the initiation of treatment, defined as a 4-point increase in total score from baseline. In a related embodiment, at least 10%, 20%, 30%, 40%, 50%, 60%, 70% or 80% of the GPP subjects treated with said anti-IL-36R antibody show a reduced risk of worsening of Psoriasis Symptom Scale (PSS) after the initiation of treatment at Week 1, 4, 8, 12, 16, 20, 24, 28, 32, 36, and/or 48 of the treatment.

In an embodiment relating to any of the above aspects, the invention relates to a method of treatment of generalized pustular psoriasis (GPP), including the treatment and prevention of flares in a subject with a history of GPP symptoms, and/or with a confirmed history (diagnosis) of GPP based on the consensus diagnostic criteria defined by the ERASPEN Diagnostic Criteria, wherein clinical improvement is defined by reduced risk of worsening of Dermatology Quality of Life Index (DLQI) up to Week 48 after the initiation of treatment, (worsening is defined as a 4-point increase in total score from baseline). In a related embodiment, at least 10%, 20%, 30%, 40%, 50%, 60%, 70% or 80% of the GPP subjects treated with said anti-IL-36R antibody show a reduced risk of worsening of Dermatology Quality of Life Index (DLQI) at Week 4, 8, 12, 24, 36, and/or 48 of the treatment.

In an embodiment relating to any of the above aspects or embodiments, the administration of an anti-IL-36R antibody or antigen-binding fragment thereof to a subject with a history of GPP symptoms, and/or with a confirmed history (diagnosis) of GPP based on the consensus diagnostic criteria defined by the ERASPEN Diagnostic Criteria, achieves one or more of the following results: (a) maintains a Generalized Pustular Psoriasis Global Assessment (GPPGA) pustulation subscore of 0 after administering said anti-IL-36R antibody, at all visits up to Week 48, without treatment for GPP flare, or investigator-prescribed Standard of Care (SoC); and/or
(b) maintains a GPPGA total score of 0 or 1 after administration of said anti-IL-36R antibody up to week 48 after the initial loading dose; or
(c) experiences sustained remission, defined as a subject treated with said anti-IL-36R antibody who maintains a GPPGA score of 0 or 1 (clear or almost clear) at all visits up to Week 48, without treatment for GPP flare, or investigator-prescribed Standard of Care (SoC).

An embodiment of the invention, related to any of aspects above relates the proportion of subjects with a history of GPP symptoms and/or with a confirmed history (diagnosis) of GPP based on the consensus diagnostic criteria defined by the ERASPEN Diagnostic Criteria, who achieve any of the above results due to the administration of an anti-IL-36R antibody that is greater than subjects on placebo treatment for any of the end points recited.

In another aspect, the present invention relates to a method of treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria, including (a) obtaining a biological sample from said subject, wherein the biological sample is obtained from source including lesional skin or whole blood; (b) determining the gene express profile of one or more of genes; (c) administering to the subject an effective amount of the anti-IL-36R antibody according to any embodiments relating to any of the aspects above. In a related embodiment, the one or more of genes that are profiled are IL12B, IL1B, IL6, CXCL1, IL23A, TNF, IL17C, IL24 or IL1B in lesional skin, and IL1B, S100A9, S100A12, S100A8, MMP25, MMP9 or CD177 in whole blood.

In another aspect, the present invention relates to a method for detecting the presence or absence of a beneficial response in a GPP patient after administration of an anti-interleukin-36 receptor antibody (anti-IL-36R antibody), comprising: a) obtaining a biological sample from the patient pre-treatment and post-treatment after administration of the anti-IL-36R antibody; b) measuring in each sample the level of one or more biomarkers, or the level of expression of one or more biomarkers in each sample pre-treatment and post-treatment; c) comparing the pre-treatment level to post-treatment level of the biomarkers; and d) determining the difference in levels between the pre-treatment sample and the post-treatment sample reflects a beneficial response in the patient, wherein the one or more biomarkers comprise microRNAs selected from the group consisting of miR-223-3p, miR-223-5p, miR-1304-3p, miR-485-5p, or miR-337-5p, wherein downregulation or upregulation post-treatment as compared to pre-treatment baseline, indicates beneficial response. In an exemplary embodiment, the biological sample is a skin biopsy, blood, plasma, or serum sample. In a preferred embodiment, the one or more of microRNAs are miR-223-5p or miR-223-3 in lesional skin or serum.

In another aspect, the present invention relates to a method for detecting the presence or absence of a beneficial response in a GPP patient after administration of an anti-interleukin-36 receptor antibody (anti-IL-36R antibody), comprising: a) providing a biological sample from the patient pre-treatment and post-treatment after administration of the anti-IL-36R antibody; b) measuring ex vivo in each sample the level of one or more biomarkers, or the level of expression of one or more biomarkers in each sample pre-treatment and post-treatment; c) comparing the pre-treatment level to post-treatment level of the biomarkers; and d) determining the difference in levels between the pre-treatment sample and the post-treatment sample reflects a beneficial response in the patient, wherein the one or more biomarkers comprise microRNAs selected from the group consisting of miR-223-3p, miR-223-5p, miR-1304-3p, miR-485-5p, or miR-337-5p, wherein downregulation or upregulation post-treatment as compared to pre-treatment baseline, indicates beneficial response. In an exemplary embodiment, the biological sample is a skin biopsy, blood, plasma, or serum sample. In a preferred embodiment, the one or more of microRNAs are miR-223-5p or miR-223-3 in lesional skin or serum.

In another aspect, the present invention relates use of an anti-interleukin-36 receptor antibody (anti-IL-36R antibody) in a method for detecting the presence or absence of a beneficial response in a GPP patient after administration of said anti-interleukin-36 receptor antibody, comprising: a) providing a biological sample from the patient pre-treatment and post-treatment after administration of the anti-IL-36R antibody; b) measuring in each sample (e.g. ex vivo) the level of one or more biomarkers, or the level of expression of one or more biomarkers in each sample pre-treatment and post-treatment; c) comparing the pre-treatment level to post-treatment level of the biomarkers; and d) determining the difference in levels between the pre-treatment sample and the post-treatment sample reflects a beneficial response in the patient, wherein the one or more biomarkers comprise microRNAs selected from the group consisting of miR-223-3p, miR-223-5p, miR-1304-3p, miR-485-5p, or miR-337-5p, wherein downregulation or upregulation post-treatment as compared to pre-treatment baseline, indicates beneficial response. In an exemplary embodiment, the biological sample is a skin biopsy, blood, plasma, or serum sample. In a preferred embodiment, the one or more of microRNAs are miR-223-5p or miR-223-3 in lesional skin or serum.

In another aspect, the present invention relates to a method for detecting the presence or absence of a beneficial response in a GPP patient after administration of an anti-interleukin-36 receptor antibody (anti-IL-36R antibody), wherein the levels of one or more of microRNAs miR-223-5p and miR-223-3 in lesional skin or serum is correlated with GPPASI and/or GPPGA scores wherein treatment or prevention of flares of a subject with a history of GPP symptoms is measured as achieving one or more of the following results:
(a) maintenance of a Generalized Pustular Psoriasis Global Assessment (GPPGA) pustulation subscore of 0 or 1 and maintenance or decrease in the level of miR-223-5p and/or miR-223-3 after administering said anti-IL-36R antibody up week 48 after the initial loading dose;
(b) maintenance of a GPPGA total score of 0 or 1 and maintenance or decrease in the level of miR-223-5p and/or miR-223-3 after administration of said anti-IL-36R antibody up to week 48 after the initial loading dose;
(c) a sustained remission of GPP symptoms, defined as a subject treated with said anti-IL-36R antibody who maintains a GPPGA score of 0 or 1 (clear or almost clear) and maintenance or decrease in the level of miR-223-5p and/or miR-223-3 at all visits up to Week 48, without intake of GPP flare medication, or investigator-prescribed Standard of Care (SoC);
(d) a decrease in the occurrence of GPP flare(s) and maintenance or decrease in the level of miR-223-5p and/or miR-223-3, wherein GPP flare is defined as in increase in GPP Physician Global Assessment (GPPGA) total score of ≥2 and a GPPGA pustulation subscore of ≥2, from baseline up to week 48; or
(e) a prolongation of the time to a first GPP flare as measured by the time from baseline to the onset of said first GPP flare, wherein GPP flare is defined as in increase in the GPP Physician Global Assessment (GPPGA) total score of ≥2, and a GPPGA pustulation subscore of ≥2, and an increase in the level of miR-223-5p and/or miR-223-3 from baseline up to week 48.

In another aspect, the present invention relates to a method for detecting the presence or absence of a beneficial response in a GPP patient after administration of an anti-interleukin-36 receptor antibody (anti-IL-36R antibody), wherein the levels of one or more of microRNAs miR-223-5p and miR-223-3 in lesional skin or serum is correlated with GPPASI and/or GPPGA scores, wherein the levels of the one or more of micro RNAs is determined or has been determined ex vivo in a sample, wherein treatment or prevention of flares of a subject with a history of GPP symptoms is measured as achieving one or more of the following results:
(a) maintenance of a Generalized Pustular Psoriasis Global Assessment (GPPGA) pustulation subscore of 0 or 1 and maintenance or decrease in the level of miR-223-5p and/or miR-223-3 after administering said anti-IL-36R antibody up week 48 after the initial loading dose;
(b) maintenance of a GPPGA total score of 0 or 1 and maintenance or decrease in the level of miR-223-5p and/or miR-223-3 after administration of said anti-IL-36R antibody up to week 48 after the initial loading dose;
(c) a sustained remission of GPP symptoms, defined as a subject treated with said anti-IL-36R antibody who maintains a GPPGA score of 0 or 1 (clear or almost clear) and maintenance or decrease in the level of miR-223-5p and/or miR-223-3 at all visits up to Week 48, without intake of GPP flare medication, or investigator-prescribed Standard of Care (SoC);
(d) a decrease in the occurrence of GPP flare(s) and maintenance or decrease in the level of miR-223-5p and/or miR-223-3, wherein GPP flare is defined as in increase in GPP Physician Global Assessment (GPPGA) total score of ≥2 and a GPPGA pustulation subscore of ≥2, from baseline up to week 48; or
(e) a prolongation of the time to a first GPP flare as measured by the time from baseline to the onset of said first GPP flare, wherein GPP flare is defined as in increase in the GPP Physician Global Assessment (GPPGA) total score of ≥2, and a GPPGA pustulation subscore of ≥2, and an increase in the level of miR-223-5p and/or miR-223-3 from baseline up to week 48.

In another aspect, the present invention relates to use of an anti-interleukin-36 receptor antibody (anti-IL-36R antibody) in a method for detecting the presence or absence of a beneficial response in a GPP patient after administration of said anti-interleukin-36 receptor antibody, wherein the levels of one or more of microRNAs miR-223-5p and miR-223-3 in lesional skin or serum is correlated with GPPASI and/or GPPGA scores, wherein treatment or prevention of flares of a subject with a history of GPP symptoms is measured as achieving one or more of the following results:
(a) maintenance of a Generalized Pustular Psoriasis Global Assessment (GPPGA) pustulation subscore of 0 or 1 and maintenance or decrease in the level of miR-223-5p and/or miR-223-3 after administering said anti-IL-36R antibody up week 48 after the initial loading dose;
(b) maintenance of a GPPGA total score of 0 or 1 and maintenance or decrease in the level of miR-223-5p and/or miR-223-3 after administration of said anti-IL-36R antibody up to week 48 after the initial loading dose;
(c) a sustained remission of GPP symptoms, defined as a subject treated with said anti-IL-36R antibody who maintains a GPPGA score of 0 or 1 (clear or almost clear) and maintenance or decrease in the level of miR-223-5p and/or miR-223-3 at all visits up to Week 48, without intake of GPP flare medication, or investigator-prescribed Standard of Care (SoC);
(d) a decrease in the occurrence of GPP flare(s) and maintenance or decrease in the level of miR-223-5p and/or miR-223-3, wherein GPP flare is defined as in increase in GPP Physician Global Assessment (GPPGA) total score of ≥2 and a GPPGA pustulation subscore of ≥2, from baseline up to week 48; or
(e) a prolongation of the time to a first GPP flare as measured by the time from baseline to the onset of said first GPP flare, wherein GPP flare is defined as in increase in the GPP Physician Global Assessment (GPPGA) total score of ≥2, and a GPPGA pustulation subscore of ≥2, and an increase in the level of miR-223-5p and/or miR-223-3 from baseline up to week 48.

In an embodiment related to any of the above, the present invention relates to a method wherein the levels of biomarkers are determined by small RNA sequencing, qPCR, or ELISA and IHC.

In another aspect, the present invention relates to a method for detecting the presence or absence of a beneficial response in a GPP patient after administration of an anti-IL-36R antibody further comprising continuing the administration of doses of the anti-IL-36R antibody to the patient if the difference in levels between the pre-treatment sample and the post-treatment reflects a beneficial response in the patient.

In another aspect, the present invention relates to use of an anti-interleukin-36 receptor antibody (anti-IL-36R antibody) in a method for detecting the presence or absence of a beneficial response in a GPP patient after administration of said anti-IL-36R antibody further comprising continuing the administration of doses of the anti-IL-36R antibody to the patient if the difference in levels between the pre-treatment sample and the post-treatment reflects a beneficial response in the patient.

In another aspect, the present invention relates to a method for determining whether a potential therapeutic agent is efficacious in the treatment and prevention of GPP comprising:
(a) obtaining a first biological sample from a GPP patient prior to flare and/or prior to being treated with the potential therapeutic agent;
(b) treating the GPP patient with the potential therapeutic agent;
(c) obtaining a second biological sample from the GPP patient after being treated with the potential therapeutic agent;
(d) measuring in said first and second sample the levels of expression of one or more biomarkers; and
(e) comparing the biomarker levels in the second sample to the levels in the first sample,
wherein changes (e.g., lower or higher) in biomarker levels in the second sample than in the first sample indicate that the potential therapeutic agent is efficacious, and further wherein the one or more biomarkers comprise microRNAs selected from the group consisting of miR-223-3p, miR-223-5p, miR-1304-3p, miR-485-5p, or miR-337-5p.

In another aspect, the present invention relates to a method for determining whether a potential therapeutic agent is efficacious in the treatment and prevention of GPP comprising:
(a) providing a first biological sample from a GPP patient prior to flare and/or prior to being treated with the potential therapeutic agent;
(b) treating the GPP patient with the potential therapeutic agent;
(c) providing a second biological sample from the GPP patient after being treated with the potential therapeutic agent;
(d) measuring ex vivo in said first and second sample the levels of expression of one or more biomarkers; and
(e) comparing the biomarker levels in the second sample to the levels in the first sample,
wherein changes (e.g., lower or higher) in biomarker levels in the second sample than in the first sample indicate that the potential therapeutic agent is efficacious, and further wherein the one or more biomarkers comprise microRNAs selected from the group consisting of miR-223-3p, miR-223-5p, miR-1304-3p, miR-485-5p, or miR-337-5p.

In another aspect, the present invention relates to use of an anti-interleukin-36 receptor antibody (anti-IL-36R antibody) in a method for determining whether a potential therapeutic agent is efficacious in the treatment and prevention of GPP comprising:
(a) providing a first biological sample from a GPP patient prior to flare and/or prior to being treated with the potential therapeutic agent;
(b) treating the GPP patient with the potential therapeutic agent;
(c) providing a second biological sample from the GPP patient after being treated with the potential therapeutic agent;
(d) measuring (e.g. ex vivo) in said first and second sample the levels of expression of one or more biomarkers; and
(e) comparing the biomarker levels in the second sample to the levels in the first sample,
wherein changes (e.g., lower or higher) in biomarker levels in the second sample than in the first sample indicate that the potential therapeutic agent is efficacious, and further wherein the one or more biomarkers comprise microRNAs selected from the group consisting of miR-223-3p, miR-223-5p, miR-1304-3p, miR-485-5p, or miR-337-5p.

In a related aspect, the present invention relates to a method wherein changes (e.g., lower or higher) in biomarker levels in the second sample when compared to the first sample are correlated with improvement in clinical efficacy measures. A further embodiment relates to continuing treatment of the patient if said biomarker levels in the second sample change (e.g., are higher or lower) as compared to the first sample.

In a related aspect, the present invention relates to a method of treatment of generalized pustular psoriasis (GPP) in a subject with a history of GPP when not experiencing a flare comprising: a) determining whether to initiate treatment of the subject, modify the treatment dose, modify the dosing interval, or discontinue treatment, based any of the preceding embodiments; and b) modifying the treatment regimen based on the determination.

In a related aspect, the present invention relates to use of an anti-interleukin-36 receptor antibody (anti-IL-36R antibody) in a method of treatment of generalized pustular psoriasis (GPP) in a subject with a history of GPP when not experiencing a flare comprising: a) determining whether to initiate treatment of the subject, modify the treatment dose, modify the dosing interval, or discontinue treatment, based any of the preceding embodiments; and b) modifying the treatment regimen based on the determination.

In another aspect, the present invention relates to a method of monitoring patient response to a GPP treatment comprising:
(a) obtaining a first biological sample from the patient;
(b) measuring the level of one or more biomarkers in said first biological sample, wherein said one or more biomarkers comprise microRNAs selected from the group consisting of miR-223-3p, miR-223-5p, miR-1304-3p, miR-485-5p, or miR-337-5p;
(c) administering a treatment compound to the patient;
(d) obtaining a second biological sample from the patient;
(e) measuring the level of said one or more biomarkers in said second biological sample; and
(f) comparing the levels of the one or more biomarkers obtained from first and second biological samples;
wherein upregulation or downregulation post-treatment as compared to pre-treatment baseline indicates an effective response.

In another aspect, the present invention relates to a method of monitoring patient response to a GPP treatment comprising:
(g) obtaining a first biological sample from the patient;
(h) measuring the level of one or more biomarkers in said first biological sample, wherein said one or more biomarkers comprise microRNAs selected from the group consisting of miR-223-3p, miR-223-5p, miR-1304-3p, miR-485-5p, or miR-337-5p;
(i) administering a treatment compound to the patient;
(j) obtaining a second biological sample from the patient;
(k) measuring the level of said one or more biomarkers in said second biological sample; and
(l) comparing the levels of the one or more biomarkers obtained from first and second biological samples;
wherein upregulation or downregulation post-treatment as compared to pre-treatment baseline indicates an effective response.

In another aspect, the present invention relates to use of an anti-interleukin-36 receptor antibody (anti-IL-36R antibody) in a method of monitoring patient response to a GPP treatment comprising:
(m) providing a first biological sample from the patient;
(n) measuring the level of one or more biomarkers in said first biological sample (e.g. ex vivo), wherein said one or more biomarkers comprise microRNAs selected from the group consisting of miR-223-3p, miR-223-5p, miR-1304-3p, miR-485-5p, or miR-337-5p;
(o) administering a treatment compound to the patient;
(p) providing a second biological sample from the patient;
(q) measuring the level of said one or more biomarkers in said second biological sample (e.g. ex vivo); and
(r) comparing the levels of the one or more biomarkers provided from first and second biological samples;
wherein upregulation or downregulation post-treatment as compared to pre-treatment baseline indicates an effective response.

In another aspect, the present invention relates to a method for monitoring patient compliance with a drug treatment protocol for the treatment and prevention of GPP flares comprising:
(a) obtaining a first biological sample from the patient;
(b) measuring the level of one or more biomarkers in said first biological sample, wherein said one or more biomarkers comprise microRNAs selected from the group consisting of miR-223-3p, miR-223-5p, miR-1304-3p, miR-485-5p, or miR-337-5p;
(c) administering a treatment compound to the patient;
(d) obtaining a second biological sample from the patient;
(e) measuring the level of said one or more biomarkers in said second biological sample; and
(f) comparing the levels of the one or more biomarkers obtained from first and second biological samples;
wherein upregulation or downregulation post-treatment as compared to pre-treatment baseline indicates patient compliance with said drug treatment protocol.

In another aspect, the present invention relates to a method for monitoring patient compliance with a drug treatment protocol for the treatment and prevention of GPP flares comprising:
(g) providing a first biological sample from the patient;
(h) measuring the level of one or more biomarkers in said first biological sample ex vivo, wherein said one or more biomarkers comprise microRNAs selected from the group consisting of miR-223-3p, miR-223-5p, miR-1304-3p, miR-485-5p, or miR-337-5p;
(i) administering a treatment compound to the patient;
(j) providing a second biological sample from the patient;
(k) measuring the level of said one or more biomarkers in said second biological sample ex vivo; and
(l) comparing the levels of the one or more biomarkers obtained from first and second biological samples;
wherein upregulation or downregulation post-treatment as compared to pre-treatment baseline indicates patient compliance with said drug treatment protocol.

In another aspect, the present invention relates to use of an anti-interleukin-36 receptor antibody (anti-IL-36R antibody) in a method for monitoring patient compliance with a drug treatment protocol for the treatment and prevention of GPP flares comprising:
(m) providing a first biological sample from the patient;
(n) measuring the level of one or more biomarkers in said first biological sample (e.g. ex vivo), wherein said one or more biomarkers comprise microRNAs selected from the group consisting of miR-223-3p, miR-223-5p, miR-1304-3p, miR-485-5p, or miR-337-5p;
(o) administering a treatment compound to the patient;
(p) providing a second biological sample from the patient;
(q) measuring the level of said one or more biomarkers in said second biological sample (e.g. ex vivo); and
(r) comparing the levels of the one or more biomarkers obtained from first and second biological samples;
wherein upregulation or downregulation post-treatment as compared to pre-treatment baseline indicates patient compliance with said drug treatment protocol.

In another aspect of the above, the present invention relates to a method of monitoring patient response to a GPP treatment wherein the level of the one or more biomarkers in the second biological sample is decreased by at least about 20%, 30%, 40%, 50%, 55%, 60%, 65%, 65%, 70%, 75%, 80%, 85%, 90%, or 95% or more as compared to the level in the first biological sample.

In an exemplary embodiment related to the above, the present invention relates to method of any of the above wherein the biological sample is a skin biopsy, blood, plasma, or serum sample.

In yet another embodiment related to the above, the invention relates to a method wherein the treatment compound is an anti-IL-36R antibody.

In a related embodiment, the present invention relates to a method of any of the above wherein the levels of biomarkers are determined by small RNA sequencing or ELISA and IHC.

It will be understood that any of the herein disclosed methods, administration schemes and/or dosing regimens also equally apply to the use of any of the disclosed anti-IL-36R antibodies in such methods, administration schemes and/or dosing regimens, and any combinations of the aforementioned: e.g., an anti-IL-36R antibody, as disclosed herein, for use in the treatment, prevention, reducing and/or amelioration of any of the disclosed diseases and/or conditions. In other words, the invention also provides for the use of an anti-IL-36R antibody, as disclosed herein, for the manufacture of a medicament for the treatment, prevention, reducing and/or amelioration of any of the disclosed diseases and/or conditions. Alternatively, an anti-IL-36R antibody, as disclosed herein, for use in the treatment, prevention, reducing and/or amelioration of any of the disclosed diseases and/or conditions, said use further comprising any of the herein disclosed methods, administration schemes and/or dosing regimens.

Additional features and advantages of the present invention will be set forth in the description below, and in part will be apparent from the description, or may be learned by practice of the subject technology. It is to be understood that both the foregoing general description and the following detailed description are exemplary and explanatory and are intended to provide further explanation of the present invention as claimed.

### BRIEF DESCRIPTION OF THE DRAWINGS

The accompanying drawings, which are included to provide further understanding of the present invention and are incorporated in and constitute a part of this specification, illustrate aspects of the subject technology and together with the description explain the principles of the present invention.

FIG. 1 Phase IIb Clinical Study Design: proof-of-clinical-concept study aimed to explore the effect of spesolimab in subjects with a history of GPP symptoms for the prevention of acute GPP flares. Overall study design, with loading and maintenance treatment arms, and handling of GPP flares during the randomized maintenance treatment period. *GPP flare defined as an increase in GPPGA total score of ≥2 from baseline and the pustular component of GPPGA of ≥2.

FIG. 2A-C Time to the first GPP flare up to Week 48. (A) Table showing results of the formal testing of the primary and key secondary endpoints. (B) Kaplan-Meier plot showing the estimated probability of a first GPP flare over 48 weeks for all treatment groups. The use of medication with IV OL spesolimab or other investigator-prescribed medication was considered to be a GPP flare. (C) Bar chart showing the proportion of patients with ≥1 GPP flare up to Week 48. A multiple imputation method for binary endpoints with monotone missing assessments using sequential logistic regression method was utilized. The stratified Cochran-Mantel-Haenszel test was performed for each dose level of spesolimab vs. placebo, stratified by use of systemic GPP medication at randomization.

FIG. 3 Subgroup analyses for the time to the first GPP flare up to Week 48 for spesolimab high dose vs placebo.

FIG. 4A-C (A) Table showing results of the formal testing of other secondary endpoints: worsening of PSS and worsening of DLQI up to Week 48. For both scores, worsening was defined as a 4-point increase in total score from baseline. (B, C) Kaplan-Meier plot showing the estimated probability of a first worsening of PSS and DLQI scores, respectively. The use of OL spesolimab medication or other investigator-prescribed medication was considered to be an event. †Nominal P-value; statistical significance was not achieved in previous families in the statistical testing hierarchy.CI, confidence interval; DLQI, Dermatology Life Quality Index; GPP, generalized pustular psoriasis; IV, intravenous; n.c., not calculable; OL, open-label; P10, estimated probability of first GPP flare=0.1; P25, estimated probability of first GPP flare=0.25; PSS, Psoriasis Symptom Scale; SC, subcutaneous.

FIG. 5A-E (A) A graphic representation of the model-predicted concentration-time profiles for 300 mg delivered IV versus 300 mg delivered s.c over a period of 12 weeks. (B) A graphic representation of the model-predicted concentration-time profiles of 900 mg IV vs 600 mg SC spesolimab. (C) A graphic representation of the model-predicted concentration-time profiles of 900 mg IV×2 vs 600 mg SC×2 spesolimab. (D) A graphic representation of the model-predicted concentration-time profiles of 900 mg IV vs 2250 mg SC spesolimab. (E) A summary of exposure metrics after single IV or SC dose in patients with GPP.

FIG. 6A-B (A) Histopathological analysis of select neutrophilic proteins in representative patients; Patient A and Patient B. Representative patient A is IL36RN mutation positive, presenting with pustules, scaling, and had been treated with cyclosporine before enrollment in the spesolimab trial. Patient A was randomized to receive high dose spesolimab and did not flare throughout the study. In comparison, representative patient B did not have IL36RN mutations, had high neutrophil counts, and were treated with acitretin before enrollment. Patient B was randomized to receive placebo and went on to receive rescue spesolimab treatment for a flare during the study. Shows both at a baseline visit and at VOL, open label visit (patient A) or VR1, rescue visit 1; VR6, rescue visit 6 (patient B), both showing reduced expression of neutrophilic (neutrophil elastase, lipocalin [LCN]), IL-36R and inflammatory markers (S100A7, human β defensin 2 [hBD2]), indicating reduced inflammation and neutrophilic pustule formation. (B) A schematic of the treatment scenarios of patients in the Effisayil 2 trial, and the clinical morphology of patient A at baseline and at week 48, and rescue spesolimab treatment in patient B demonstrating resolved GPP flare skin symptoms within 4 weeks.

FIG. 7A-B miRNA expression changes in GPP lesions compared to healthy controls (HC). (A) PCA plot including confidence ellipses per group (HC, non-lesional (nL) and lesional (L) skin). (B) Volcano plot representing increased expression of 173 miRNAs and decreased expression of 160 miRNAs in skin of GPP patients compared to healthy controls. The horizontal line represents an adj. p-value of 0.05. The vertical lines represent a FC of +1.5 and −1.5. Differentially expressed miRNAs are shown in the upper left and right quadrants in boxes and top deregulated miRNAs are indicated.

FIG. 8A-B miRNA expression changes in GPP lesions after treatment with spesolimab. (A) PCA plot including confidence ellipses per group (healthy controls (HC), lesional (L) skin pre- and post-spesolimab treatment and non-lesional (nL) skin pre-spesolimab. (B) The volcano plot illustrates the deregulated miRNAs in lesional skin post-spesolimab (n=12) compared to baseline (n=9). The horizontal line represents an p-value of 0.05. The vertical lines represent a fold FC of +1.5 and −1.5. Differentially expressed miRNAs following spesolimab treatment are shown in the upper left and right quadrants in boxes.

FIG. 9A-B miRNA expression changes in serum obtained from GPP patients compared to healthy controls (HC). (A) PCA plot including confidence ellipses per group (healthy controls (HC), GPP patients pre-spesolimab). (B) Volcano plot representing increased expression of 69 miRNAs and decreased expression of 45 miRNAs in serum of GPP patients compared to healthy controls. The horizontal line represents an adj. p-value of 0.05. The vertical lines represent a FC of +1.5 and −1.5. Differentially regulated miRNAs are in the upper left and right quadrants in boxes where represented miRNAs with the strongest change in expression are indicated.

FIG. 10A-B Transcriptomic changes in GPP serum after treatment with spesolimab. (A) Venn diagram. Confidence ellipses are shown per group. (B) The volcano plot illustrates the deregulated miRNAs in lesional GPP post-spesolimab (n=11) compared to baseline (n=12). The horizontal line represents an p-value of 0.05. The vertical lines represent a fold FC of +1.5 and −1.5. Differentially expressed miRNAs following spesolimab treatment are shown in red.

FIG. 11A-B Overlap of differentially expressed miRNAs in lesion skin and serum from patients with GPP compared to healthy controls and treated with spesolimab. Number of differentially expressed miRNAs in lesional GPP (n=9) and serum (n=12) of patients with GPP compared to healthy controls (skin n=10, serum n=20). (A) Venn-diagram illustrates the overlap of differentially expressed miRNAs in lesional GPP and serum from patients with GPP compared to healthy controls and after spesolimab treatment. (B) Fold changes and corresponding P values of pairwise comparisons of the selected 5 miRNAs.

FIG. 12 A-B Expression levels of selected miRNAs were determined by RT-qPCR in skin: (A) Confirmation of miRNA expression in skin in GPP patients at baseline (n=9) compared to healthy controls (HC; n=10). (B) Deregulation in skin post-spesolimab treatment (n=12) compared to baseline (pre-spesolimab). Each sample is represented by a black dot and box plots indicate the median POC (percent of control). Statistical analysis was performed by a nonparametric Mann-Whitney test; ****P≤0.0001, ***P≤0.001, **P≤0.01, *P≤0.05.

FIG. 13 A-B Expression levels of selected miRNAs were determined by RT-qPCR in serum: (A) Confirmation of miRNA expression in serum in GPP patients at baseline (n=12) compared to healthy controls (HC; n=20). (B) Deregulation in serum post-spesolimab treatment (n=11) compared to baseline (pre-spesolimab) (D). Each sample is represented by a black dot and box plots indicate the median POC (percent of control). Statistical analysis was performed by a nonparametric Mann-Whitney test; ****P≤0.0001, ***P≤0.001, **P≤0.01, *P≤0.05.

FIG. 14A-C Correlation of differentially expressed miRNAs in skin and serum with clinical parameters measured by GPPASI and GPPGA scores. (A) Correlation of expression levels of miR-223-3p and miR-233-5p [log 2 CPM] taken from skin with GPPASI score. (B) Correlation of expression levels of miR-223-3p and miR-233-5p [log 2 CPM] taken from skin with GPPGA score. (C) Correlation of expression levels of miR-223-3p and miR-233-5p [log 2 CPM] taken from serum with GPPGA score. Black circles represent lesional GPP at baseline whereas black dots indicate lesional GPP post-spesolimab treatment. The respective Spearman correlation coefficient, Rs, is shown within the plots. ****P<0.0001, **P≤0.01, *P<0.05.

### DETAILED DESCRIPTION OF THE INVENTION

In the following detailed description, numerous specific details are set forth to provide a full understanding of the present invention. It will be apparent, however, to one ordinarily skilled in the art that the subject technology may be practiced without some of these specific details. In other instances, well-known structures and techniques have not been shown in detail so as not to obscure the present invention.

Unless defined otherwise, all technical and scientific terms used herein have the same meaning as commonly understood by one of ordinary skill in the art to which this invention belongs.

The inventors have discovered inter alia that the signs and symptoms of GPP can be prevented and/or significantly alleviated symptoms by increasing the time between flares and/or decreasing the duration or severity of flares in subjects with a history of GPP, and/or with a confirmed history (diagnosis) of GPP based on the consensus diagnostic criteria defined by the ERASPEN Diagnostic Criteria by inhibiting the interleukin-36 pathway with a humanized anti-interleukin-36R (anti-IL-36R) monoclonal antibody of the present invention. Moreover, maintenance treatment with a humanized anti-interleukin-36R (anti-IL-36R) monoclonal antibody of the present invention resulted in the sustained remission in GPP subjects who demonstrated no clinical symptoms of acute generalized pustular psoriasis and no recurrence of GPP flares up to 48 weeks after the first loading dose administration.

The invention therefore relates to compositions and methods for the treatment of generalized pustular psoriasis (GPP), including the treatment and prevention of flares in a subject. More specifically, the invention relates to compositions and methods for treating and/or prophylaxis of GPP, acute GPP, chronic GPP, and/or GPP flares in a mammal with an anti-IL36R antibody or an antigen-binding fragment thereof of the present invention. The compositions and methods include administering to the mammal a therapeutically effective amount of an anti-IL-36R antibody or an antigen-binding fragment thereof prior to the occurrence of a flare in a patient with history of GPP, wherein the anti-IL-36R antibody is administered as a dosing regimen comprising a loading dose of 300 mg-600 mg delivered subcutaneously at week 0 or 1, followed by at least one maintenance dose, more preferably a series of maintenance doses of 150 mg-600 mg delivered subcutaneously at 2-, 4-, 5-, 6, 7-, 8-, 9-, 10-, 11-, or 12-week intervals which prevent the occurrence GPP flares of or frequency of the GPP symptoms. In exemplary embodiments of the invention the anti-IL-36R antibody is administered as a loading dose of 600 mg, followed by maintenance doses of 300 mg delivered at 4-week intervals; or a loading dose of 600 mg, followed by maintenance doses of 300 mg delivered at 12-week intervals; or a loading dose of 300 mg, followed by maintenance doses of 150 mg delivered at 12-week intervals.

TABLE 1
Treatment arms in Effisayil 2 Clinical Trial

| anti-IL36R | | |
|---|---|---|
| antibody | Loading dose | Subsequent doses |
| Spesolimab | 600 mg subcutaneously | 300 mg subcutaneously every |
| | | 4 weeks |
| Spesolimab | 600 mg subcutaneously | 300 mg subcutaneously every |
| | | 12 weeks |
| Spesolimab | 300 mg subcutaneously | 150 mg subcutaneously every |
| | | 12 weeks |
| Spesolimab | subcutaneous treatment | subcutaneous treatment every |
| | | 4 weeks |

Without wishing to be bound by theory it is believed that anti-IL-36R antibodies or antigen-binding fragments thereof bind to human anti-IL-36R and thus interfere with the binding of IL-36 agonists, and in doing so block at least partially the signaling cascade from the IL-36R to inflammatory mediators. The anti-IL36R antibodies of the present invention are disclosed in U.S. Pat. No. 9,023,995 or WO2013/074569, the entire content of each of which is incorporated herein by reference.

### I. DEFINITIONS

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

A phrase such as “an aspect” does not imply that such aspect is essential to the present invention or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure.

### II. ANTIBODIES OF THE PRESENT INVENTION

The anti-IL36R antibodies of the present invention are disclosed in U.S. Pat. No. 9,023,995 or WO2013/074569, the entire content of each of which is incorporated herein by reference.

The terms, “antibody”, “anti-IL-36R antibody”, “humanized anti-IL-36R antibody”, “humanized anti-IL-36R epitope antibody”, and “variant humanized anti-IL-36R epitope antibody” specifically encompass monoclonal antibodies (including full length monoclonal antibodies), polyclonal antibodies, multispecific antibodies (e.g., bispecific antibodies), antibodies with minor modifications such as N- and/or C-terminal truncation, and antibody fragments such as variable domains and other portions of antibodies that exhibit a desired biological activity, e.g., IL-36R binding.

The term “monoclonal antibody” (mAb) refers to an antibody that is highly specific, being directed against a single antigenic determinant, an “epitope”. Therefore, the modifier “monoclonal” is indicative of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352: 624-628, and Marks et al., 1991, J. Mol. Biol. 222: 581-597.

The term “monomer” refers to a homogenous form of an antibody. For example, for a full-length antibody, monomer means a monomeric antibody having two identical heavy chains and two identical light chains.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81: 6851-6855).

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

According to certain embodiments, the antibodies used in the methods of the present invention specifically bind IL-36R. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-36R, as used in the context of the present invention, includes antibodies that bind IL-36R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human IL-36R may, however, have cross-reactivity to other antigens, such as IL-36R molecules from other (non-human) species.

In certain exemplary embodiments related to any aspects of the present invention, the anti-IL-36R antibody or antigen-binding fragment thereof that can be used in the context of the methods of the present invention includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
V. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3) or
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
VII. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
(i) a light chain variable region comprising the amino acid sequence of SEQ ID NO: 77; and a heavy chain variable region comprising the amino acid sequence of SEQ ID NO: 87; or
(ii) a light chain variable region comprising the amino acid sequence of SEQ ID NO: 77; and a heavy chain variable region comprising the amino acid sequence of SEQ ID NO: 88; or
(iii) a light chain variable region comprising the amino acid sequence of SEQ ID NO: 77; and a heavy chain variable region comprising the amino acid sequence of SEQ ID NO: 89; or
(iv) a light chain variable region comprising the amino acid sequence of SEQ ID NO: 80; and a heavy chain variable region comprising the amino acid sequence of SEQ ID NO: 87; or
(v) a light chain variable region comprising the amino acid sequence of SEQ ID NO: 80; and a heavy chain variable region comprising the amino acid sequence of SEQ ID NO: 88; or
(vi) a light chain variable region comprising the amino acid sequence of SEQ ID NO: 80; and a heavy chain variable region comprising the amino acid sequence of SEQ ID NO: 89; or
(vii) a light chain variable region comprising the amino acid sequence of SEQ ID NO: 85; and a heavy chain variable region comprising the amino acid sequence of SEQ ID NO: 100; or
(viii) a light chain variable region comprising the amino acid sequence of SEQ ID NO: 85; and a heavy chain variable region comprising the amino acid sequence of SEQ ID NO:101; or
(ix) a light chain variable region comprising the amino acid sequence of SEQ ID NO: 86; and a heavy chain variable region comprising the amino acid sequence of SEQ ID NO: 100; or
(x) a light chain variable region comprising the amino acid sequence of SEQ ID NO: 86; and a heavy chain variable region comprising the amino acid sequence of SEQ ID NO:101.

According to certain embodiments, the anti-IL-36R antibody or antigen-binding fragment thereof comprises:
(i) a light chain comprising the amino acid sequence of SEQ ID NO: 115; and a heavy chain comprising the amino acid sequence of SEQ ID NO: 125; or
(ii) a light chain comprising the amino acid sequence of SEQ ID NO: 115; and a heavy chain comprising the amino acid sequence of SEQ ID NO: 126; or
(iii) a light chain comprising the amino acid sequence of SEQ ID NO: 115; and a heavy chain comprising the amino acid sequence of SEQ ID NO: 127; or
(iv) a light chain comprising the amino acid sequence of SEQ ID NO: 118; and a heavy chain comprising the amino acid sequence of SEQ ID NO: 125; or
(v) a light chain comprising the amino acid sequence of SEQ ID NO: 118; and a heavy chain comprising the amino acid sequence of SEQ ID NO: 126; or
(vi) a light chain comprising the amino acid sequence of SEQ ID NO: 118; and a heavy chain comprising the amino acid sequence of SEQ ID NO: 127; or
(vii) a light chain comprising the amino acid sequence of SEQ ID NO: 123; and a heavy chain comprising the amino acid sequence of SEQ ID NO: 138; or
(viii) a light chain comprising the amino acid sequence of SEQ ID NO: 123; and a heavy chain comprising the amino acid sequence of SEQ ID NO: 139; or
(ix) a light chain comprising the amino acid sequence of SEQ ID NO: 124; and a heavy chain comprising the amino acid sequence of SEQ ID NO: 138.

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

In one aspect, described and disclosed herein is an anti-IL-36R antibody that is spesolimab.

### III. PHARMACEUTICAL COMPOSITIONS, FORMULATION, DOSES, AND ADMINISTRATION

A “pharmaceutical composition” refers in this context to a liquid or powder preparation which is in such form as to permit the biological activity of the active ingredient(s) to be unequivocally effective, and which contains no additional components which are significantly toxic to the subjects to which the composition would be administered. Such compositions are sterile. A “powder” refers to a freeze-dried or lyophilized or a spray-dried pharmaceutical composition for parenteral use. The powder is reconstituted or dissolved typically in water. Lyophilization is a low temperature dehydration process which involves freezing the product, lowering pressure, then removing the ice by sublimation. Freeze drying results in a high-quality product because of the low temperature used in processing. For a well-developed lyophilized formulation, the shape and appearance of the product is maintained over time and the quality of the rehydrated product is excellent. Spray drying is another method of producing a dry powder from a liquid or slurry by rapidly drying with a hot gas and with the goal of achieving a consistent particle size distribution.

A “pharmaceutical formulation” or “formulation” refers to the process but also the product of a process in which an active drug or agent is combined with chemical substances to produce a final medicinal or drug product, the final formulation therefore refers to medicinal products such as liquids, powders, or compositions. Therefore, in one embodiment, a pharmaceutical formulation is a pharmaceutical composition.

An antibody of the invention can be incorporated into pharmaceutical compositions suitable for administration to a subject. The compounds of the invention may be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions are designed in accordance with conventional techniques as in e.g., Remington, The Science and Practice of Pharmacy, 19th Edition, Gennaro, Ed., Mack Publishing Co., Easton, PA 1995 which provides a compendium of formulation techniques as are generally known to practitioners.

A pharmaceutical composition comprising an anti-IL-36R monoclonal antibody of the present invention can be administered to a subject with a history of GPP, and/or with a confirmed history (diagnosis) of GPP based on the consensus diagnostic criteria defined by the ERASPEN Diagnostic Criteria (Website:eraspen.eu/home/rfp/diagnostic-criteria.html (access date: 9 May 2018); European Rare And Severe Psoriasis Expert Network (ERASPEN); 2018), as described herein using standard administration techniques including oral, intravenous, intraperitoneal, subcutaneous, pulmonary, transdermal, intramuscular, intranasal, buccal, sublingual, or suppository administration.

The route of administration of an antibody of the present invention may be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred. Suitable vehicles for such injections are known in the art.

The pharmaceutical composition typically must be sterile and stable under the conditions of manufacture arid storage in the container provided, including e.g., a sealed vial or syringe (e.g., to hold a unit dose form). Therefore, pharmaceutical compositions may be sterile filtered after making the formulation, or otherwise made microbiologically acceptable. A typical composition for intravenous infusion could have a volume as much as 250-1000 ml of fluid, such as sterile Ringer's solution, physiological saline, dextrose solution and Hank's solution and a therapeutically effective dose, (e.g., 1 to 100 mg/mL or more) of antibody concentration. Dose may vary depending on the type and severity of the disease. As is well known in the medical arts, dosages for any one subject depends upon many factors, including the subject's size, body surface area, age, the particular compound to be administered, sex, time and route of administration, general health, and other drugs being administered concurrently. A typical dose can be, for example, in the range of 0.001 to 1000 mg; however, doses below or above this exemplary range are envisioned, especially considering the aforementioned factors.

In one embodiment, the invention provides a pharmaceutical composition formulated in a unit dose form wherein such single dose form comprises at least 150 mg, 300 mg, 450 mg, 600 mg, or 900 mg of said anti-IL-36R antibody. In a related embodiment, the unit dose form is used in the preparation of a medicament for treating a subject having generalized pustular psoriasis (GPP). In a related embodiment, the unit dose form is a parenteral (e.g., intravenous or subcutaneous) dose form.

In a related embodiment, the invention provides a drug preparation (or “article of manufacture” described hereinafter), which comprises one, two, three or four said unit dose forms comprising 150 mg of said anti-IL-36R antibody one, two, three or four said unit dose forms comprising 300 mg of said anti-IL-36R antibody; one, two, three or four said unit dose forms comprising 450 mg of said anti-IL-36R antibody; or one, two, three or four said unit dose forms comprising 600 mg of said anti-IL-36R antibody; or one, two, three or four said unit dose forms comprising 900 mg of said anti-IL-36R antibody; or any combination thereof to achieve an efficacious dose.

In a related embodiment, the one, two, three or four said unit dose forms (e.g., in the drug preparation) are administered at 1 (qw), 2 (q2w), 4-week (q4w), 12-week (q12w) intervals. Preferably, when the unit dose forms comprises 150 mg, 300 mg, 450 mg, 600 mg, or 900 mg, of said anti-IL-36R antibody, the one-, two-, three-, or four-unit dose forms are administered at 1 (qw), 2 (q2w), 4-week (q4w) or 12-week (q12w) intervals as loading and/or subcutaneous maintenance doses.

In one aspect, the present invention relates to use of an anti-IL-36R antibody or an antigen-binding fragment thereof (as disclosed herein) in the preparation of a medicament for treating, preventing, or ameliorating GPP. In an embodiment relating to this aspect, the anti-IL-36R antibody is spesolimab.

The term “dose,” as used herein, refers to an amount of an anti-IL-36R antibody, or an antigen-binding portion thereof which is administered to a subject.

The term “dosing,” as used herein, refers to the administration of an anti-IL-36R antibody, or an antigen-binding portion thereof to achieve a therapeutic objective (e.g., treatment a subject with a history of GPP). In preferred embodiments a dose is delivered by parenteral administration, e.g., intravenously and subcutaneously.

A “dosing regimen” describes a treatment schedule for an anti-IL-36R antibody, or an antigen-binding portion thereof e.g., a treatment schedule over a prolonged period of time or throughout the course of maintenance treatment, for example, in a non-limiting embodiment, a regimen comprises a first loading dose of an anti-IL-36R antibody, or an antigen-binding portion thereof at week 0 or 1 followed by at least one maintenance dose of an anti-IL-36R antibody, or an antigen-binding portion thereof, but preferably a second, third, fourth dose, or more over the period of the maintenance therapy, wherein the dose is delivered at 2- 4-, 5-, 6-, 7-, 8-, 10- or 12-week intervals, and wherein administration of the loading dose and/or the maintenance dose is delivered parenterally, e.g., intravenously and/or intravenously.

As used herein, the terms “intravenous dose” (iv), “subcutaneous dose” (s.c.) in addition to their ordinary meanings, refer to a temporal sequence of administration of the anti-IL-36R antibody. Anti-IL-36R antibody treatment can be initiated as a subcutaneous injection to prevent GPP flares or with an intravenous dose of the anti-IL-36R antibody to treat a GPP flare. In a preferred embodiment, for the treatment of generalized pustular psoriasis (GPP), including the treatment and prevention of flares in in adults and adolescents from 12 years of age, the dosing regimen comprises a subcutaneous loading dose of 600 mg, comprising four 150 mg injections, followed by maintenance doses of 300 mg (comprising two 150 mg injections) administered subcutaneously every 4 weeks. If a subject experiences a GPP flare while receiving subcutaneous maintenance treatment with said anti-IL36R antibody, the GPP flare may be treated with intravenous anti-IL36R antibody, after which maintenance treatment can be reinitiated. Reinitiation of maintenance therapy can occur four weeks after the conclusion of the flare treatment with intravenous anti-IL-36R, wherein maintenance therapy comprises doses of 300 mg (two 150 mg injections) administered every 4 weeks, and a subcutaneous loading dose is not required.

Thus, in one embodiment a “loading dose” is the dose typically administered at the beginning of the treatment regimen (also referred to as the “baseline dose”); it may also be referred to as an “initial dose” or “induction dose.” A “maintenance dose(s)” is/are the dose or doses which are administered after the initial loading dose, which may also be referred to as a “subsequent dose(s)” which are part of the “maintenance treatment”. The loading and maintenance doses may all contain the same amount of anti-IL-36R antibody, or an antigen binding fragment thereof, but generally may differ from one another in terms of the amount of the antibody administered or the frequency of administration. In an embodiment, the loading dose is equal or larger than the maintenance dose. A “loading dose” can be a single dose or, alternatively, a set of doses. The loading dose can be intravenously or subcutaneously administered, and each dose can be a single dose or, alternatively, a set of doses, but preferably a subcutaneous dose.

The term “maintenance dose” or “treatment dose” is the amount of an anti-IL-36R antibody, or an antigen-binding portion thereof taken by a subject to maintain or continue a desired therapeutic effect. A maintenance dose is administered during the treatment or maintenance phase of therapy. In one embodiment, a maintenance dose(s) is smaller than the loading dose(s) and may be equal to each other when administered in succession. In one embodiment, the invention provides a maintenance dose of 150 mg or 300 mg of an anti-IL-36R antibody, or an antigen-binding portion thereof administered subcutaneously to a subject at 2- 4-, 5-, 6-, 7-, 8-, 10- or 12-week intervals, wherein administration of the loading dose and/or the maintenance dose is delivered subcutaneously. In one embodiment, the maintenance dose is administered every four weeks beginning 1, 2, 3, 4, 5, 6, 7, 8, 9 10, 11, 12 weeks after the last loading dose. In one embodiment, a maintenance dose is administered about 4 weeks following the initial loading dose, and at 4-week (q4w) intervals thereafter. In one embodiment, the maintenance dose is administered every twelve weeks beginning 1, 2, 3, 4, 5, 6, 7, 8, 9 10, 11, 12 weeks after the last loading dose. In one embodiment, a maintenance dose is administered about 12 weeks following the initial loading dose and at 12-week (q12w) intervals thereafter.

The terms “intravenous” or “intravenous infusion” refer to introduction of an agent into the vein of an animal or human subject over a period of time greater than approximately 15 minutes, generally between approximately 30 to 90 minutes.

The term “subcutaneous administration” refers to introduction of an agent under the skin of an animal or human subject, preferable within a pocket between the skin and underlying tissue, by relatively slow, sustained delivery from a drug receptacle. Pinching or drawing the skin up and away from underlying tissue may create the pocket.

The terms “treatment” and “therapy” and “prevention” and the like, as used herein, are meant to include therapeutic as well as prophylactic, or suppressive measures for a disease or disorder leading to any clinically desirable or “beneficial effect” or “beneficial response”, including but not limited to alleviation or relief of one or more symptoms, regression, slowing or cessation of progression of the disease or disorder. Thus, for example, the term treatment includes the administration of an agent prior to or following the onset of a symptom or symptoms of GPP disease, such a before a flare occurs thereby preventing or removing one or more signs of the disease or disorder. A beneficial response can also be exemplified by maintenance of a Generalized Pustular Psoriasis Global Assessment (GPPGA) pustulation subscore, maintenance of a GPPGA total score of 0 or 1, sustained remission of GPP symptoms, defined as a subject treated with said anti-IL-36R antibody who maintains a GPPGA score of 0 or 1 (clear or almost clear), a decrease in the occurrence of GPP flare(s), a prolongation of the time to a first GPP flare as measured by the time from baseline to the onset of said first GPP flare, or changes in the levels of biomarkers (such as micro RNAs miR-223-5p and/or miR-223-3 present in the skin or serum). As another example, the term includes the administration of an agent after clinical manifestation of GPP to combat the symptoms of the disease. Further, administration of an agent after onset and after clinical symptoms have developed where administration affects clinical parameters of the disease or disorder, such as the degree of tissue injury, whether or not the treatment leads to amelioration of the disease, comprises “treatment” or “therapy” as used herein. Moreover, as long as the compositions of the invention either alone or in combination with another therapeutic agent alleviate or ameliorate at least one symptom of GPP being treated as compared to that symptom in the absence of use of the humanized anti-IL-36R antibody composition, the result should be considered an effective treatment of the underlying disorder regardless of whether all the symptoms of the disorder are alleviated or not.

The term “prophylactically effective amount” is used to refer to an amount effective, at dosages and for periods of time necessary, to achieve the desired prophylactic result, e.g., treatment and prevention” of flares in a subject. Typically, a prophylactic dose is used in subjects with a history of generalized pustular psoriasis (GPP) and/or as diagnosed per European Rare And Severe Psoriasis Expert Network (ERASPEN) criteria prior to the onset of a GPP flare and/or prior to the onset of symptoms of GPP, which can be moderate or severe, to prevent or inhibit the occurrence of acute flares. A subject who is not undergoing a GPP flare, has a GPP Physician Global Assessment (GPPGA) total score of ≤1 and a GPPGA pustulation subscore of ≤1. In an embodiment, a maintenance dose as contemplated herein is a prophylactic dose that is used in a subject with a history of moderate to severe GPP symptoms, and/or with a confirmed history (diagnosis) of GPP based on the consensus diagnostic criteria defined by the ERASPEN Diagnostic Criteria, after the loading dose, to prevent a possible recurrence of the GPP flares in the subject or worsening of disease.

As used herein “buffer” refers to a buffered solution that resists changes in pH by the action of its acid-base conjugate components. The “pH” herein refers to the acidity or basicity of the composition at room temperature. Standard methods to measure the pH of a composition are known to the skilled in the art. Typically, measuring pH consists of calibrating the instrument, placing the electrodes in a well-mixed sample, and then reading the pH directly from the pH meter. The exemplary buffers of the present invention include acetate, citrate, histidine, succinate, phosphate, and Tris.

As used herein, the term “tonicifying agent” or “tonicity agent” or “tonicifer” refers to substances providing an osmotic pressure equivalent to that of serum in the body including salts (e.g., sodium chloride, potassium chloride, magnesium chloride) or sugars (e.g., sucrose, trehalose, sorbitol, magnesium sulfate (MgSO₄), glycerol, mannitol, or dextrose). In addition, sugars present in the solution function as a cryoprotectant for the protein which allows the drug substance to be frozen without damage. This permits shipment in the frozen form and long-term storage of the drug substance prior to the filling of drug product. The exemplary tonicifying agents of the present invention include sodium chloride, potassium chloride, magnesium chloride (salts) and/or sucrose, trehalose, sorbitol, magnesium sulfate (MgSO₄), glycerol, mannitol, or dextrose (sugars).

As used herein, the term “stabilizer” or “stabilizing agent” refers to substances contributing to the stability of the active ingredient in a pharmaceutical formulation. The exemplary stabilizing agents of the present invention include arginine, histidine, glycine, cysteine, proline, methionine, lysine, or pharmaceutically acceptable salts thereof.

As used herein, the term “surfactant” refers to substances which tend to reduce the surface tension of a liquid in which they are dissolved. The exemplary surfactants of the present invention include poloxamer 188, polysorbate 20, polysorbate 40, polysorbate 60 or polysorbate 80.

In an embodiment relating to any of the above aspects, the anti-IL-36R antibody or an antigen binding fragment thereof (disclosed herein) is present in a stable pharmaceutical formulation (as described in co-pending U.S. application Ser. No. 16/809,606, filed Mar. 5, 2020, the entire content of which is hereby incorporated herein by reference in its entirety) for administration to a mammal or subject according to any one of the aspects of the present invention.

In one embodiment, the method of treatment according to any of the aspects described herein, includes administering to the mammal or subject a therapeutic amount of a stable pharmaceutical formulation comprising from about 20 mg/mL to about 150 mg/mL of an anti-IL-36R antibody (disclosed herein), about 20 mM to about 80 mM of a pharmaceutically acceptable buffer (e.g., acetate buffer), about 100 mM to about 250 mM of a pharmaceutically acceptable tonicifying agent (e.g., sucrose), about 0 mM to about 80 mM of a pharmaceutically acceptable stabilizing agent (e.g., arginine) or a pharmaceutically acceptable salt thereof, about 0 to about 150 mM of a pharmaceutically acceptable salt (e.g., sodium chloride), and a pharmaceutically acceptable surfactant (e.g., polysorbate 20) in an amount about 0 g/L to about 1.5 g/L, wherein the subject with a history of GPP symptoms, and/or with a confirmed history (diagnosis) of GPP based on the consensus diagnostic criteria defined by the ERASPEN Diagnostic Criteria is treated, and/or the symptoms of acute GPP are prevented or ameliorated, or the skin disorder associated with GPP in the subject is treated, or skin inflammation and/or flares associated with GPP in the subject is reduced or alleviated, or the complete resolution of GPP symptoms in the subject is achieved. In a related embodiment, the stable pharmaceutical formulation is an aqueous pharmaceutical formulation. In a related embodiment, the pH of the aqueous pharmaceutical formulation is about 5 to about 7. In a related embodiment, the pharmaceutical formulation is for an intravenous administration to the mammal or subject. In a related embodiment, the pharmaceutical formulation is for a subcutaneous administration to the mammal or subject. In a related embodiment, the pharmaceutical formulation for an intravenous administration comprises an anti-IL-36R antibody in an amount of about 60 mg/mL, wherein one vial contains 450 mg. In a related embodiment, the pharmaceutical formulation for a subcutaneous administration comprises an anti-IL-36R antibody in an amount of about 150 mg/mL, wherein one pre-filled syringe contains 300 mg of antibody for subcutaneous injection

Various delivery systems are known and can be used to administer the IL-36R binding agent. Methods of introduction include but are not limited to intradermal, intramuscular, intraperitoneal, intravenous, subcutaneous, intranasal, epidural, and oral routes. The IL-36R binding agent can be administered, for example by infusion, bolus, or injection, and can be administered together with other biologically active agents such as chemotherapeutic agents. Administration can be systemic or local. In preferred embodiments, the administration is by intravenous or subcutaneous injection. Formulations for such injections may be prepared in for example prefilled syringes that may be administered once every other week.

In one aspect, the invention provides an article of manufacture comprising a subcutaneous administration device, which delivers to a subject a fixed dose of an antibody of the present invention. In some embodiments, the subcutaneous administration device is a pre-filled syringe, an autoinjector, or a large volume infusion device. For example, MyDose™ product from Roche, a single use infusion device that enables the subcutaneous administration of large quantities of liquid medication, may be used as the administration device. Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, 11 and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), YPSOMATE™ YPSOMATE 2.25™, VAIROJECT™ (Ypsomed AG, Burgdorf, Switzerland) to name only a few. Additional information relating to example delivery devices that could be used with an antibody of the present invention may be found, for example, in CH705992A2, WO2009/040602, WO2016/169748, WO2016/179713.

In specific embodiments, the IL-36R binding agent composition is administered by injection, by means of a catheter, by means of a suppository, or by means of an implant, the implant being of a porous, non-porous, or gelatinous material, including a membrane, such as a silastic membrane, or a fiber. Typically, when administering the composition, materials to which the anti-IL-36R antibody or agent does not absorb are used.

In other embodiments, the anti-IL-36R antibody or agent is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

### IV. TREATMENT OF GENERALIZED PUSTULAR PSORIASIS (GPP), INCLUDING THE TREATMENT AND PREVENTION OF FLARES IN A SUBJECT

The terms “subject” or “patient” for purposes of treatment as used herein refers to any animal classified as a mammal, including humans, domesticated and farm animals, and zoo, sports, or pet animals, such as dogs, horses, cats, cows, and the like. Preferably, the mammal is human. The human subjects suitable for treatment are adult and adolescent subjects with a history of GPP, as diagnosed per ERASPEN criteria, regardless of IL36RN mutation status, and with at least two GPP flares of moderate-to-severe intensity in the past regardless of IL36RN mutation status. Subjects also have a GPPGA total score of 0 or 1 before the initiation of prevention treatment of the anti-IL36R antibody of the present invention. “History of GPP” may also include a subject's medical history of flaring while on concomitant treatment for GPP or a history of flaring upon dose reduction or discontinuation of concomitant medications, examples of the later include other known systemic and/or topical therapies for the treatment of GPP.

The terms “maintenance therapy” or “maintenance dosing regimen”, refers to a treatment schedule for a subject with a history of GPP, which can be moderate to severe, and/or with a confirmed history (diagnosis) of GPP based on the consensus diagnostic criteria defined by the ERASPEN Diagnostic Criteria to enable them to maintain their health in a given state, e.g., reduced number or incidence of symptoms of GPP, including moderate to severe symptoms and acute GPP flares or achieving a clinical response. In one embodiment, a maintenance therapy of the invention is used for a subject with a history of GPP, and/or with a confirmed history (diagnosis) of GPP based on the consensus diagnostic criteria defined by the ERASPEN Diagnostic Criteria to enable them to maintain their health in a state which is completely free of symptoms or a reduction in symptoms associated with the disease. In one embodiment, a maintenance therapy of the invention is used for a subject or subject a history of GPP, and/or with a confirmed history (diagnosis) of GPP based on the consensus diagnostic criteria defined by the ERASPEN Diagnostic Criteria, to enable them to maintain their health in a state which is substantially free of symptoms associated with the disease. In one embodiment, a maintenance therapy of the invention is used for a subject or subject with a history of GPP to enable them to maintain their health in a state where there is a significant reduction in symptoms associated with the disease.

The term “treatment” or “maintenance treatment” as used herein, refers to a period of treatment comprising administration of an anti-IL-36R antibody, or an antigen-binding portion thereof to a subject in order to maintain a desired therapeutic effect, e.g., improved symptoms associated with acute and/or GPP.

In embodiments of the invention, maintenance treatment can occur following an incidence of GPP flare or GPP disease worsening. Maintenance treatment would be initiated once the subject after GPP flare treatment and clinical stabilization of the acute flare.

“GPP Flare treatment” is defined as treatment of a subject's experience GPP worsening with a 900 mg i.v. dose of spesolimab to treat the first GPP flare during the randomized maintenance treatment period. The criteria to receive GPP flare treatment with Open Label (OL) i.v. dose of 900 mg anti-IL36R antibody was subjects with a GPPGA score of 3 and a pustular component of GPPGA of 2 at R1 (time of first IV treatment for flare). Alternatively, treatment is indicated for with GPPGA score of 2 and a pustular component of GPPGA of 2 at R1, wherein the pustular component of GPPGA was 2 at R3/D8. After GPP flare treatment with 900 mg i.v. dose of anti-IL36R antibody (at R1/D1 or at R1/D1 and R3/D8), responders were subjects who show no flare symptoms of moderate/severe intensity with a GPPGA score of ≤3 and a pustular component score of <2. In addition, the subject had a decrease in GPPGA score by ≥1 from R1/D1. A partial response to GPP flare treatment is defined as A GPPGA score 0 or 1 was not achieved, but a reduction in GPPGA score (to <3) or GPPGA pustule sub-score (to <2) was observed after receiving the treatment with a 900 mg i.v. dose of anti-IL36R antibody. A subject can also receive a greater frequency of subcutaneous maintenance doses, for example increasing the frequency from maintenance doses of 300 mg anti-IL36R at 12-week intervals (q12w), to maintenance doses of 300 mg at 4-week intervals (q4w).

“GPP Disease worsening” is defined as worsening of clinical status or GPP skin and systemic symptoms requiring treatment intervention in the investigator's opinion.

“GPP flare” is clinically defined as an increase in GPPGA score by 2 from baseline and the pustular component of GPPGA 2. Baseline value for efficacy measurements was the last value measured before first dose of anti-IL36R antibody, e.g., spesolimab, at visit two (V2).

### V. THERAPEUTIC ENDPOINTS

Embodiments of the invention provides a means for treatment of generalized pustular psoriasis (GPP), including the treatment and prevention of flares in of a subject having with a history of GPP symptoms, and/or with a confirmed history (diagnosis) of GPP based on the consensus diagnostic criteria defined by the ERASPEN Diagnostic Criteria up to week 48 after the initiation of treatment.

In other embodiments, the invention provides methods of treatment, including methods of disease reduction in subjects with a history of GPP and improvements in the quality of life for the GPP subjects.

In other embodiments, the invention provides a method for treating certain subpopulations of subjects, including, for example, those who have failed prior therapy or have had as sub-therapeutic response, including, for example, a subject who has an inadequate response to or is intolerant to, or has a contraindication to, the standard of care. In certain embodiments, the invention is used to treat a subject with a history of GPP who has an inadequate response to or is intolerant to or has a contraindication to TNF-α inhibitors.

The methods and uses described herein provide a means of determining the efficacy of an anti-IL-36R antibody, or an antigen-binding portion thereof for treating a subject having with a history of GPP symptoms, and/or with a confirmed history (diagnosis) of GPP based on the consensus diagnostic criteria defined by the ERASPEN Diagnostic Criteria, and the use of such an anti-IL-36R antibody for the treatment of generalized pustular psoriasis (GPP), including the treatment and prevention of flares in a subject. The efficacy of the treatment of GPP may be determined using any of the measures described herein, or any measure known to those in the art, for example:
(a) A reduced risk of at least one GPP flare (defined by increase in GPPGA score by 2 from baseline and the pustular component of GPPGA 2) up to Week 48 which is greater than the placebo group.
(b) A reduced risk of worsening of Psoriasis Symptom Scale (PSS) up to Week 48 defined as a 4-point increase in total score from baseline which is greater than the placebo group.
(c) A reduced risk of worsening of Dermatology Quality of Life Index (DLQI) up to Week 48 defined as a 4-point increase in total score from baseline relative to the placebo group.

In another embodiment related to any of the above embodiments or aspects above, the administration an anti-IL-36R antibody, or an antigen-binding portion thereof results in one or more of the following efficacy endpoints as measured by the difference between GPP subjects receiving treatment compared to those in the placebo group:
(a) proportion of the subjects who did not experience a GPP flare up to week 48
(b) No PSS sub-score>1, at ≥75% of visits up to Week 48, without intake of rescue medication or investigator prescribed standard of care (SoC);
(c) A DLQI score of 0 or 1, at all visits up to Week 48, without intake of rescue medication or investigator prescribed SoC;
(d) WPAI score, over time up to Week 48
(e) GPPGA score over time up to Week 48
(f) GPPASI score over time, up to Week 48
(g) SF-36 score, over time up to Week 48
(h) Pain Visual Analog Scale (VAS) score, over time, up to Week 48
(i) EQ-5D-5L score, over time, up to Week 48
(j) JDA GPP severity score, over time, up to Week 48
(k) TPSS, over time up to Week 48
(l) PGI-S, over time up to Week 48
(m) PGI-C, over time up to Week 48
(n) Modified sustained remission, defined as subjects with a GPPGA total score of 0 or 1, and each GPPGA subscore less than or equal to 2 at all visits up to Week 48, without intake of rescue medication, or investigator prescribed SoC (added via TSAP)
(o) Modified sustained remission, defined as subjects with a GPPGA total score of 0 or 1, and each GPPGA subscore less than or equal to 2 at all visits up to Week 48, without intake of rescue medication, or investigator prescribed SoC (added via TSAP).

In another embodiment related to above embodiments or aspects above, the proportion of subjects with a response to the administration of an anti-IL-36R antibody, or an antigen-binding portion thereof is statistically significant different as compared to subjects on placebo for one or more of end points (a)-(c) and/or (a)-(o).

In one embodiment related to the above embodiments, the invention provides a method for the treatment of generalized pustular psoriasis (GPP), including the treatment and prevention of flares in a subject with a history of GPP, and/or with a confirmed history (diagnosis) of GPP based on the consensus diagnostic criteria defined by the ERASPEN Diagnostic Criteria as assessed using the Generalized Pustular Psoriasis Physician Global Assessment (GPPGA). GPPGA relies on clinical assessment of the GPP subject's skin presentation. It is a modified PGA, a physician's assessment of psoriatic lesions, which has been adapted to the evaluation of GPP subjects (Langley R G, Feldman S R, Nyirady J, et al. The 5-point Investigator's Global Assessment (IGA) Scale: A modified tool for evaluating plaque psoriasis severity in clinical trials. J Dermatol Treat 2015; 26(1):23-31). The investigator scores the erythema, pustules, and scaling of all GPP lesions from 0 to 4. Each component is graded separately, the average is calculated, and the final GPPGA is determined from this composite score. A lower score indicates a lesser severity, with 0 being clear and 1 being almost clear. GPPGA was measured at weeks 0, 1, 4, 8, 12, 16, 20, 24, 28, 32, 36, 40, 44, and 48, and 16 weeks after last dose as noted in the study (See Flow Chart of Study Activities in Examples below).

In one embodiment related to above embodiments, the invention provides a treatment of generalized pustular psoriasis (GPP), including the treatment and prevention of flares in a subject with a history of GPP, and/or with a confirmed history (diagnosis) of GPP based on the consensus diagnostic criteria defined by the ERASPEN Diagnostic Criteria as assessed using the Generalized Pustular Psoriasis Area and Severity Index (GPPASI). The GPPASI is an adaptation for GPP subjects of the PASI, an established measure of severity and area of psoriatic lesions in subjects with psoriasis (Fredriksson T, Pettersson U. Severe psoriasis-oral therapy with a new retinoid. Dermatologica 1978; 157:238-244). In the GPPASI, the induration component has been substituted with the pustule's component. It is a tool that provides a numeric scoring for a subject's overall GPP disease state, ranging from 0 to 72. It is a linear combination of percent of surface area of skin that was affected by erythema, pustules and scaling and the severity of erythema, pustules, and scaling (desquamation) over 4 body regions. GPPASI was measured at weeks 1, 4, 8, 12, 16, 20, 24, 28, 32, 36, 40, 44, and 48, and 16 weeks after last dose as noted in the study (See Flow Chart of Study Activities in Examples below).

In one embodiment related to above embodiments, the invention provides a method of treatment of generalized pustular psoriasis (GPP), including the treatment and prevention of flares in a subject with a history of GPP, and/or with a confirmed history (diagnosis) of GPP based on the consensus diagnostic criteria defined by the ERASPEN Diagnostic Criteria as assessed using the EQ-5DL questionnaire, up to week 48 after the initiation of treatment. The EQ-5D-5L self-report questionnaire was developed by the European Quality of Life Group (EuroQol Group) and is a standardized instrument for use as a measure of health outcome (EuroQol G. EuroQol—a new facility for the measurement of health-related quality of life. Health Policy 1990; 16:199-208; Herdman M, Gudex C, Lloyd A, et al. Development and preliminary testing of the new five-level version of EQ-5D (EQ-5D-5L). Qual Life Res 2011; 20:1727-1736). It contains 5 questions on different dimensions of health (e.g., mobility, self-care) and one visual analogue scale on current health. Response options include a five-level ordinal scale reporting on the five dimensions of health and a visual analogue scale reporting the subject's self-rated health status as a number between 0 and 100. MCIDs for the five-dimensional part and the visual analogue scale have been estimated as 0.074 and 7 points, respectively (Walters S J, Brazier J E. Comparison of the minimally important difference for two health state utility measures: EQ-5D and SF-6D. Qual Life Res 2005; 14:1523-1532; Pickard A S, Neary M P, Cella D. Estimation of minimally important differences in EQ-5D utility and VAS scores in cancer. Health Qual Life Outcomes 2007; 5:70). All questions refer to the current health status (“today”). The EQ-5D-5L questionnaire was measured at weeks 1, 4, 8, 12, 24, 36, and 48 as noted in the study (See Flow Chart of Study Activities in Examples below).

In one embodiment related to above embodiments, the invention provides a method for the treatment of generalized pustular psoriasis (GPP), including the treatment and prevention of flares in a subject in a subject with a history of GPP, and/or with a confirmed history (diagnosis) of GPP based on the consensus diagnostic criteria defined by the ERASPEN Diagnostic Criteria as assessed using the SF-36 questionnaire as a measure of the subjects quality of life before, during and/or after treatment. The SF-36 is a widely used instrument to measure health-related quality of life among healthy subjects and subjects with acute and chronic conditions. It consists of 36 questions (Ware J E, editor. SF-36 Health Survey: Manual and Interpretation Guide. Boston: The Health Institute, New England Medical Center; 1993). SF-36 scores can be compared across different populations of subjects and healthy subjects. Response options vary but are mostly a 5- or 3-item scale. Subscales (Physical Functioning, Role-Physical, Bodily Pain, General Health, Vitality, Social Functioning, Role-Emotional, and Mental Health) are reported individually and summarized as Physical Component Summary (PCS) and Mental Component Summary (MCS) scores (range: 0-100, with a score of 50±10 considered to reflect the US norm (Ware J E. SF-36 health survey update. Spine 2000; 25(24):3130-3139). A 3-point difference is recommended as an MCID threshold for between-groups comparisons of the PCS and MCS (Frendl D M, Ware J E. Subject-reported functional health and well-being outcomes with drug therapy: a systematic review of randomized trials using the SF-36 health survey. Med Care 2014; 52(5):439-445). The acute version of the SF-36 with a 1-week recall period was used. The SF-36 was measured at weeks 1, 12, 24, 36, and 48 (See Flow Chart of Study Activities in Examples below).

In one embodiment related to above embodiments, the invention provides a treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria as assessed using the PSS (Psoriasis Symptom Scale), wherein the efficacy of a subject's response to treatment with an anti-IL-36R antibody, or an antigen-binding portion thereof is measured as a reduced risk of worsening of symptoms as assessed by the PSS up to week 48 relative to the placebo group. The PSS is a 4-item subject-reported outcome (PRO) instrument that was developed to assess the severity of psoriasis symptoms in subjects with psoriasis (Rentz A M, Skalicky A M, Burslem K, et al. The content validity of the PSS in subjects with plaque psoriasis. J Patient Rep Outcomes 2017; 1:4). When completing this questionnaire, patients report on symptoms experienced from their Generalized Pustular Psoriasis. The symptoms included are pain, redness, itching, and burning. Current symptom severity is assessed using a 5-point scale ranging from 0 (none) to 4 (very severe). The symptom scores are added to an unweighted total score (range: 0 to 16). The PSS instrument was measured at weeks 1, 4, 8, 12, 16, 20, 24, 28, 32, 36, 40, 44, and 48, as noted in the study (See Flow Chart of Study Activities in Examples below).

In one embodiment related to above embodiments, the invention provides a method for the treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria wherein the efficacy of a subject's response to treatment with an anti-IL-36R antibody, or an antigen-binding portion thereof is measured using the WPAI-GPP (Work Productivity and Activity Impairment Questionnaire, GPP specific version). The WPAI-GPP is the GPP-specific version of a frequently used questionnaire with 6 questions assessing social functioning, regarding absenteeism, presenteeism and daily activity impairment. Response options include the number of hours worked and missed (due to GPP and due to unrelated reasons) and a numeric rating scale (0-10) assessing the impairment of work and daily activities due to GPP. The recall period is 7 days. The WPAI-GPP instrument was measured at weeks 1, 12, 36, and 48, as noted in the study (See Flow Chart of Study Activities in Examples below).

In one embodiment related to above embodiments, the invention provides a method for the treatment of generalized pustular psoriasis (GPP), including the treatment and prevention of flares in a subject with a history of GPP symptoms, and/or with a confirmed history (diagnosis) of GPP based on the consensus diagnostic criteria defined by the ERASPEN Diagnostic Criteria), wherein the efficacy of a subject's response to treatment with an anti-IL-36R antibody, or an antigen-binding portion thereof is measured using the Visual Analog Scale for Pain (VAS Pain). The VAS Pain is a unidimensional measure of pain intensity (Hawker G A, Mian S, Kendzerska T, et al. Measures of adult pain: Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP Arthritis Care Res (Hoboken) 2011; 63(Suppl 11):S240-S252). It is a continuous scale comprised of a horizontal or vertical line, usually 10 centimeters (100 mm) in length, anchored by word descriptors at each end (‘no pain’, ‘very severe pain’). The pain VAS was self-completed by the respondent. The respondent was asked to place a vertical (1) mark on the horizontal line to indicate the severity of pain. Using a ruler, the score was determined by measuring the distance (mm) on the 10-cm line between the “no pain” anchor and the patient's mark, providing a range of scores from 0 to 100. A higher score indicates greater pain intensity. Pain VAS was measured at weeks 1, 4, 8, 12, 16, 20, 24, 28, 32, 36, 40, 44, and 48, as noted in the study (See Flow Chart of Study Activities in Examples below).

In another embodiment related to above embodiments, the invention provides a method of treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria), wherein the efficacy of a subject's response to treatment with an anti-IL-36R antibody, or an antigen-binding portion thereof is measured as reduced risk of worsening of a Dermatology Life Quality Index (DLQI) score of a subject, wherein a high score (i.e., the quality of life is impaired), and a low(er) score (i.e., the quality of life not impacted or low impact). A reduced risk is such that the DLQI score is maintained at a baseline level at the start of treatment and/or decreased from a higher score to a lower score, up to Week 48 after the initiation of treatment, defined as a 4-point increase in total score from baseline. The DLQI is a patient-administered, ten-question, quality of life questionnaire that covers six domains including symptoms and feelings, daily activities, leisure, work and school, personal relationships, and treatment (Finlay A Y, Khan G K. Dermatology Life Quality Index (DLQI)—a simple practical measure for routine clinical use. Joint Ann Mtg of the British Association of Dermatologists and the Canadian Dermatology Association, Oxford, 6-10 Jul. 1993. Clin Exp Dermatol 1994; 19:210-216). The DLQI has a one-week recall period. Response categories include “not relevant” (score of 0), “not at all” (score of 0), “a little” (score of 1), “a lot” (score of 2) and “very much” (score of 3). Question 7 is a “yes”/“no” question where “yes” is scored as 3. The DLQI total score is calculated by summing the scores of each question resulting in a range of 0 to 30. The higher the score, the more the quality of life is impaired. The DLQI Instrument was measured at weeks 1, 4, 8, 12, 24, 36, and 48, as noted in the study (See Flow Chart of Study Activities in Examples below).

In another embodiment related to above embodiments, the invention provides a method for the treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria, wherein the efficacy of a subject's response to treatment with an anti-IL-36R antibody, or an antigen-binding portion thereof is measured by a JDA GPP Severity Score. The JDA GPP severity score was established by the Japanese Dermatological Association (JDA), and it consists of assessment of skin symptoms and systemic symptoms/assessment of test findings. Each item of skin symptom (area of erythema [total], area of erythema with pustules and area of edema) was rated from 0 to 3, and systemic symptoms/assessment of laboratory findings (fever, WBC count, CRP, and serum albumin) from 0 to 2 (Cosentyx for subcutaneous injection 150 mg syringe, subcutaneous injection 150 mg (secukinumab) (Novartis), Rx only: review report (Nov. 12, 2015). Website:pmda.go.jp/files/000216877.pdf (access date: 9 May 2018); Pharmaceuticals and Medical Devices Agency (PMDA); 2015). The total score of JDA severity index for GPP was assigned a score of 0-17 (0=best, 17=worst), wherein clinical improvement is defined as a reduction in risk of worsening of total JDA severity index for GPP score of up to Week 48 after the initiation of treatment, defined as an increase in total score from baseline. The JDA GPP severity score was measured at weeks 1, 4, 8, 12, 16, 20, 24, 28, 32, 36, 40, 44, and 48, and 16 weeks after last dose as noted in the study (See Flow Chart of Study Activities in Examples below).

In another embodiment related to above embodiments, the invention provides a method for the treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria, wherein the efficacy of a subject's response to treatment with an anti-IL-36R antibody, or an antigen-binding portion thereof is measured by CGI-Improvement as per JDA Severity Index. The CGI-I is an observer-rated scale which measures illness global improvement (CGI-I—as per JDA severity index guidelines) (Id.) It was categorized as “worsened”, “no change”, “minimally improved”, “much improved” or “very much improved”. The CGI-I test was at weeks 1, 4, 8, 12, 16, 20, 24, 28, 32, 36, 40, 44, and 48, and 16 weeks after last dose as noted in the study (See Flow Chart of Study Activities in Examples below).

In another embodiment related to above embodiments, the invention provides a method for the treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria, wherein the efficacy of a subject's response to treatment with an anti-IL-36R antibody, or an antigen-binding portion thereof is measured by a Target Plaque Severity Score (TPSS). The Target Plaque Severity Score (TPSS) is measured in patients with concurrent plaque psoriasis if corresponding target lesion area were identified and the severity threshold was met as described below. A target lesion of at least 9 cm2 with a TPSS≥5 and an induration sub-score 2 was selected by the investigator at baseline. The severity of erythema, scaling, and induration (plaque thickness) of this selected target lesion was assessed by the investigator at baseline and subsequent visits on a 5-point scale ranging from 0=none to 4=very marked. TPSS was measured at weeks 1, 4, 16, 20, 24, and 48 as noted in the study (See Flow Chart of Study Activities in Examples below).

In another embodiment related to above embodiments, the invention provides a method for the treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria, wherein the efficacy of a subject's response to treatment with an anti-IL-36R antibody, or an antigen-binding portion thereof is measured by the PGI-S (Patient Global Impression of Severity). The PGI-S is a single item self-assessment of current disease severity. Patients were asked to rate the severity of their Generalized Pustular Psoriasis (GPP) on a 5-point scale ranging from “normal” to “extremely severe”. The PGI-S was measured at weeks 1, 4, 8, 12, 24, 36, and 48 as noted in the study (See Flow Chart of Study Activities in Examples below).

In another embodiment related to above embodiments, the invention provides a method for the treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria, wherein the efficacy of a subject's response to treatment with an anti-IL-36R antibody, or an antigen-binding portion thereof is measured by PGI-C (Patient Global Impression of Change). The PGI-C is a single item self-assessment of how patients feel their Generalized Pustular Psoriasis (GPP) has changed since start of the trial. Patients were asked to rate the perceived change on a 7-point scale ranging from “very much improved” to “very much worse”. The PGI-C was measured at weeks 1, 4, 8, 12, 24, 36, and 48 as noted in the study (See Flow Chart of Study Activities in Examples below).

In another embodiment related to above embodiments, the proportion of subjects with a response to the administration of anti-IL36R is significantly different as compared to subjects being treated with a placebo for one or more of end points.

In one aspect, the present invention relates to a method of prolongation of the time to the first GPP flare in a subject with a history of generalized pustular psoriasis (GPP) and/or as diagnosed per European Rare And Severe Psoriasis Expert Network (ERASPEN) criteria, up to Week 48, wherein GPP flare is defined by a GPPGA pustulation subscore of >2 and an increase in GPPGA total score by 2 from baseline in a subject with a history of GPP, as diagnosed per ERASPEN criteria, comprising administration to said subject one or more dose(s) of the anti-IL-36R antibody according to any of aspects of the above embodiments. In one embodiment, said method comprises the administration to the subject an effective amount of the anti-IL-36R antibody in one or more subcutaneous doses delivered as a first loading dose of 300 mg-600 mg of the anti-IL-36R antibody, followed by maintenance doses of 150 mg-600 mg of the anti-IL-36R antibody.

In one aspect, the present invention relates to a method of achieving prevention of GPP flares of moderate-to-severe intensity in a subject with a history of GPP, as diagnosed per ERASPEN criteria, following treated with the anti-IL-36R antibody according to any of aspects or the above embodiments; wherein the GPP symptoms comprise inflammatory lesions, abscesses, GPP associated inflammation (erythema, induration, open ulcers), and/or GPP associated pain.

In one embodiment related to any of aspects above or the related embodiment(s), at least 10%, 20%, 30%, 40%, 50%, 60%, 70% or 80% of the subjects show clinical improvement as measured as the time to GPP flare (defined by a GPPGA pustulation subscore of >2 and an increase in GPPGA total score by 2 from baseline) at Week 4, 8, 12, 16, 20, 24, 28, 32, 36, 40, 44, or 48 of the treatment.

In one embodiment related to any of aspects above or the related embodiment(s), at least 10%, 20%, 30%, 40%, 50%, 60%, 70% or 80% of the subjects show improvement expressed as decrease in the occurrence of at least one GPP flare at Week 4, 8, 12, 16, 20, 24, 28, 32, 36, 40, 44, or 48 of the treatment.

In one embodiment related to any of aspects above or the related embodiment(s) at least 10%, 20%, 30%, 40%, 50%, 60%, 70% or 80% of the subjects show improvement as measured by the time to the first worsening of PSS (defined as a 4-point increase in total score from baseline) at Week 4, 8, 12, 16, 20, 24, 28, 32, 36, 40, 44, or 48 of the treatment.

In one embodiment related to any of aspects above or the related embodiment(s) at least 10%, 20%, 30%, 40%, 50%, 60%, 70% or 80% of the subjects show improvement as measured by the time to the first worsening of Dermatology Quality of Life Index (DLQI) (defined as a 4-point increase in total score from baseline) at Week 4, 8, 12, 16, 20, 24, 28, 32, 36, 40, 44, or 48 of the treatment.

In one embodiment related to any of aspects above or the related embodiment(s) at least 10%, 20%, 30%, 40%, 50%, 60%, 70% or 80% of the subjects show complete remission at Week 4, 8, 12, 16, 20, 24, 28, 32, 36, 40, 44, or 48 of the treatment.

In embodiments of the invention related to any of the aspects above include evaluation of biomarkers to assess changes pre and post treatment of generalized pustular psoriasis (GPP), including the treatment and prevention of flares in a subject of a subject with a history of GPP symptoms, and/or with a confirmed history (diagnosis) of GPP based on the consensus diagnostic criteria defined by the ERASPEN Diagnostic Criteria. Serum and skin biopsies were collected at time points indicated in the flow chart(s) for the analysis of biomarkers.

In embodiments of the inventions skin biopsies were collected prior to the administration of the study drug, and at time points during the maintenance treatment to assess changes in gene and protein expression levels pre and post treatment with spesolimab. Histological assessment of skin thickness, appearance of epidermis and dermis were evaluated pre and post treatment and summarized in a global histopathologic score for each subject per timepoint. The markers assessed by immunohistochemistry included but were not limited to K16, Ki67, S100 A7, lipocalin 2, ß defensin 2, CD3+T lymphocytes, CD11+ dendritic cells, IL-17C, IL8, NFκβ, TNFα, IL36γ, IL36R and neutrophil elastase. Expression of several of these markers were assessed on dermal and epidermal tissue. In a related embodiment, serum was collected to assess changes in protein levels of select IL-36 pathway and GPP disease specific markers pre and post treatment with spesolimab. The biomarker assay analysis of samples is performed in a staged approach.

In embodiments of the inventions skin biopsies are collected prior to the administration of the study drug, and at time points during the maintenance treatment to assess changes in microRNA expression levels pre and post treatment with spesolimab. MicroRNAs (miRNAs) are small non-coding RNA molecules able to post-transcriptionally modulate gene expression. The microRNAs assessed by small RNA sequencing, included but were not limited to miR-223-3p, miR-223-5p, miR-1304-3p, miR-485-5p, or miR-337-5p, wherein downregulation or upregulation post-treatment as compared to pre-treatment is associated with beneficial response to treatment. The results of the microRNA analyses of samples of non-lesional and lesional skin, as well as serum taken from GPP patients pre- and post-spesolimab treatment is also associated with clinical outcomes, such as GPPASI and GPPGA scores.

In embodiments of the invention, blood sample are used to evaluate known GPP mutations causative for GPP, e.g., IL36RN, CARD14 and AP1S3 genes. Subsequently their potential influence on the activity of the disease and/or efficacy of the drug within the context of the clinical trial was evaluated

### VI. MODE OF ACTION

An anti-IL-36R antibody of the present invention is a humanized antagonistic monoclonal IgG1 antibody that blocks human IL36R signaling. Binding of the anti-IL36R antibody, spesolimab to IL36R prevents the subsequent activation of IL36R by cognate its ligands (IL36 α, β and γ) and downstream activation of pro-inflammatory and pro-fibrotic pathways. IL-36R signaling is differentiated from TNF-α, integrin and IL-23 inhibitory pathways by directly and simultaneously blocking both inflammatory and pro-fibrotic pathways. Genetic human studies have established a strong link between IL36R signaling and skin inflammation. IL-36R is also known as IL-1 RL2 and IL-1 Rrp2. It has been reported that agonistic IL-36 ligands (α, β, or γ) initiate the signaling cascade by engaging the IL-36 receptor which then forms a heterodimer with the IL-1 receptor accessory protein (IL-1 RAcP). IL-36 antagonist ligands (IL-36RA/IL1F5, IL-38/ILF10) inhibit the signaling cascade.

As provided herein, an anti-IL-36R antibody of the present invention has been evaluated and proved to be effective in treating subjects with acute Generalized Pustular Psoriasis (GPP), a severe inflammatory skin disease driven by uncontrolled IL36 activity. In EFFISAYIL™-1, a randomized, double-blind, placebo-controlled clinical trial demonstrated the clinical efficacy and safety of the anti-IL36R antibody, SPEVIGO®, in adult patients with flares of Generalized Pustular Psoriasis (GPP), as diagnosed per European Rare and Severe Psoriasis Expert Network (ERASPEN) criteria. At Week 1, there was a statistically significant difference in the proportion of patients achieving a GPPGA pustulation sub score of 0 (indicating no visible pustules) and GPPGA total score of 0 or 1 (clear or almost clear skin) in the SPEVIGO® treatment arm compared with placebo. The results the clinical trial EFFISAYIL™-1 (NCT03782792) and EFFISAYIL™-ON (NCT03886246), the open label extension, are herein incorporated by reference in their entirety.

### VII. ARTICLES OF MANUFACTURE

In another aspect, an article of manufacture containing materials useful for the treatment of the disorders described above is included. The article of manufacture comprises a container and a label. Suitable containers include, for example, bottles, vials, syringes, and test tubes (e.g., to hold the unit dose forms). The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the humanized anti-IL-36R antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The term “package insert” is used to refer to instructions customarily included in commercial packages of therapeutic products, that contain information about the indications, usage, administration, contraindications and/or warnings concerning the use of such therapeutic products.

The invention is further described in the following examples, which are not intended to limit the scope of the invention. Those skilled in the art will recognize or be able to ascertain numerous equivalents to the specific substances and procedures described herein.

In one embodiment, the invention provides a pharmaceutical composition formulated in a unit dose form wherein such single dose form comprises at least 150, mg 300 mg, 450 mg, 600 mg, of said anti-IL-36R antibody. In a related embodiment, the unit dose form is used in the preparation of a medicament for treatment of a subject having generalized pustular psoriasis (GPP), including the treatment and prevention of flares in a subject with a history of GPP symptoms and/or with a confirmed history (diagnosis) of GPP based on the consensus diagnostic criteria defined by the ERASPEN Diagnostic Criteria. In a related embodiment, the unit dose form is used in the preparation of a medicament for treatment of a subject having generalized pustular psoriasis (GPP) in a subject with a history of GPP when not experiencing a flare. In a related embodiment, the unit dose form is used in the preparation of a medicament for reducing the incidence, recurrence of, and/or frequency of generalized pustular psoriasis (GPP) flares in a subject with a history of GPP symptoms and/or with a confirmed history (diagnosis) of GPP based on the consensus diagnostic criteria defined by the ERASPEN Diagnostic Criteria.

In a related embodiment to any of the aforementioned embodiments, the unit dose form is a parenteral (e.g., intravenous or subcutaneous) dose form, preferably in a subcutaneous dose form.

In a related embodiment, the invention provides a drug preparation (or “article of manufacture” described hereinafter), which comprises one, two, three or four said unit dose forms comprising 150 mg of said anti-IL-36R antibody; one, two, three or four said unit dose forms comprising 300 mg of said anti-IL-36R antibody; or one, two, three or four said unit dose forms comprising 450 mg of said anti-IL-36R antibody; or one, two, three or four said unit dose forms comprising 600 mg of said anti-IL-36R antibody; or any combination thereof to achieve an efficacious dose.

In a related embodiment, the one, two, three or four said unit dose forms (e.g., in the drug preparation) are administered as a first loading dose of 300 mg-600 mg of an anti-IL-36R antibody; followed by maintenance doses of 150 mg-600 mg of said anti-IL-36R antibody administered to the subject at 4 week (q4w) or 12 week (q12w) intervals for 8-48 weeks following the last maintenance dose.

### EXAMPLES

Standard treatment guidance for GPP often follows that of plaque psoriasis, despite limited evidence on the efficacy of anti-psoriatic drugs, including biologics, in GPP (Gooderham M J, Van Voorhees A S, Lebwohl M G. An update on generalized pustular psoriasis. Expert Rev Clin Immunol. 2019; 15(9):907-19). In Japan, Taiwan, and Thailand, several biologic agents targeting pro-inflammatory pathways associated with GPP have been approved for patient use, but the rarity of the disease means that their approval is based on a limited number of open-label clinical trials with small numbers of participants (Fujita H, Terui T, Hayama K, et al. Japanese guidelines for the management and treatment of generalized pustular psoriasis: the new pathogenesis and treatment of GPP. J Dermatol. 2018; 45(11):1235-70; Takeichi T, Akiyama M. Generalized pustular psoriasis: clinical management and update on 358 Dermatol Ther (Heidelb) (2023) 13:347-359 autoinflammatory aspects. Am J Clin Dermatol. 2020; 21(2):227-36; Thailand Food and Drug Administration. LUMICEF Summary of Product Characteristics 2019; Taiwan Center for Drug Evaluation. Lumicef subcutaneous injection 210 mg syringe 201; Morita A, Kotowsky N, Gao R, Shimizu R, Okubo Y. Patient characteristics and burden of disease in Japanese patients with generalized pustular psoriasis: results from the Medical Data Vision claims database. J Dermatol. 2021; 48(10):1463-73)

In an open-label, proof-of-concept study (NCT02978690), patients experiencing a GPP flare became clear or almost clear of GPP by Week 4 post treatment with a single dose of spesolimab (a humanized anti-interleukin-36 receptor monoclonal antibody) (Bachelez H, Choon S E, Marrakchi S, Burden A D, Tsai T F, Morita A, et al. Inhibition of the interleukin-36 pathway for the treatment of generalized pustular psoriasis. N Engl J Med. 2019; 380(10): 981-3). After this, the Effisayil™1 study (NCT03782792) was the first randomized clinical trial to investigate a targeted treatment for GPP and reported that adult patients with a GPP flare treated with spesolimab achieved rapid pustular and skin clearance (Bachelez H, Choon S E, Marrakchi S, Burden A D, Tsai T F, Morita A, et al. Trial of spesolimab for generalized pustular psoriasis. N Engl J Med. 2021; 385(26):2431-40). The results from this study were integral to the approval of spesolimab by the US FDA as a first treatment option for GPP flares. The relapsing nature of GPP (recurrent flares or persistent disease with intermittent flares) highlights the need to develop treatments to prevent flares (Navarini A A, Burden A D, Capon F, Mrowietz U, Puig L, Ko\ddot{ } ks S, et al. European consensus statement on phenotypes of pustular psoriasis. J Eur Acad Dermatol Venereol. 2017; 31(11):1792-9), with a recent survey revealing that among dermatologists whose patients experience frequent flares, 67% felt that currently available treatments fail to adequately prevent new flares (Strober B, Kotowsky N, et al. Unmet medical needs in the treatment and management of generalized pustular psoriasis flares: evidence from a survey of Corrona Registry Dermatologists. Dermatol Ther (Heidelb). 2021; 11(2):529-41).

Example 1: Study Design: Effisayil™ 2 is a multi-center, randomized, parallel group, double blind, placebo controlled, Phase IIb dose finding study which evaluated the efficacy and safety of spesolimab compared with placebo in preventing generalized pustular psoriasis (GPP) flares in subjects with history of Generalized Pustular Psoriasis GPP: Effisayil™ 2 is the first study to investigate the use of an antibody against the interleukin-36 receptor for the prevention of the occurrence of GPP flares, a key step for the efficacy assessment of spesolimab in an intermittently, repeatedly flaring disease, and determined the optimal dosing regimen of spesolimab SC maintenance treatment.

Objectives: EFFASIYL™-2 (NCT04399837) evaluated the efficacy and safety of spesolimab for subcutaneous administration in adult and adolescent subjects with a history of GPP, as diagnosed per ERASPEN criteria, regardless of IL36RN mutation status, and with at least two GPP flares of moderate-to-severe intensity in the past. Subjects were required to discontinue systemic and topical therapy for GPP prior to or at randomisation. These subjects had a history of flaring while on concomitant treatment for GPP or a history of flaring upon dose reduction or discontinuation of these concomitant medications.

The primary objective was to demonstrate a non-flat curve, characterize the dose response relationships of 3 subcutaneous dosing regimens of spesolimab (with each regimen comprising a single loading dose and a separate maintenance subcutaneous dosing regimen versus placebo, in subjects with a history of GPP (per ERASPEN criteria), who presented (at screening and at randomization) with a GPPGA score of 0 or 1 (clear, or almost clear), on the primary endpoint of the time to the first GPP flare onset up to Week 48.

The primary endpoint of the study is the time to the first GPP flare up to Week 48 (defined by a GPPGA pustulation subscore of >2 and an increase in GPPGA total score by ≥2 from baseline). The key secondary endpoint of the study is the occurrence of at least one GPP flare up to Week 48, i.e., how long it takes subjects to have a GPP flare, while they are being given spesolimab or placebo. Additional secondary endpoints at Week 48 were the time to the first worsening of PSS and Dermatology Quality of Life Index (DLQI) defined as a 4-point increase in total score from baseline.

Overall Design: The randomized multicenter, parallel group, double-blind, placebo-controlled Phase IIb study comprised 3 active doses compared to placebo in adolescents from 12 years to less than 18 years of age and adult subjects with history of GPP and currently presenting (at screening and at randomization) with a GPPGA score of 0 or 1 (clear or almost clear). The active treatment arms consisted of active loading dose and active maintenance treatment.

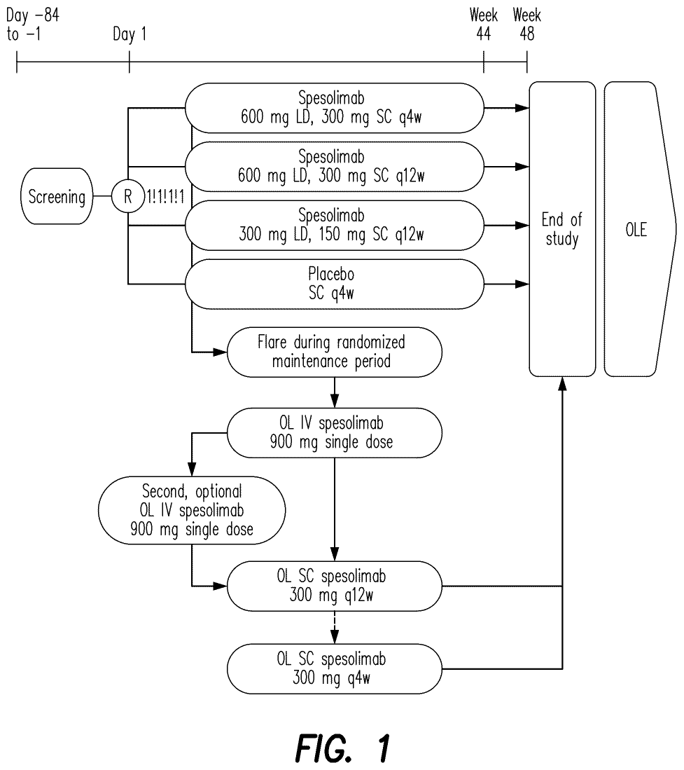

A total of 123 eligible subjects with generalized pustular psoriasis (GPP) were randomized in a 1:1:1:1 ratio to placebo, low, medium, or high treatment arms. (FIG. 1)

TABLE 2
Treatment arms in EFFASIYL ™ 2

| Treatment | | |
|---|---|---|
| Groups | Loading dose | Maintenance doses |
| Spesolimab | 600 mg subcutaneously | 300 mg subcutaneously |
| (High) | | every 4 weeks |
| Spesolimab | 600 mg subcutaneously | 300 mg subcutaneously |
| (Medium) | | every 12 weeks |
| Spesolimab | 300 mg subcutaneously | 150 mg subcutaneously |
| (Low) | | every 12 weeks |
| Placebo | subcutaneous treatment | subcutaneous treatment |
| | | every 4 weeks |

All subjects received the first dose of study medication on Day 1. Each randomized subject received 4 injections (loading dose) at Week 1/Day 1 followed by 2 injections (maintenance treatment) at subsequent visits until Week 44 if no flare occurred. The maintenance treatment period ended at Week 48.

The primary statistical analysis of the trial was performed once all randomized subjects had either completed or early discontinued from the 48-week maintenance treatment period.

Inclusion Criteria: The main diagnosis for trial entry included those individuals who were ≥12 years and ≤75 years old with a confirmed history (diagnosis) of GPP based on the consensus diagnostic criteria defined by the ERASPEN and the subjects must have had previous evidence (for past GPP flares) of either fever, and/or asthenia, and/or myalgia, and/or elevated C-reactive protein, and/or leukocytosis with peripheral blood neutrophilia (above ULN). The ERASPEN diagnosis criteria were: Primary, sterile, macroscopically visible pustules on non-acral skin (excluding cases where pustulation was restricted to psoriatic plaques) i) with or without systemic inflammation; ii) with or without plaque-type psoriasis, and iii) either relapsing (>1 episode) or persistent (>3 months).

Each subject met all the following inclusion criteria to be included into the trial:
Subjects with a known and documented history of GPP per ERASPEN criteria (regardless of IL36RN mutation status, with at least 2 presentations of GPP flares with fresh pustulation (new appearance or worsening) in the past.
Subjects with a GPPGA score of 0 or 1 at screening and randomization.
Subjects who were not on concomitant GPP treatment at time of randomization (V2) must have had at least two presentations of GPP flare in the past year, at least one of which had evidence of either fever and/or elevated CRP and/or elevated WBC, and/or asthenia and/or myalgia.
Subjects who were not on concomitant GPP treatment at time of randomization (V2) but who were on concomitant GPP treatment until shortly before randomization (V2) (512 weeks before randomization), these subjects must have a history of flaring while on concomitant treatment for GPP or in case of dose reduction or discontinuation of their concomitant medication.
Subjects who were on concomitant treatment regimen with retinoids and/or methotrexate and/or cyclosporine must stop at the day of randomization (V2). These subjects must have had a history of flaring while on concomitant treatment for GPP or in case of dose reduction or discontinuation of these concomitant medications.
Male or female subjects aged 12 to 75 years at screening. For all subjects, a minimum weight of 40 kg was required.
Signed and dated written informed consent and assent in accordance with ICH-GCP and local legislation prior to admission in the trial.
women of childbearing potential (WOCBP)1 were ready and able to use highly effective methods of birth control per ICH M3 (R2) that result in a low failure rate of less than 1% per year when used consistently and correctly. A list of contraception methods meeting these criteria was provided in the CTP as well as in the subject, parent(s) (or subject's legal guardian) information.

### Exclusion Criteria:

Subjects with SAPHO (Synovitis acne-pustulosis-hyperostosis-osteitis) syndrome.
Subjects with primary erythrodermic psoriasis vulgaris.
Severe, progressive, or uncontrolled hepatic disease, defined as >3-fold Upper Limit of Normal (ULN) elevation in AST or ALT or alkaline phosphatase, or >2-fold ULN elevation in total bilirubin.
Treatment with: a. Any restricted medication as specified in the CTP, or any drug considered likely to interfere with the safe conduct of the study, as assessed by the investigator. B. Any prior exposure to spesolimab or another IL36R inhibitor biologic.
Increased risk of infectious complications (e.g., recent pyogenic infection, any congenital or acquired immunodeficiency (e.g., HIV), past organ or stem cell transplantation), as assessed by the investigator
Relevant chronic or acute infections including active tuberculosis, human immunodeficiency virus (HIV) infection or viral hepatitis at the time of randomization. Subjects may have been re-screened if the subject was treated and was cured from the acute infection.
Active or Latent TB.
History of allergy/hypersensitivity to the systemically administered trial medication agent or its excipients.
Any documented active or suspected malignancy or history of malignancy within 5 years prior to screening, except appropriately treated basal or squamous cell carcinoma of the skin or in situ carcinoma of uterine cervix.
Currently enrolled in another investigational device or drug study, or less than 30 days since ending another investigational device or drug study(s) or receiving other investigational treatment(s). Exception: Subjects in the 1368-0013 study who are in the screening period and who were not randomized in the 1368-0013 trial due to the study meeting target number of randomized subjects or who did not qualify to be randomized into the 1368-0013 study may have been enrolled in the 1368-0027 study if they meet all inclusion/exclusion criteria.
Women who are pregnant, nursing, or who plan to become pregnant while in the trial. Women who stop nursing before the study drug administration do not need to be excluded from participating; they should refrain from breastfeeding for 16 weeks after the last study drug administration.
Major surgery (major according to the investigator's assessment) performed within 12 weeks prior to receiving first dose of study drug or planned during the study, e.g., hip replacement, aneurysm removal, stomach ligation, as assessed by the investigator.
Evidence of a current or previous disease, medical condition (including chronic alcohol or drug abuse or congestive heart disease or any condition) other than GPP, surgical procedure, psychiatric or social problems, medical examination finding (including vital signs and electrocardiogram (ECG)), or laboratory value at the screening outside the reference range that in the opinion of the investigator was clinically significant and would make the study participant unreliable to adhere to the protocol, comply with all study visits/procedures or to complete the trial, Subjects with SAPHO (Synovitis, acne, pustulosis, hyperostosis, osteitis) syndrome.
Subjects with primary erythrodermic psoriasis vulgaris.
Severe, progressive, or uncontrolled hepatic disease, defined as >3-fold Upper Limit of Normal (ULN) elevation in AST or ALT or alkaline phosphatase, or >2-fold ULN elevation in total bilirubin.
Treatment with: a. Any restricted medication as specified in the CTP, or any drug considered likely to interfere with the safe conduct of the study, as assessed by the investigator. B. Any prior exposure to spesolimab or another IL36R inhibitor biologic.

### Example 2: Treatments Administered

The Spesolimab molecule is an anti-human IL-36 receptor monoclonal antibody heterodimer with a molecular weight of approximately 146 kDa. Spesolimab solution for injection (s.c. administration) was formulated at 150 mg/mL presented in a 1 mL pre-filled syringe (150 mg/syringe). Spesolimab solution for infusion (i.v. administration) was formulated at 60 mg/mL presented in a 10 mL vial with a nominal fill volume of 7.5 mL (450 mg).

TABLE 3
Test Product: Spesolimab (Spesolimab)
Anti-IL-36R antibody (Spesolimab) for Maintenance Treatment - including loading dose

| | |
|---|---|
| Substance: | anti-IL-36R antibody (Spesolimab) |
| Pharmaceutical formulation: | Solution for injection |
| Unit strength: | Spesolimab 150 mg/PFS (150 mg/mL), 1 mL fill volume |
| Posology: | Arm 1: 600 mg total loading dose at Week 1/Day1 followed |
| | by maintenance treatment 300 mg s.c. q4 weeks |
| | Arm 2: 600 mg total loading dose at Week 1/Day1 followed |
| | by maintenance treatment 300 mg s.c. q12 weeks |
| | Arm 3: 300 mg total loading dose at Week 1/Day1 followed |
| | by maintenance treatment 150 mg s.c. q12 weeks |
| Mode of Administration | s.c. injection |

Anti-IL-36R antibody (Spesolimab) for GPP Flare

| | |
|---|---|
| Substance: | anti-IL-36R antibody (Spesolimab) |
| Pharmaceutical formulation: | Solution for infusion |
| Unit strength: | Spesolimab 450 mg/vial (60 mg/mL), 7.5 mL fill volume |
| Posology: | 900 mg i.v. dose |
| Mode of Administration: | i.v. infusion |

Placebo

| | |
|---|---|
| Substance: | Placebo to match Solution for Injection (spesolimab) |
| Pharmaceutical formulation: | Solution for injection |
| Unit strength: | Placebo to match Solution for Injection (spesolimab), 1 mL |
| | PFS |
| Posology: | Arm 4: Loading dose Placebo at Week 1/Day 1 followed by |
| | maintenance treatment placebo |
| Mode of Administration: | s.c. injection |

### Doses and Dosing Regimens:

The aim of the current trial Effisayil™ 2 was to provide dose-ranging data for 3 subcutaneous dosing regimens of spesolimab with the proposed doses of 300 mg s.c. q4w and 300 mg s.c. q12w after a loading dose of 600 mg each, and 150 mg q12w after a loading dose of 300 mg. These regimens were selected to assess a wide range in exposure to thoroughly evaluate the exposure-response relationship of spesolimab in GPP subjects.

If a subject experienced a GPP flare (increase in GPPGA score by 2 from baseline and the pustular component of GPPGA 2) during the randomized maintenance treatment period, a GPP flare treatment with i.v. open label dose of 900 mg spesolimab was administered at day 1 (R1/D1). A subject qualified to receive another dose of i.v. open label 900 mg spesolimab at day 8 (R3/D8) if protocol specified criteria were met. The 900 mg spesolimab i.v. dose was selected based on positive results of previous trials in subjects and healthy volunteers showing that spesolimab was safe and tolerable, and within the safety limits for subjects with a lower weight.

To maintain the blinding for treatment, all subjects received the blinded treatments every 4 weeks.

TABLE 4
Loading Dose & Maintenance Treatments of anti-IL36R antibody, Spesolimab
Randomized Maintenance Treatment Period

| | Loading Dose (Blinded) | Maintenance Treatment (Blinded) |
|---|---|---|
| Arm: | Week 1/Day 1 | Week 4 through Week 44 |
| Arm 1: 300 mg | 4 × Spesolimab 150 | 2 × Spesolimab 150 mg/PFS s.c. q4 |
| Spesolimab q 4 weeks | mg/PFS (600 mg | weeks (300 mg total/dose) |
| | total/dose) | |

Maintenance Treatment Period

| | | |
|---|---|---|
| Arm 2: | 4 × Spesolimab 150 | 2 × Spesolimab 150 mg/PFS s.c. q12 |
| 300 mg Spesolimab q | mg/PFS | weeks (300 mg total dose, i.e., at |
| 12 weeks | (600 mg total/dose) | Week 12, Week 24, Week 36) |
| | | 2 × Placebo to match Solution for |
| | | Injection (Spesolimab), 1 mL PFS, at |
| | | the intervening visits (i.e., at |
| | | Week 4, Week 8, Week 16, Week 20, |
| | | Week 28, Week 32, Week 40, and |
| | | Week 44) |

Maintenance Treatment Period

| | | |
|---|---|---|
| Arm 3: | 2 × Spesolimab 150 | 1 × Spesolimab 150 mg/PFS 150 mg |
| 150 mg Spesolimab q | mg/PFS | s.c. + 1 × Placebo |
| 12 weeks | 2 × Placebo | to match Solution for Injection |
| | Spesolimab | (Spesolimab), 1 mL PFS |
| | 150 mg/ml, 1 mL | s.c. q12 weeks (i.e., at Week 12, Week |
| | PFS, (300 mg/total | 24, Week 36) |
| | dose) | 2 × Placebo to match Solution for |
| | | Injection (Spesolimab), 1 mL PFS at the |
| | | intervening visits (i.e., at |
| | | Week 4, Week 8, Week 16, Week 20, |
| | | Week 28, Week 32, Week 40, and |
| | | Week 44) |
| Arm 4: | 4 × Placebo | 2 × Placebo to match Solution for |
| Placebo | Spesolimab | Injection, (Spesolimab), 1 mL PFS s.c. |
| | 150 mg/ml, 1mL | q4 weeks |
| | PFS | |

In Case of GPP Flare:

| | |
|---|---|
| Flare Treatment | Maintenance Treatment |
| (Open Label) | (Open Label) |
| at R1/Day 1 Or at | |
| R1/Day 1 & R3/Day 8 | |
| 2 × Spesolimab 450 | 2 × Spesolimab 150 mg/PFS s.c. q12 |
| mg/vial (60 mg/mL) | weeks (300 mg total dose q12w) |
| (900 mg total dose | Or |
| i.v. infusion) | 2 × Spesolimab 150 mg/PFS s.c. q4 |
| | weeks (300 mg total dose q4w) |
| | (intensified maintenance therapy) |

### Example 3

TABLE 5
Flow Chart of Study Related Activities

| | | Randomized Treatment Period | | | | | | | | | | | | | Follow- |
|---|---|---|---|---|---|---|---|---|---|---|---|---|---|---|---|
| | Screening | Loading | Maintenance | | | | | | | | | | | | up |
| | Visit | | | | | | | | | | | | | | |
| | | | | | | | | | | | | | | V14/ | V15/ |
| | V1 | V2 | V3 | V4 | V5 | V6 | V7 | V8 | V9 | V10 | V11 | V12 | V13 | EoS1¹⁴ | EoS2¹⁵ |
| Week | −12 to 0 | 1 | 4 | 8 | 12 | 16 | 20 | 24 | 28 | 32 | 36 | 40 | 44 | 48 | 16 |
| | | | | | | | | | | | | | | | wks |
| | | | | | | | | | | | | | | | after |
| | | | | | | | | | | | | | | | last |
| | | | | | | | | | | | | | | | dose |
| Day | −84 to −1 | 1 | 29 | 57 | 85 | 113 | 141 | 169 | 197 | 225 | 253 | 281 | 309 | 337 | |
| Visit | N.A. | 0 | ±7 | ±7 | ±7 | ±7 | ±7 | ±7 | ±7 | ±7 | ±7 | ±7 | ±7 | ±7 | ±7 |
| Window | | | | | | | | | | | | | | | |
| (days) | | | | | | | | | | | | | | | |
| Whole | | X | | | | | | | | | | | | | |
| Blood for | | | | | | | | | | | | | | | |
| DNA re- | | | | | | | | | | | | | | | |
| sequencing- | | | | | | | | | | | | | | | |
| prior to | | | | | | | | | | | | | | | |
| dose | | | | | | | | | | | | | | | |
| DNA | | X | | | | | | | | | | | | | |
| banking | | | | | | | | | | | | | | | |
| sample | | | | | | | | | | | | | | | |
| (optional)⁷- | | | | | | | | | | | | | | | |
| prior to | | | | | | | | | | | | | | | |
| dose | | | | | | | | | | | | | | | |
| Whole | | | X | | | | | X | | | | | | X | |
| Blood for | | | | | | | | | | | | | | | |
| RNA | | | | | | | | | | | | | | | |
| sequencing- | | | | | | | | | | | | | | | |
| prior to | | | | | | | | | | | | | | | |
| dose | | | | | | | | | | | | | | | |
| Soluble | | X | X | | X | | | X | | | X | | | X | |
| Protein | | | | | | | | | | | | | | | |
| Biomarkers | | | | | | | | | | | | | | | |
| in Serum- | | | | | | | | | | | | | | | |
| prior to | | | | | | | | | | | | | | | |
| dose | | | | | | | | | | | | | | | |
| Serum | | X | | | | | | X | | | | | | X | |
| Biomarker | | | | | | | | | | | | | | | |
| Banking | | | | | | | | | | | | | | | |
| (optional)¹⁰- | | | | | | | | | | | | | | | |
| prior to | | | | | | | | | | | | | | | |
| dose | | | | | | | | | | | | | | | |
| GPPGA | X | X | X | X | X | X | X | X | X | X | X | X | X | X | X |
| GPPASI | | X | X | X | X | X | X | X | X | X | X | X | X | X | X |
| Target | | X | X | | | X | | X | | | | | | X | |
| Plaque | | | | | | | | | | | | | | | |
| Severity | | | | | | | | | | | | | | | |
| Score | | | | | | | | | | | | | | | |
| (TPSS)¹⁷ | | | | | | | | | | | | | | | |
| PSS; | | X | X | X | X | X | X | X | X | X | X | X | X | X | |
| PainVAS^{11a,11d} | | | | | | | | | | | | | | | |
| DLQI; EQ-5D- | | X | X | X | X | | | X | | | X | | | X | |
| 5L; PGI-S; | | | | | | | | | | | | | | | |
| PGI-C^{11a,11b,11d} | | | | | | | | | | | | | | | |
| SF-36; WPAI- | | X | | | X | | | X | | | X | | | X | |
| GPP^{11a,11c,11d} | | | | | | | | | | | | | | | |
| HCRU¹² | | X | X | X | X | X | X | X | X | X | X | X | X | X | |
| JDA GPP | | X | X | X | X | X | X | X | X | X | X | X | X | X | X |
| Severity | | | | | | | | | | | | | | | |
| Score | | | | | | | | | | | | | | | |
| IRT call | X | X | X | X | X | X | X | X | X | X | X | X | X | x²¹ | |
| Dispense/ | | X | X | X | X | X | X | X | X | X | X | X | X | | |
| Administer | | | | | | | | | | | | | | | |
| study drug | | | | | | | | | | | | | | | |
| Local | | X | X | X | X | X | X | X | X | X | X | X | X | | |
| tolerability¹³ | | | | | | | | | | | | | | | |
| Adverse | X | X | X | X | X | X | X | X | X | X | X | X | X | X | X |
| events | | | | | | | | | | | | | | | |
| Concomitant | X | X | X | X | X | X | X | X | X | X | X | X | X | X | X |
| therapy | | | | | | | | | | | | | | | |
| Study | | | | | | | | | | | | | | X | X |
| Completion | | | | | | | | | | | | | | | |
| Vital Status | | | | | | | | | | | | | | X¹⁹ | X²⁰ |
| Open Label | | | | | | | | | | | | | | X | |
| Extension | | | | | | | | | | | | | | | |
| Trial | | | | | | | | | | | | | | | |

ADA, anti-drug antibody;
C, complete physical examination;
CGI-I, Clinical Global Impression-Improvement;
d, Day;
DLQI, Dermatology Quality of Life Index;
ECG, Electrocardiogram;
EoS, end of study;
EQ-5D-5L, EuroQoL 5 Dimension 5 Level
GPPASI, Generalized Pustular Psoriasis Area and Severity Index:
GPPGA, Generalized Pustular Psoriasis Physician Global Assessment;
HCRU, Healthcare Resource Utilization;
IHC, Immunohistocompatibility Complex;
IRT, Interactive Response Technology;
JDA, Japanese Dermatological Association;
Nab, neutralizing Antibodies;
PGI-C, Patients' Global Impressions of Change;
PGI-S, Patients' Global Impressions of Severity;
PK, pharmacokinetic;
OLE, Open Label Extension,
PRO, Patient Reported Outcome;
PSS, Psoriatic Symptom Scale;
RNASeq, RNA Sequencing;
S, serum;
T, Targeted physical examination;
TPSS, Target Plaque Severity Score;
U, urine;
V, Visit;
VAS, Visual Analog Scale;
¹ a) Infection testing included tuberculosis, hepatitis B, hepatitis C, and HIV assessments. For patients who signed the informed consent ≥4 weeks prior to V2, infection testing was to have been repeated 2 to 4 weeks prior to V2.
²IL36RN mutation status was obtained from the patient's historical data if available.
³Physical examination: C = complete, T = targeted.
⁴Vital signs: a) On non-study-drug administration days, vital sign assessments were to be done prior to blood sampling; b) On study drug administration days, vital signs were assessed at predose (prior to blood sampling), 10 mins after the end of drug administration and at Visit 2 and Visit 3 additional measurements were performed approximately 60 minutes after the end of drug administration (i.e., 60 minutes after last injection).
⁵Only applicable for women of childbearing potential. S—serum pregnancy test (performed at screening). U—urine pregnancy tests were performed at all other visits indicated. Urine pregnancy testing was to have been done prior to study drug administration. Study drug was only to be administered in case of a negative test result. In case of a positive urine pregnancy test, a serum pregnancy (S) test was done (via central lab) and study drug administration was postponed until negative result of serum pregnancy test was available.
⁶ECG measurements was to always precede blood sampling and drug administration.
⁷Collection of DNA Biobanking sample was optional. Participating patients were required to give informed consent specifically for Biobanking. DNA biobanking required only one blood sample to be taken, preferably at Visit 2 (Randomization). However, collection at later visits was permitted as long as the informed consent for biobanking remained valid.
⁸Safety laboratory tests included clinical chemistry, hematology, coagulation, and urinalysis, and was performed centrally
⁹Photographs of skin/skin lesions were to precede skin biopsies and study drug administration.
¹⁰Collection of serum biobanking sample was optional. Participating patients were required to give informed consent specifically for biobanking. Samples were stored at a Biobanking facility for future research.
^{11a}These measurements were to be completed by the patient on their own, without any help from or interpretation by other people. If the patient was too sick to complete the questionnaires themselves but was able to reply verbally, a member of the study team read the instructions, questions, and response options aloud to the patient and collected the patient's verbal response in as neutral and unbiased manner as possible. If this was not possible either, the questionnaires were not to be completed. The order of completion for PROs was recommended to be as following: PSS; DLQI: PainVAS; EQ-5D-5L; WPAI-GPP; SF-36; PGI-S and PGI-C.
^{b}DLQI, EQ-5D-5L, PGI-S and PGI-C were to be completed at V2, V3, V4, V5, V8, V11 and V14/EoS1 visit only.
^{c}SF-36, WPAI-GPP was to be completed at V2, V5, V8, V11 and V14/EoS1 visit only.
^{d}For PRO completion by the adolescent patients, please refer to Section 6.2 (see CTP, Appendix 16.1.1) for further guidance and details.
¹² HCRU (healthcare resource utilization) data included the number of hospitalizations and length of stay in days, as well as the number of outpatient visits and emergency room visits (If applicable). Data should have been collected for the time period since the last visit.
¹³Local tolerability at the administration site of Spesolimab/placebo (s.c.) was assessed during the study drug administration and at the time of AE assessment by questioning retrospectively since the last visit. Any observed local tolerability reaction (s), e.g., swelling, induration, heat, redness, pain, and other findings should have been reported as an adverse event.
¹⁴V14 was recorded as the End of Study visit (i.e., EoS1) for patients who qualify and agree to enter the OLE trial (1368-0025). V14 was recorded as End of Study Visit (EoS1) for patients who prematurely discontinued with the last dose of treatment up to and including Day 232 and who agreed to complete all remaining study visits up to Wk 48 from randomization. Since these patients prematurely discontinued, they would not qualify to enter OLE trial..
¹⁵EoS2 visit was applicable for patients who did not qualify or who did not agree to enter OLE trial (1368-0025) at Week 48. EoS2 is also applicable for patients who prematurel discontinued with the last dose of treatment after Day 232. Since these patients prematurely discontinued, they did not qualify to enter OLE trial.
¹⁶Skin biopsy was optional and was only to be done for patients who have consented for this procedure. V2 and V14 skin biopsies (optional): 1 lesional or non lesional (if no lesions were present) skin biopsy of 5 mm punch (split into half for IHC and RNASeq) was to have been collected prior to study drug administration.
¹⁷Only applicable for patients with concurrent plaque psoriasis, the Target Plaque Severity Score (TPSS) was captured.
¹⁸ Only applicable if infection testing was not done at V14/EoS1 visit.
¹⁹Vital Status was collected at V14 for patients who prematurely discontinued with the last dose of treatment up to and including Day 232 and who agreed to be contacted further but did not agree for physical visits.
²⁰Vital Status was collected at V15 for patients who prematurely discontinued with the last dose of treatment after Day 232 and who agreed to be contacted further but did not agree for physical visits.
²¹IRT registration was expected once at End of Study visit (as applicable) for a patient.

### Example 4: Subject Demographics and Relevant Baseline Efficacy Parameters

The study population consisted of 38.2% men and 61.8% women. The mean age was 40.4 (range: 14 to 75) years with 8 (6.5%) adolescent subjects (2 per treatment arm); 64.2% of subjects were Asian and 35.8% were Caucasian (Table 6). Subjects included in the study had a GPPGA pustulation sub score of 1 (28.5%) or 0 (71.5%), and subjects had a GPPGA total score of 1 (86.2%) or 0 (13.8%). At the time of randomisation, 74.8% of subjects were treated with systemic therapy for GPP, which was discontinued at the start of the randomized study treatment.

Given the genetic, ethnic, and geographical heterogeneity in prevalence and severity of GPP, the efficacy of treatments in specific patient populations was investigated, e.g., a higher prevalence of interleukin-36 mutations in the Asian population has previously been linked with overall greater disease severity. Post-hoc analysis from Effisayil® 2 study showed that the effect of spesolimab in the prevention of flares was similar between the Asian and overall population.

TABLE 6
Demographics and Baseline Characteristics for Current Flare

| | | | Spesolimab | | | | | | | |
|---|---|---|---|---|---|---|---|---|---|---|
| | | | Low dose | | Medium dose | | high dose | | | |
| | | | (LD 300 mg, | | (LD 600 mg, | | (LD 600 mg, | | Overall | |
| | Placebo | | 150 mg q12w) | | 300 mg q12w) | | 300 mg q4w) | | Total | |
| Number of patients, N (%) | 31 | (100.0) | 31 | (100.0) | 31 | (100.0) | 30 | (100.0) | 123 | (100.0) |
| Gender, N (%) | | | | | | | | | | |
| Male | 13 | (41.9) | 11 | (35.5) | 11 | (35.5) | 12 | (40.0) | 47 | (38.2) |
| Female | 18 | (58.1) | 20 | (64.5) | 20 | (64.5) | 18 | (60.0) | 76 | (61.8) |
| Race, N (%) | | | | | | | | | | |
| Asian | 17 | (54.8) | 20 | (64.5) | 21 | (67.7) | 21 | (70.0) | 79 | (64.2) |
| White | 14 | (45.2) | 11 | (35.5) | 10 | (32.3) | 9 | (30.0) | 44 | (35.8) |
| Ethnicity, N (%) | | | | | | | | | | |
| Hispanic/Latino | 3 | (9.7) | 3 | (9.7) | 0 | (0.0) | 1 | (3.3) | 7 | (5.7) |
| Not Hispanic/Latino | 28 | (90.3) | 28 | (90.3) | 31 | (100.0) | 29 | (96.7) | 116 | (94.3) |
| Age [years], mean (SD) | 39.5 | (14.0) | 38.9 | (16.5) | 42.9 | (16.7) | 40.2 | (16.4) | 40.4 | (15.8) |
| Adolescents (≥12 to <18), | 2 | (6.5) | 2 | (6.5) | 2 | (6.5) | 2 | (6.7) | 8 | (6.5) |
| N (%) | | | | | | | | | | |
| Adults (≥18), N (%) | 29 | (93.5) | 29 | (93.5) | 29 | (93.5) | 28 | (93.3) | 115 | (93.5) |
| <50 | 23 | (74.2) | 23 | (74.2) | 21 | (67.7) | 22 | (73.3) | 89 | (72.4) |
| 50 to <65 | 7 | (22.6) | 6 | (19.4) | 8 | (25.8) | 5 | (16.7) | 26 | (21.1) |
| ≥65 | 1 | (3.2) | 2 | (6.5) | 2 | (6.5) | 3 | (10.0) | 8 | (6.5) |
| Weight [kg], mean (SD) | 75.73 | (23.92) | 71.07 | (23.58) | 71.52 | (23.01) | 68.66 | (22.88) | 71.77 | (23.21) |
| BMI [kg/m²], mean (SD) | 26.85 | (8.28) | 26.85 | (7.22) | 27.38 | (8.76) | 25.61 | (7.25) | 26.68 | (7.84) |
| <25, N (%) | 14 | (45.2) | 14 | (45.2) | 14 | (45.2) | 19 | (63.3) | 61 | (49.6) |
| 25 to <30, N (%) | 9 | (29.0) | 7 | (22.6) | 7 | (22.6) | 5 | (16.7) | 28 | (22.8) |
| ≥30, N (%) | 8 | (25.8) | 10 | (32.3) | 10 | (32.3) | 6 | (20.0) | 34 | (27.6) |
| Renal function based | | | | | | | | | | |
| on eGFR/CL_{CR}¹, N (%) | | | | | | | | | | |
| Normal | 24 | (77.4) | 24 | (77.4) | 17 | (54.8) | 26 | (86.7) | 91 | (74.0) |
| Mild impairment | 7 | (22.6) | 6 | (19.4) | 13 | (41.9) | 2 | (6.7) | 28 | (22.8) |
| Moderate impairment | 0 | (0.0) | 1 | (3.2) | 1 | (3.2) | 2 | (6.7) | 4 | (3.3) |
| Baseline potential | 0 | (0.0) | 0 | (0.0) | 0 | (0.0) | 0 | (0.0) | 0 | (0.0) |
| hepatic impairment², N (%) | | | | | | | | | | |
| Concomitant use of | 22 | (71.0) | 25 | (80.6) | 23 | (74.2) | 22 | (73.3) | 92 | (74.8) |
| systemic GPP medication | | | | | | | | | | |
| at randomization, N (%) | | | | | | | | | | |
| Concurrent plaque | 10 | (32.3) | 10 | (32.3) | 7 | (22.6) | 7 | (23.3) | 34 | (27.6) |
| psoriasis³, N (%) | | | | | | | | | | |
| Concurrent chronic | 0 | (0.0) | 3 | (9.7) | 0 | (0.0) | 0 | (0.0) | 3 | (2.4) |
| erythrodermic psoriasis, | | | | | | | | | | |
| N (%) | | | | | | | | | | |

Results for categories “not stated”, “unknown”, or “missing” are provided in the source table.
BMI: body mass index; ESRD: end stage renal disease.
¹Classification of renal function based on estimated CLCR calculated applying CKD-EPI formula: normal renal function; (stage 1) ≥90 mL/min/1.73 m2, mild decrease in GFR; (stage 2) = 60 to 89 mL/min/1.73 m2, moderate decrease in GFR; (stage 3) = 30 to 59 mL/min/1.73 m2, severe decrease in GFR (stage 4) = 15 to 29 mL/min/1.73 m2, end stage renal disease (stage 5) = <15 mL/min/1.73 m2 not on dialysis or requiring dialysis. The Bedside Schwartz Equation was applied for adolescents.
²Defined as International Normalized Ratio ≥2.2 and total serum bilirubin >51.3 μmol/L; see TSAP
³Concurrent plaque psoriasis meant that the subject had plaque psoriasis during the previous year and the condition was still ongoing at baseline.

As required by the inclusion criterion, all subjects had a GPPGA total score of 0 (clear; 13.8%) or 1 (almost clear; 86.2%) at baseline. All subjects had a GPPGA pustulation subscore of 0 (71.5%) or 1 (28.5%). At baseline, the mean (SD) was 4.2 (3.4) for PSS total score, 8.1 (6.4) for DLQI total score, and 3.29 (3.74) for GPPASI total score. Baseline efficacy variables were generally comparable across the treatment groups. However, there was some indication of a higher disease burden in subjects in the spesolimab high dose group compared with the placebo group. Mean (SD) PSS total scores were 5.3 (3.8) in the spesolimab high dose group and 3.6 (2.9) in the placebo group; DLQI total scores were 11.1 (6.9) in the spesolimab high dose group and 7.2 (5.6) in the placebo group; GPPASI total scores were 3.92 (4.42) in the spesolimab high dose group and 3.11 (2.81) in the placebo group.

TABLE 7
GPPGA, GPPASI, PS and DLQI Scores and JDA GPP severity index at baseline.

| | | | Spesolimab | | | | | | | |
|---|---|---|---|---|---|---|---|---|---|---|
| | | | low dose | | medium dose | | high dose | | | |
| | | | (LD 300 mg, | | (LD 600 mg, | | (LD 600 mg, | | Overall | |
| | Placebo | | 150 mg q12w) | | 300 mg q12w) | | 300 mg q4w) | | Total | |
| Number of patients, N (%) | 31 | (100.0) | 31 | (100.0) | 31 | (100.0) | 31 | (100.0) | 123 | (100.0) |
| GPPASI total score, mean (SD) | 3.11 | (2.81) | 3.03 | (3.48) | 3.12 | (4.16) | 3.92 | (4.42) | 3.29 | (3.74) |
| GPPGA total score, N (%) | | | | | | | | | | |
| 0 | 4 | (12.9) | 2 | (6.5) | 8 | (25.8) | 3 | (10.0) | 17 | (13.8) |
| 1 | 27 | (87.1) | 29 | (93.5) | 23 | (74.2) | 27 | (90.0) | 106 | (86.2) |
| GPPGA pustules, N (%) | | | | | | | | | | |
| 0 | 21 | (67.7) | 23 | (74.2) | 24 | (77.4) | 20 | (66.7) | 88 | (71.5) |
| 1 | 10 | (32.3) | 8 | (25.8) | 7 | (22.6) | 10 | (33.3) | 35 | (28.5) |
| JDA GPP severity index, mean (SD) | 2.1 | (1.3) | 2.0 | (1.1) | 2.0 | (1.4) | 2.2 | (1.6) | 2.1 | (1.3) |
| Mild, N (%) | 29 | (93.5) | 31 | (100.0) | 31 | (100.0) | 28 | (93.3) | 119 | (96.7) |
| Moderate, N (%) | 0 | (0.0) | 0 | (0.0) | 0 | (0.0) | 1 | (3.3) | 1 | (0.8) |
| Severe, N (%) | 0 | (0.0) | 0 | (0.0) | 0 | (0.0) | 0 | (0.0) | 0 | (0.0) |
| Missing, N (%) | 2 | (6.5) | 0 | (0.0) | 0 | (0.0) | 1 | (3.3) | 3 | (2.4) |
| PSS total score, N | 31 | | 31 | | 31 | | 29 | | 122 | |
| Mean (SD) | 3.6 | (2.9) | 4.1 | (3.8) | 3.9 | (2.9) | 5.3 | (3.8) | 4.2 | (3.4) |
| DLQI total score, N | 31 | | 30 | | 31 | | 29 | | 121 | |
| Mean (SD) | 7.2 | (5.6) | 7.6 | (6.7) | 6.6 | (5.6) | 11.1 | (6.9) | 8.1 | (6.4) |

Patients below the age of 16 years (i.e., 12 to 15 years) did not have to complete the DLQI questionnaire

In this trial, DNA sequencing of the IL-36RN, CARD14, and AP1S3 genes was performed. Presence of potential pathogenic IL-36RN variation (with amino acid substitution) was reported for 22.8% of subjects overall and was lower in the placebo group (12.9%) than in the spesolimab groups (low 22.6%, medium 32.3%, high 23.3%). Otherwise, genetic mutations were generally comparable between the treatment groups.

Most subjects received their first GPP diagnosis longer than 5 years prior to randomization. Clinical examination was the most common method for the diagnosis, followed by skin biopsy and histopathological confirmation. The median (Q1, Q3) number of flares per year was 2.0 (1.0, 3.0), with a maximum of 12. These data were generally comparable between the treatment groups (Table 8 below).

TABLE 8
Medical History for GPP

| | | | Spesolimab | | | | | | | |
|---|---|---|---|---|---|---|---|---|---|---|
| | | | low dose | | medium dose | | high dose | | | |
| | | | (LD 300 mg, | | mg, 300 mg | | (LD 600 mg, | | Overall | |
| | Placebo | | 150 mg q12w) | | (LD 600 q12w) | | 300 mg q4w) | | Total | |
| Number of patients, N (%) | 31 | (100.0) | 31 | (100.0) | 31 | (100.0) | 30 | (100.0) | 123 | (100.0) |
| Time since first diagnosis, N (%) | | | | | | | | | | |
| ≤1 year | 3 | (9.7) | 5 | (16.1) | 4 | (12.9) | 4 | (13.3) | 16 | (13.0) |
| >1 to ≤5 years | 10 | (32.3) | 6 | (19.4) | 9 | (29.0) | 9 | (30.0) | 34 | (27.6) |
| >5 to ≤10 years | 7 | (22.6) | 6 | (19.4) | 8 | (25.8) | 8 | (26.7) | 29 | (23.6) |
| >10 years | 11 | (35.5) | 14 | (45.2) | 10 | (32.3) | 9 | (30.0) | 44 | (35.8) |
| Diagnosis method to confirm GPP, N (%) | | | | | | | | | | |
| Skin biopsy and histopathological | 15 | (48.4) | 11 | (35.5) | 12 | (38.7) | 9 | (30.0) | 47 | (38.2) |
| confirmation | | | | | | | | | | |
| Clinical examination | 15 | (48.4) | 18 | (58.1) | 17 | (54.8) | 21 | (70.0) | 71 | (57.7) |
| Other | 1 | (3.2) | 2 | (6.5) | 2 | (6.5) | 0 | (0.0) | 5 | (4.1) |
| Average number of flares per year | | | | | | | | | | |
| Mean (SD) | 2.4 | (1.2) | 2.7 | (2.3) | 1.9 | (0.9) | 2.4 | (1.9) | 2.3 | (1.6) |

| | | | | | |
|---|---|---|---|---|---|
| Median (Q1, Q3) | 2.0 | 2.0 | 2.0 | 2.0 | 2.0 |
| | (2.0, 3.0) | (1.0, 4.0) | (1.0, 2.0) | (2.0, 2.0) | (1.0, 3.0) |
| Min, max | 1, 5 | 1, 12 | 0, 4 | 0, 10 | 0, 12 |

Present or past occurrence of psoriasis was reported for 80 subjects (65.0%) and arthritis for 10 subjects (8.1%); ongoing chronic plaque psoriasis was reported for 34 subjects (27.6%). Overall, 74.0% of all subjects had at least 1 baseline condition/medical history. The most common dictionary-derived terms were hypertension, psoriasis, and obesity.

Overall, 93.5% of subjects had at least 1 historical medication for GPP (i.e., medications for GPP that had been stopped prior to screening). The most frequently used historical medications at preferred name level were acitretin, methotrexate, and ciclosporin. The most common systemic medications for GPP at randomization were acitretin, ciclosporin, and methotrexate. These medications were used within 4 weeks prior to or at randomization and were discontinued before the start of the randomized study treatment.

All 123 subjects randomized in this trial received at least 1 dose of study drug. The mean (SD) duration of exposure during the randomized maintenance treatment period was higher for the spesolimab dose groups (low dose: 33.9 [18.2] weeks, medium dose: 31.9 [18.5] weeks, high dose: 33.1 [18.2] weeks) than for the placebo group (25.4 [20.8] weeks). A total of 32 subjects received spesolimab i.v. for the treatment of a flare; of these subjects, 22 subjects (68.8%) received a single 900 mg i.v. dose and 10 subjects (31.3%) received 2 doses of spesolimab 900 mg i.v. (double dose). Of the 20 subjects who continued with open label (OL) maintenance treatment with spesolimab s.c. after flare treatment, 11 subjects (55.0%) stayed on the 300 mg s.c. q12w dosing regimen and 9 subjects (45.0%) were escalated to the 300 mg s.c. q4w dosing regimen. The mean (SD) duration of exposure over the entire study was 39.3 (11.2) weeks for subjects initially randomized to placebo, 37.1 (15.0) weeks for subjects initially randomized to spesolimab low dose, 38.3 (12.9) weeks for subjects initially randomized to spesolimab medium dose, and 34.6 (17.1) weeks for subjects initially randomized to spesolimab high dose.

### Example 5: Efficacy: Primary and Key Secondary Objectives

### Summary

For the primary objective, a non-flat dose-response relationship was demonstrated. Subsequently, the confirmatory testing of the secondary objective was carried out. Spesolimab high dose demonstrated statistically significant efficacy compared with placebo based on the primary endpoint and the key secondary endpoint.

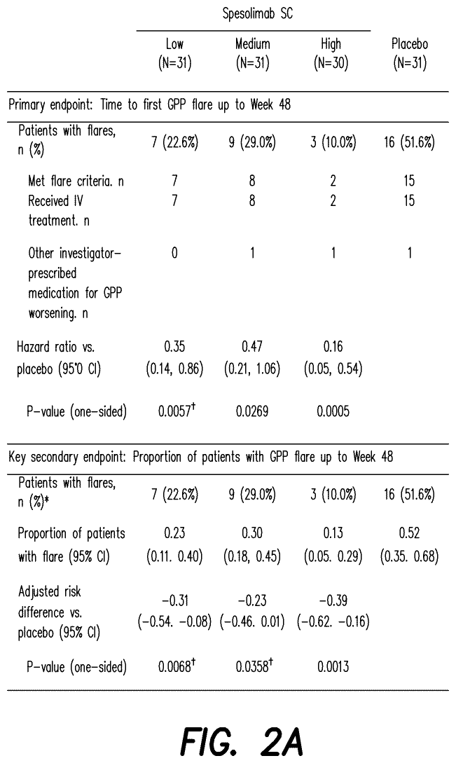

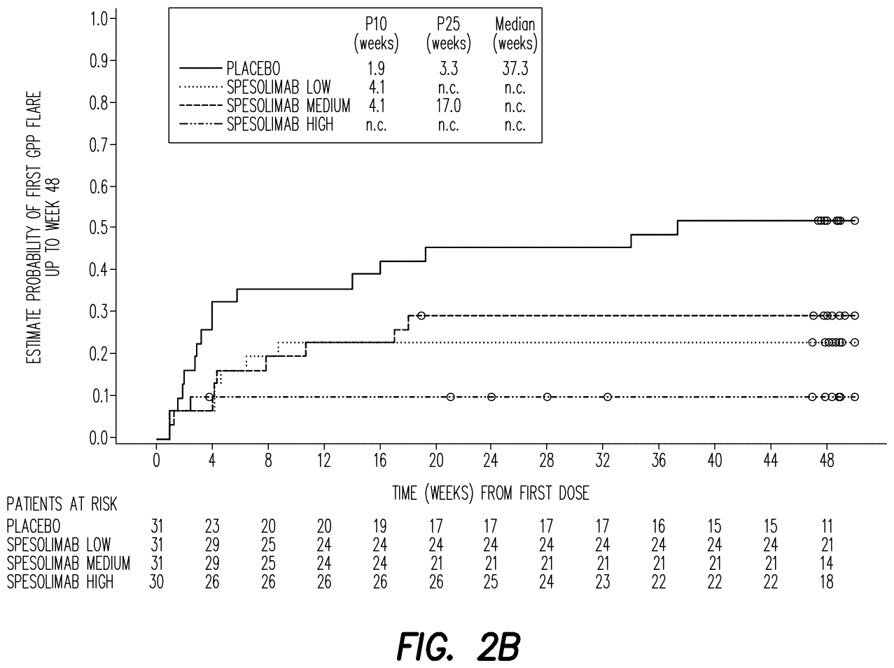

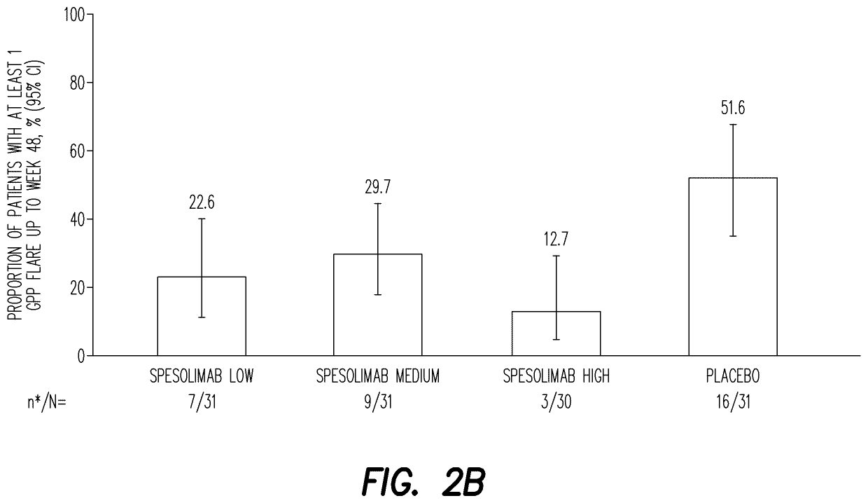

Primary Objective: By Week 48 of the trial, a total of 35 patients had GPP flares; fewer patients in the low- (7, 22.6%), medium- (9, 29.0%) and high-dose (3, 10.0%) spesolimab arms experienced GPP flares compared with the placebo arm (16, 51-6%; FIG. 2A). The non-flat dose-response relationship for spesolimab compared with placebo was established, with statistically significant P-values for each predefined model (P=0.002 for the linear, emax1, and emax2 models; P=0-003 for the exponential model); therefore, the primary trial objective was met.

TABLE 9
Time to first GPP flare and occurrence of GPP flare up to 48 Weeks

| | | | Spesolimab | | | | |
|---|---|---|---|---|---|---|---|
| | | | low dose | medium dose | | high dose | |
| | | | (LD 300 mg, | (LD 600 mg, | | (LD 600 mg, | |
| | Placebo | | 150 mg q12w) | 300 mg q12w) | | 300 mg q4w) | |
| Number of patients, N (%) | 31 | (100.0) | 31 (100.0) | 31 | (100.0) | 30 | (100.0) |
| Patients with GPP flares, N (%) | 16 | (51.6) | 7 (22.6) | 9 | (29.0) | 3 | (10.0) |
| Meeting GPP flare criteria | 15 | (48.4) | 7 (22.6) | 8 | (25.8) | 2 | (6.7) |
| Received spesolimab i.v. | 15 | (48.4) | 7 (22.6) | 8 | (25.8) | 2 | (6.7) |
| Spesolimab i.v. treatment | 0 | | 0 | 0 | | 0 | |
| as a rescue medication | | | | | | | |
| (without meeting GPP | | | | | | | |
| flare criteria) | | | | | | | |
| Investigator-prescribed | 1 | (3.2) | 0 | 1 | (3.2) | 1 | (3.3) |

| SoC to treat GPP | | | | |
|---|---|---|---|---|
| worsening without | | | | |
| meeting the GPP flare | | | | |
| criteria | | | | |
| Event time [week], 10^{th} | 1.9 (0.9, | 4.1 (1.0, | 4.1 (0.9, | n.c. (1.0, |
| percentile (95% CI) ¹ | 2.9) | 8.7) | 10.7) | n.c.) |
| Probability of event at | 0.516 (0.356, | 0.226 (0.115, | 0.290 (0.163, | 0.100 (0.033, |
| Week 48 (Day 337), | 0.698) | 0.416) | 0.484) | 0.279) |
| Kaplan-Meier (KM) | | | | |
| estimate (95% CI) | | | | |
| HR for the time to the first | | 0.350 (0.143, | 0.468 (0.206, | 0.157 0.046, |
| flare vs placebo (95% CI) ² | | 0.857) | 1.064) | 0.541) |
| p-value ³ | | 0.0057 | 0.0269 | 0.0005 |

(nominal)

GPP flare criteria: increase in GPPGA score by ≥2 from baseline and GPPGA pustulation subscore ≥2 up to Week 48. The use of spesolimab i.v. treatment as a rescue medication or investigator-prescribed standard of care (SoC) to treat GPP worsening were considered as onset of GPP flare.
n.c., not calculable
¹ KM estimate of the time when 10% of the patients had endpoint events
³ Cox regression model stratified by the use of systemic GPP medications at randomization
³ Log-rank test stratified by the use of systemic GPP medications at randomization

Secondary Objective: A lower number of subjects in all spesolimab groups than in the placebo group had GPP flares up to Week 48. The low-, medium-, and high-dose regimens of spesolimab numerically reduced the risk of a GPP flare over 48 weeks, with hazard ratios of 0-35 (95% CI, 0.14 to 0-86; nominal P=0.0057), 0.47 (95% CI, 0.21 to 1-06; P=0.027), and 0.16 (95% CI, 0-05 to 0-54; P=0.0005), respectively (FIG. 2A). Spesolimab high dose was statistically significant in reducing the risk of GPP flares compared with placebo, with an HR of 0.157 (95% CI 0.046, 0.541; p=0.0005).

The separation of the estimated probability of the first GPP flare between spesolimab groups and placebo started in the first 4 weeks after randomization and was maintained up to Week 48 (FIG. 2B). After 4 weeks, no flare was reported in the high dose group. Analysis of the key secondary endpoint revealed risk differences for the occurrence of a GPP flare vs. placebo over 48 weeks of −0-31 (95% CI, −0-54 to −0-08; nominal P=0-0068), −0-23 (95% CI, −0-46 to 0-01; nominal P=0-036) and −0-39 (95% CI, −0-62 to −0-16; P=0-0013) for the low, medium, and high doses of spesolimab, respectively (FIGS. 2A and C). Using a one-sided alpha of 0.00625 (adjusted for multiplicity), the high-dose spesolimab regimen achieved statistically significant improvement over placebo on the key secondary endpoint (P=0.0013).

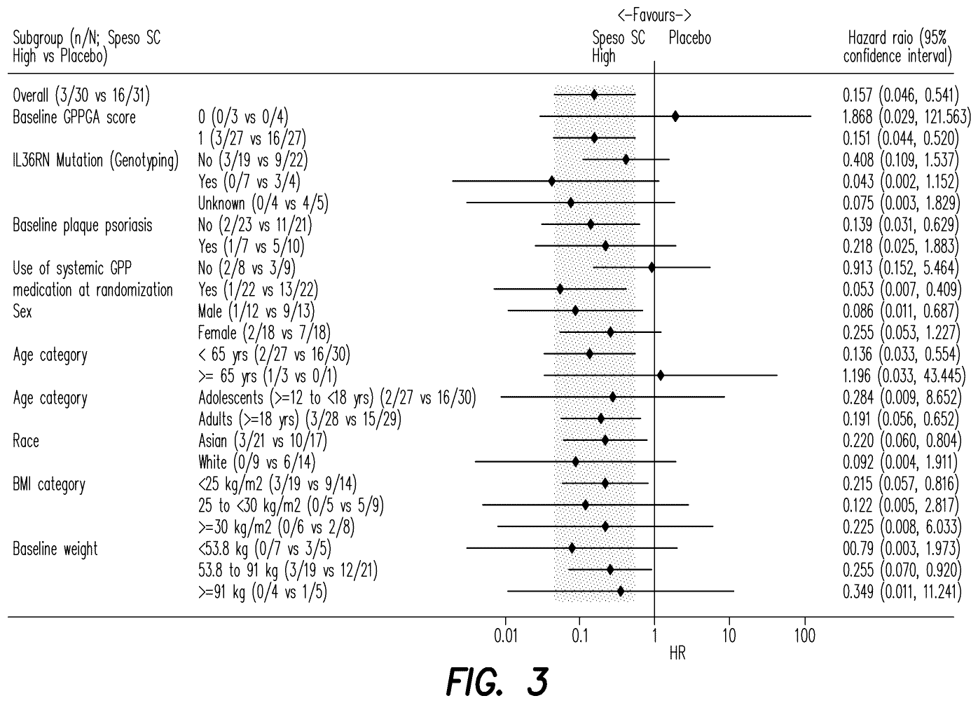

The primary analysis of spesolimab high dose vs placebo for the time to the first GPP flare up to Week 48 was generally consistent across the subgroups (See FIG. 3)

### Example 6: Primary Efficacy Outcomes

The study endpoints were selected with the aim of establishing the efficacy and safety of spesolimab for the prevention of GPP flares, with maximal statistical power. Systemic aspects of the GPP flares were assessed using measures including components of the Japanese Dermatological Association GPP severity score, which was developed for the Japanese Ministry of Health as a diagnostic tool for measuring the severity of GPP at presentation (Fujita H, Terui T, Hayama K, Akiyama M, Ikeda S, Mabuchi T, et al. Japanese guidelines for the management and treatment of generalized pustular psoriasis: the new pathogenesis and treatment of GPP. J Dermatol. 2018; 45(11):1235-70). A range of subject (patient)-reported outcomes (PROs) are measured providing unique insights into the impact of GPP and the trial intervention from the subjects' perspective, and PROs are considered an important factor by dermatologists when making treatment decisions in subjects with psoriasis (Mercieca-Bebber R, King M T, Calvert M J, Stockler M R, Friedlander M. The importance of patient-reported outcomes in clinical trials and strategies for future optimization. Patient Relat Outcome Meas. 2018; 9:353-67; . Feldman S R, Regnier S A, Chirilov A, Hey F, Gilloteau I, Cella D). Some of these PROs (Psoriasis Symptom Scale, Pain Visual Analogue Scale, Dermatology Life Quality Index) were used successfully in the Effisayil™ 1 study (Bachelez H, Choon S E, Marrakchi S, Burden A D, Tsai T F, Morita A, et al. Trial of spesolimab for generalized pustular psoriasis. N Engl J Med. 2021; 385(26):2431-40) and were also used in the Effisayil™ 2 study to evaluate participants' health-related quality of life, ability to participate in daily activities, and experience of pain.

TABLE 10
Overview of efficacy endpoints in the randomized
maintenance period up to Week 48

| Efficacy endpoint in the | | Frequency of | | |
|---|---|---|---|---|
| randomized maintenance | Assessed | scheduled assessment | Endpoint | In testing |
| period up to Week 48 | by | up to Week 48 | type | strategy |
| Time to event | | | | |
| Time to the first GPP flare | Clinician | GPPGA every 4 | Primary | Yes |
| | | weeks | | |
| Time to the first worsening | Patient | PSS every 4 weeks | Secondary | Yes |
| of PSS | | | | |
| Time to the first worsening | Patient | DLQI Week 4, 8, 12, | Secondary | Yes |
| of DLQI | | 24, 36, 48 | | |
| Binary | | | | |
| Occurrence of at least 1 | Clinician | GPPGA every 4 | Key | Yes |
| GPP flare | | weeks | secondary | |
| Sustained remission | Clinician | GPPGA every 4 | Secondary | No |
| | | weeks | | |
| Modified sustained | Clinician | GPPGA every 4 | Further | No |
| remission | | weeks | | |
| No PSS sub-score >1 | Patient | PSS every 4 weeks | Further | No |
| at ≥75% of visits | | | | |
| DLQI of 0 or 1 at all visits | Patient | DLQI Week 4, 8, 12, | Further | No |
| | | 24, 36, 48 | | |
| Continuous | | | | |
| WPAI score over time | Patient | Every 12 weeks | Further | No |
| GPPGA score over time | Clinician | Every 4 weeks | Further | No |
| GPPASI score over time | Clinician | Every 4 weeks | Further | No |
| SF-36 score over time | Patient | Every 12 weeks | Further | No |
| Pain VAS score over time | Patient | Every 4 weeks | Further | No |
| EQ-5D-5L score over time | Patient | Week 4, 8, 12, 24, | Further | No |
| | | 36,48 | | |
| JDA GPP severity score | Clinician | Every 4 weeks | Further | No |
| over time | | | | |
| TPSS, over time | Clinician | Week 4, 16, 24, 48 | Further | No |
| PGI-S, over time | Patient | Week 4, 8, 12, 24, | Further | No |
| | | 36, 48 | | |
| PGI-C, over time | Patient | Week 4, 8, 12, 24, | Further | No |
| | | 36,48 | | |

Patients were to make unscheduled clinical visits if GPP flares were suspected in between the protocol-specified scheduled visits

### Primary Efficacy Analysis:

Efficacy of treatment was measured as the time to first Generalized Pustular Psoriasis (GPP) flare (defined by increase in Generalized Pustular Psoriasis Physician Global Assessment (GPPGA) score by ≥2 from baseline and the pustular component of GPPGA≥2) up to Week 48.

Up to Week 48, a total of 35 patients had GPP flares, with 32 patients meeting the GPP flare criteria, who all received OL spesolimab i.v. treatment, and 3 patients not meeting the GPP flare criteria but receiving investigator prescribed SoC to treat GPP worsening. A lower number of patients in the spesolimab low (7 patients), medium (9 patients), and high dose (3 patients) groups than in the placebo group (16 patients) had flares.

For the primary objective, the adjusted p-value for each pre-defined model (0.002 for linear, emax1, and emax2; 0.003 for exponential) was statistically significant, thus demonstrating a non-flat dose-response relationship for spesolimab compared with placebo for the primary endpoint.

For the secondary objective, spesolimab high dose was statistically significant in reducing the risk of GPP flares compared with placebo in the primary analysis, with an HR of 0.157 (95% CI 0.046, 0.541; p=0.0005). The HR was 0.350 (95% CI 0.143, 0.857; nominal p=0.0057) for the low dose and 0.468 (95% CI 0.206, 1.064; p=0.0269) for the medium dose. The medium dose did not reach statistical significance and the key secondary endpoint was only further evaluated for the high dose. The separation of the estimated probability of the first GPP flare between spesolimab groups and placebo started in the first 4 weeks after randomization and was maintained up to Week 48. After 4 weeks, no flare was reported in the high dose group (see FIG. 2).

Sensitivity analyses using alternative censoring method or patient analysis set under the primary estimand, and additional analyses under different estimands were consistent with the primary analysis. The primary analysis of spesolimab high dose vs placebo for the time to the first GPP flare up to Week 48 was generally consistent across the subgroups.

TABLE 11
Time to the first GPP flare and occurrence of GPP flare up to Week 48

| | | | Spesolimab | | | | |
|---|---|---|---|---|---|---|---|
| | | | low dose | medium dose | | high dose | |
| | | | (LD 300 mg, | (LD 600 mg, | | (LD 600 mg, | |
| | Placebo | | 150 mg q12w) | 300 mg q12w) | | 300 mg q4w) | |
| Number of patients, N (%) | 31 | (100.0) | 31 (100.0) | 31 | (100.0) | 30 | (100.0) |
| Patients with GPP flares, N (%) | 16 | (51.6) | 7 (22.6) | 9 | (29.0) | 3 | (10.0) |
| Meeting GPP flare criteria | 15 | (48.4) | 7 (22.6) | 8 | (25.8) | 2 | (6.7) |
| Received spesolimab i.v. | 15 | (48.4) | 7 (22.6) | 8 | (25.8) | 2 | (6.7) |
| Spesolimab i.v. treatment | 0 | | 0 | 0 | | 0 | |
| as a rescue medication | | | | | | | |
| (without meeting GPP flare | | | | | | | |
| criteria | | | | | | | |
| Investigator-prescribed | 1 | (3.2) | 0 | 1 | (3.2) | 1 | (3.3) |

| SoC to treat GPP | | | | |
|---|---|---|---|---|
| worsening without meeting | | | | |
| the GPP flare criteria | | | | |
| Event time [week], 10^{th} | 1.9 (0.9, | 4.1 (1.0, | 4.1 (0.9, | n.c. (1.0, |
| percentile (95% CI) ¹ | 2.9) | 8.7) | 10.7) | n.c.) |
| Probability of event at Week | 0.516 (0.356, | 0.226 (0.115, | 0.290 (0.163, | 0.100 (0.033, |
| 48 (Day 337), Kaplan-Meier | 0.698) | 0.416) | 0.484) | 0.279) |
| (KM) estimate (95% CI) | | | | |
| HR for the time to the first | | 0.350 (0.143, | 0.468 (0.206, | 0.157 (0.046, |
| flare vs placebo (95% CI) ² | | 0.857) | 1.064) | 0.541) |
| p-value ³ | | 0.0057 | 0.0269 | 0.0005 |

(nominal)

GPP flare criteria: increase in GPPGA score by ≥2 from baseline and GPPGA pustulation subscore ≥2 up to Week 48. The use of spesolimab i.v. treatment as a GPP flare medication or investigator-prescribed standard of care (SoC) to treat GPP worsening were considered as onset of GPP flare.
n.c., not calculable
¹ KM estimate of the time when 10% of the subjects had endpoint events
² Cox regression model stratified by the use of systemic GPP medications at randomization
³ Log-rank test stratified by the use of systemic GPP medications at randomization

### Secondary Efficacy Endpoints:

### A. The Occurrence of at Least One GPP Flare Up to Week 48:

The proportion of patients with at least 1 GPP flare (defined by increase in GPPGA score by 2 from baseline and the pustular component of GPPGA 2) up to Week 48 was lower in all spesolimab groups than in the placebo group. Spesolimab high dose was statistically significant in reducing the occurrence of GPP flares compared with placebo, with an adjusted risk difference of −0.390 (95% CI −0.621, −0.159; p=0.0013). The difference between spesolimab medium dose and placebo did not reach statistical significance (−0.225; 95% CI −0.462, 0.013; nominal p=0.0358). The occurrence of GPP flares was lower with spesolimab low dose compared with placebo (−0.308; 95% CI −0.535, −0.081; nominal p=0.0068).

TABLE 12
The proportion of patients with at least 1 GPP flare up to Week 48

| | | Spesolimab | | |
|---|---|---|---|---|
| | | low dose | medium dose | high dose |
| | | (LD 300 mg, | (LD 600 mg, | (LD 600 mg, |
| | Placebo | 150 mg q12w), | 300 mg q12w) | 300 mg q4w) |
| Number of patients, N | 31 | 31 | 31 | 30 |
| Proportion with GPP flares | 0.516 (0.348, | 0.226 (0.114, | 0.297 (0.181, | 0.127 (0.050, |
| (95% CI) | 0.680) | 0.398) | 0.445) | 0.289) |
| Risk difference for GPP flare | | (−0.535, −0.081) | −0.225 | −0.390 |
| occurrence vs placebo (95% | | | (−0.462, 0.013) | (−0.621, −0.159) |
| CI) ¹ | | | | |
| p-value | | 0.0068 | 0.0358 | 0.0013 |
| | | (nominal) | (nominal) | |

The use of spesolimab i.v. treatment as a rescue medication or investigator- prescribed SoC to treat GPP worsening were considered as onset of GPP flare
¹ Cochran-Mantel-Haenszel test after multiple imputation, stratified by the use of systemic GPP medications at randomization.

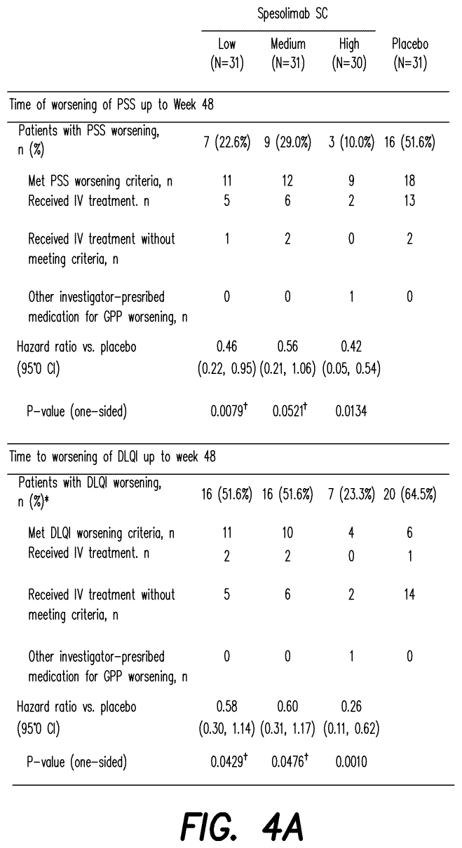

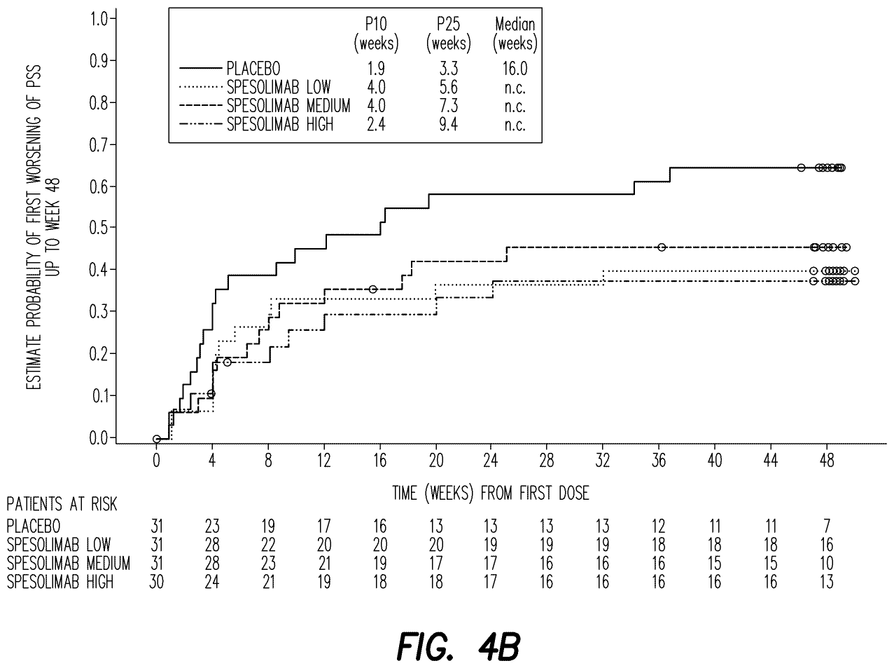

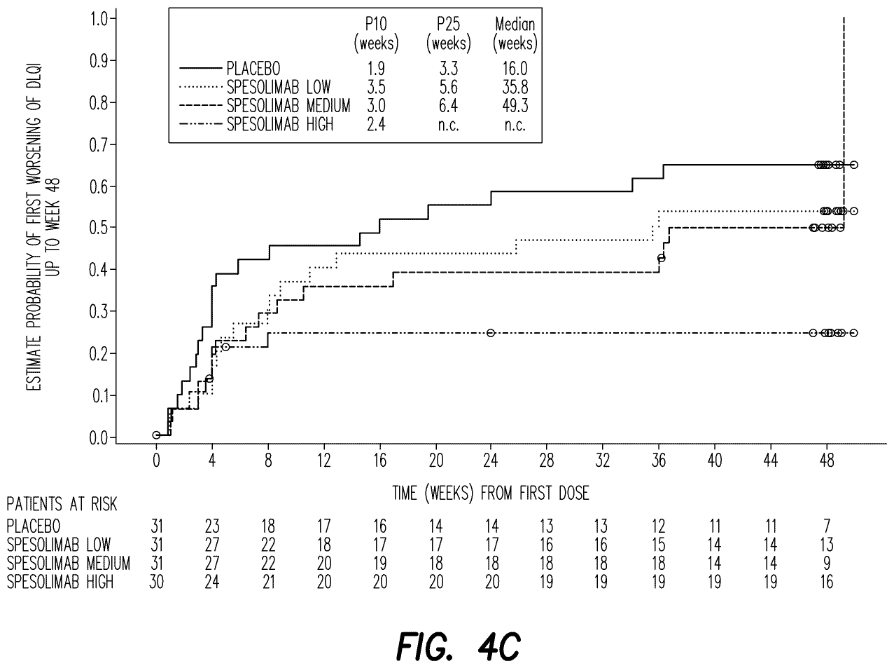

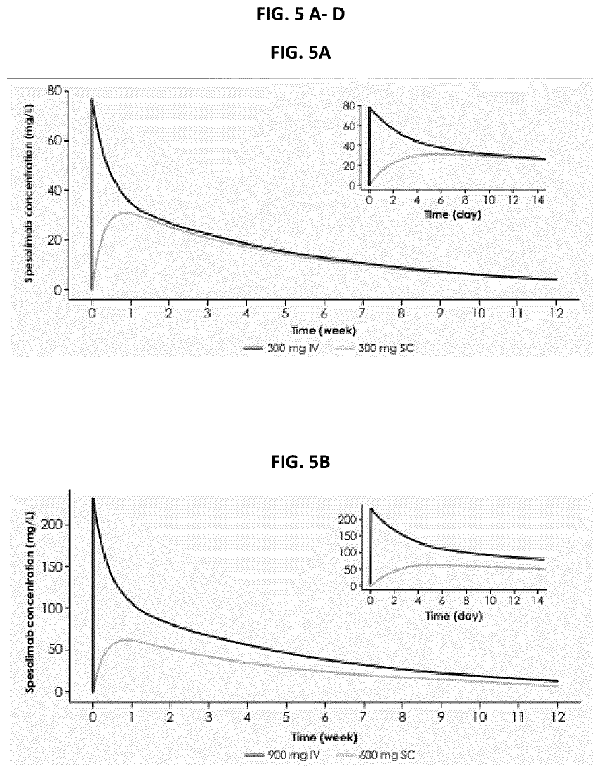

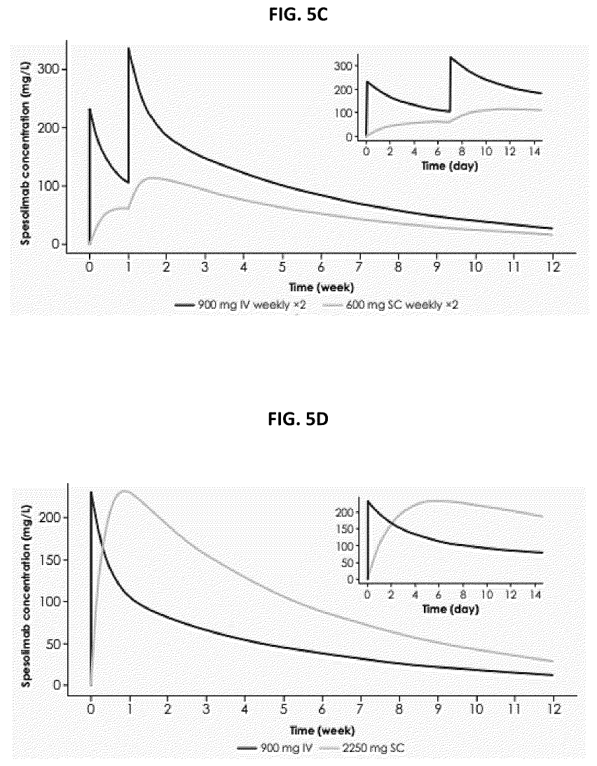

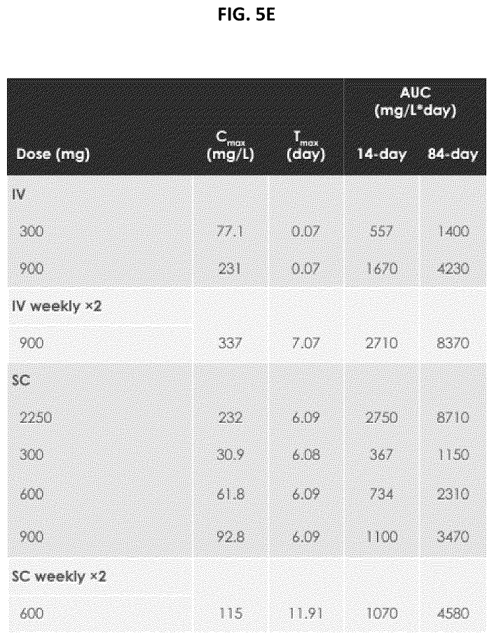

Landmark analysis illustrated a lower proportion of patients with flares in the spesolimab high dose group than placebo, which was evident at Week 12 and continued through Week 48 (FIG. 4). The primary analysis of spesolimab high dose vs placebo for the occurrence of at least 1 GPP flare up to Week 48 was generally consistent across the subgroups (FIG. 5). The subgroup results with risk difference point estimates not within the 95% CI of the overall analysis were based on small numbers of patients and very few endpoint events (FIG. 5)

### B. Time to First Worsening of Psoriasis Symptom Scale (PSS) Up to Week 48:

Time to first worsening of Psoriasis Symptom Scale (PSS) up to Week 48 defined as a 4-point increase in total score from baseline. Intake of rescue medication, or investigator prescribed SoC, was considered as onset of a worsening.

Spesolimab reduced the risk of PSS worsening over 48 weeks compared with placebo, as demonstrated by hazard ratios of 0.46 (95% CI, 0.22 to 0-95; nominal P=0.0079), 0-56 (95% CI, 0.28 to 1.10; nominal P=0.052), and 0.42 (95% CI, 0.20 to 0.91; P=0.013) for the low-, medium-, and high-dose regimens, respectively (FIG. 4A). A smaller proportion of patients in the low- (12/31, 38.7%), medium- (14/31, 45.2%), and high-dose (10/30, 33.3%) spesolimab arms reported a worsening of their PSS score compared with the placebo arm (20/31, 64.5%) (FIG. 4B). Since the required significance level of 0.00625 was not reached for the high dose, confirmatory testing stopped. However, the separation of the estimated probability of PSS worsening between spesolimab groups and placebo started in the first 8 weeks after randomization and was maintained up to Week 48. Data from the Effisayil™ ON study, the open label extension for patients from the Effaisayil™1 study who either received one or two doses of 900 mg spesolimab i.v. support the need for q4w dosing of 300 mg as compared to q12w dosing when initiating treatment for prevention of GPP flares. One-third (33.3% [36/108]) of participants who started q12w dosing in Effisayil® ON were either escalated to a q4w regimen (n=24) or experienced a flare (n=12).

Taken together, the Effisayil® 2 and Effisayil® ON results suggest that spesolimab LD 600 mg/SC 300 mg q4w is the optimal dosing regimen for prevention of GPP flares

TABLE 13
Time to the first worsening of PSS up to Week 48:

| | | | Spesolimab | | | | | |
|---|---|---|---|---|---|---|---|---|
| | | | low dose | | medium dose | | high dose | |
| | | | (LD 300 mg, | | (LD 600 mg, | | (LD 600 mg, | |
| | Placebo | | 150 mg q12w) | | 300 mg q12w) | | 300 mg q4w) | |
| Number of patients, N (%) | 31 | (100.0) | 31 | (100.0) | 31 | (100.0) | 30 | (100.0) |
| Patients with PSS worsening, N (%) | 20 | (64.5) | 12 | (38.7) | 14 | (45.2) | 10 | (33.3) |
| Meeting PSS worsening criterion | 18 | (58.1) | 11 | (35.5) | 12 | (38.7) | 9 | (30.0) |
| Received spesolimab i.v. | 13 | (41.9) | 5 | (16.1) | 6 | (19.4) | 2 | (6.7) |

| | | | | | | | |
|---|---|---|---|---|---|---|---|
| Spesolimab i.v. treatment as a | 2 | (6.5) | 1 | (3.2) | 2 | (6.5) | 0 |

| rescue medication (without | | | | | |
|---|---|---|---|---|---|
| meeting PSS worsening criterion | | | | | |
| Investigator-prescribed SoC to | 0 | 0 | 0 | 1 | (3.3) |

| | | | | |
|---|---|---|---|---|
| treat GPP worsening | | | | |
| Event time [week], 25^{th} | 3.3 (1.6, | 5.6 (4.0, | 7.3 (3.0, | 9.4 (1.1, |
| percentile (95% CI) ¹ | 8.6) | 32.0) | 18.1) | n.c.) |
| Probability of event at Week 48 | 0.645 (0.481, | 0.400 (0.250, | 0.457 (0.301, | 0.376 (0.223, |
| (Day 337), KM estimate (95% CI) | 0.806) | 0.595) | 0.647) | 0.588) |
| HR for the time to the first | | 0.459 (0.222, | 0.555 (0.278, | 0.424 (0.197, |
| worsening vs placebo (95% CI) ² | | 0.945) | 1.104) | 0.914) |
| p-value ³ | | 0.0079 | 0.0521 | 0.0134 |

| | |
|---|---|
| (nominal) | (nominal) |

PSS worsening criterion: 4-point increase in total score from baseline up to Week 48. The use of spesolimab i.v. treatment as a GPP flare treatment medication or investigator prescribed SoC to treat GPP worsening were considered as onset of PSS worsening.
Patients with baseline values missing (1 patient in spesolimab high dose group, or higher than 12 (maximum score possible is 16; 1 patient each in spesolimab low dose and high dose groups, were censored at Day 1
¹ KM estimate of the time when 25% of the patients had endpoint events
² Cox regression model stratified by the use of systemic GPP medications at randomization.
³ Log-rank test stratified by the use of systemic GPP medications at randomization.

### C. Time to First Worsening of Dermatology Quality of Life Index (DLQI) Up to Week 48:

Time to first worsening of Dermatology Quality of Life Index (DLQI) up to Week 48 defined as a 4-point increase in total score from baseline. Intake of rescue medication, or investigator prescribed SoC, was considered as onset of a worsening.

Up to Week 48, a total of 59 patients had DLQI worsening. About half (31 patients) met the DLQI worsening criterion (4-point increase in total score from baseline), and only a few of them (5 patients) received spesolimab i.v. treatment for flares; 27 patients received spesolimab i.v. treatment and 1 patient received investigator-prescribed SoC to treat GPP worsening without meeting DLQI worsening criterion.

For the time to the first worsening of DLQI up to Week 48, a lower number of patients in the spesolimab low (16 patients), medium (16 patients), and high dose (7 patients) groups than in the placebo group (20 patients) had worsening. The HR was 0.580 (95% CI 0.296, 1.136; nominal p=0.0429) for the low dose, 0.601 (95% CI 0.309, 1.168; nominal p=0.0476) for the medium dose, and 0.259 (95% CI 0.109, 0.620; nominal p=0.0010) for the high dose (See Table 14 below).

The separation of the estimated probability of DLQI worsening between spesolimab groups and placebo started in the first 8 weeks after randomization. The separation between the high dose and placebo was maintained up to Week 48, with no event of worsening reported in the high dose group after 8 weeks. A lower number of patients in all spesolimab groups than in the placebo group reported DLQI worsening up to Week 48 (FIG. 4C).

TABLE 14
Time to the first worsening of DLQI up to Week 48

| | | | Spesolimab | | | | | |
|---|---|---|---|---|---|---|---|---|
| | | | low dose | | medium dose | | high dose | |
| | | | (LD 300 mg, | | (LD 600 mg, | | (LD 600 mg, | |
| | Placebo | | 150 mg q12w) | | 300 mg q12w) | | 300 mg q4w) | |
| Number of patients, N (%) | 31 | (100.0) | 31 | (100.0) | 31 | (100.0) | 30 | (100.0) |
| Patients with DLQI | 20 | (64.5) | 16 | (51.6) | 16 | (51.6) | 7 | (23.3) |
| worsening, N (%) | | | | | | | | |
| Meeting DLQI | 6 | (19.4) | 11 | (35.5) | 10 | (32.3) | 4 | (13.3) |
| worsening criterion | | | | | | | | |
| Received spesolimab i.v. | 1 | (3.2) | 2 | (6.5) | 2 | (6.5) | 0 | |
| Spesolimab i.v. | 14 | (45.2) | 5 | (16.1) | 6 | (19.4) | 2 | (6.7) |

| treatment as a rescue | | | | | |
|---|---|---|---|---|---|
| medication (without | | | | | |
| meeting DLQI | | | | | |
| worsening criteria) | | | | | |
| Investigator- | 0 | 0 | 0 | 1 | (3.3) |

| | | | | |
|---|---|---|---|---|
| prescribed SoC to | | | | |
| treat GPP worsening | | | | |
| Event time [week], 25^{th} | 3.3 (1.6, | 5.6 (3.0, | 6.4 (3.0, | n.c. (2.4, |
| percentile (95% CI) ¹ | 5.9) | 11.0) | 36.0) | n.c.) |
| Probability of event at | 0.645 (0.481, | 0.533 (0.370, | 0.495 (0.334, | 0.247 (0.126, |
| Week 48 (Day337), KM | 0.806) | 0.716) | 0.683) | 0.451) |
| estimate (95% CI) | | | | |
| HR for the time to the | | 0.580 (0.296, | 0.601 (0.309, | 0.259 (0.109, |
| first worsening vs | | 1.136) | 1.168) | 0.620) |
| placebo (95% CI) ² | | | | |
| p-value ³ | | 0.0429 | 0.0476 | 0.0010 |
| | | (nominal) | (nominal) | (nominal) |

DLQI worsening criterion: 4-point increase in total score from baseline up to Week 48. The use of spesolimab i.v. treatment as a GPP flare treatment medication or investigator prescribed SoC to treat GPP worsening were considered as onset of DLQI worsening.
Patients below the age of 16 years (i.e., 12 to 15 years); 1 patient each in spesolimab low dose and high dose groups, did not have to complete the DLQI questionnaire, and were censored at Day 1. No other patient had a baseline value missing or higher than 26 (maximum score possible is 30).
¹ KM estimate of the time when 25% of the patients had endpoint event.
² Cox regression model stratified by the use of systemic GPP medications at randomization.
³ Log-rank test stratified by the use of systemic GPP medications at randomization.

### D. Sustained Remission Up to Week 48:

Sustained remission was defined as a patient with a GPPGA score of 0 or 1 (clear or almost clear) at all visits up to Week 48, without intake of GPP flare treatment medication, or investigator prescribed SoC.

The proportion of patients with sustained remission was the proportion of patients with sustained remission was numerically higher in the spesolimab groups: 0.516 for the spesolimab low dose, 0.452 for the medium dose, 0.633 for the high dose, compared with 0.290 for placebo. The risk difference vs placebo was 0.246 (95% CI 0.013, 0.478) for the spesolimab low dose, 0.166 (95% CI −0.069, 0.401) for the medium dose, and 0.345 (95% CI 0.099, 0.591) for the high dose The results using more stringent definitions were comparable for spesolimab high dose vs placebo. (Table 15, below).

TABLE 15
The proportion of patients with sustained remission up to Week 48

| | | Spesolimab | | | | | |
|---|---|---|---|---|---|---|---|
| | | low dose | | medium dose | | high dose | |
| | | (LD 300 mg, | | (LD 600 mg, | | (LD 600 mg, | |
| | Placebo | 150 mg q12w) | | 300 mg q12w) | | 300 mg q4w) | |
| Number of patients, N (%) | 31 (100.0) | 31 | (100.0) | 31 | (100.0) | 30 | (100.0) |
| Patients with sustained | 9 (29.0) | 16 | (51.6) | 14 | (45.2) | 20 | (66.7) |
| remission at all available | | | | | | | |
| visits, N (%) before | | | | | | | |
| imputation | | | | | | | |
| With sustained remission | 8 (25.8) | 12 | (38.7) | 10 | (32.3) | 12 | (40.0) |

| | | | | |
|---|---|---|---|---|
| at all visits up to Week 48, | | | | |
| N (%) | | | | |
| Proportion with sustained | 0.290 (0.161, | 0.516 (0.348, | 0.452 (0.292, | 0.633 (0.471, |
| remission(95% CI) | 0.466) | 0.680) | 0.622) | 0.770) |
| Risk difference for sustained | | 0.246 (0.013, | 0.166 (−0.069, | 0.345 (0.099, |
| remission vs placebo (95% CI) | | 0.478) | 0.401) | 0.591) |

Landmark analysis showed a lower proportion of patients with loss of sustained remission in the spesolimab high dose group than placebo, which was evident at Week 12 and continued through Week 48. (Data not shown).

### Example 7. Further Endpoints

A. Modified sustained remission: Other definitions for sustained remission were analyzed as a further endpoint (“modified sustained remission”) and sustained pustule clearance (pustulation subscore of 0) and sustained complete clear skin (GPPGA total score of 0 starting from week 8) were analyzed as post-hoc. “Modified sustained remission” is defined as subjects with a GPPGA total score of 0 or 1, and each GPPGA subscore less than or equal to 2 at all visits up to Week 48, without intake of rescue medication, or investigator prescribed SoC (added via TSAP).

The results (Table 16) show the proportion of patients with other definitions of sustained remission were numerically higher for spesolimab high dose than placebo.

TABLE 16
The proportion of patients with other definitions for sustained remission up to Week 48

| | | Spesolimab | | |
|---|---|---|---|---|
| | | low dose | medium dose | high dose |
| | | (LD 300 mg, | (LD 600 mg, | (LD 600 mg, |
| | Placebo | 150 mg q12w) | 300 mg q12w) | 300 mg q4w) |
| Number of patients, N (%) | 31 (100.0) | 31 (100.0) | 31 (100.0) | 30 (100.0) |
| Proportion with GPPGA score | 0.290 (0.161, | 0.484 (0.320, | 0.419 (0.264, | 0.604 (0.444, |
| of 0 or1 and each subscore ≤2 | 0.466) | 0.652) | 0.592) | 0.744) |
| at all visits up to Week | | | | |
| 48 ¹ (95% CI) | | | | |
| Risk difference vs placebo | | 0.215 (−0.017, | 0.132 (−0.105, | 0.316 (0.070, |
| (95% CI) | | 0.447) | 0.369) | 0.561) |
| Proportion with GPPGA | 0.032 (0.006, | 0.000 (0.000, | 0.129 (0.051, | 0.210 (0.109, |
| score of 0 at all visits from | 0.162) | 0.110) | 0.289) | 0.365) |
| Week 8 up to Week 48 ² (95% CI) | | | | |
| Risk difference vs placebo | | −0.035 (−0.100, | 0.096 (−0.038, | 0.176 (0.009, |
| (95% CI) | | 0.030) | 0.230) | 0.343) |
| Proportion with pustulation | 0.258 (0.137, | 0.484 (0.320, | 0.452 (0.292, | 0.636 (0.476, |
| subscore of 0 at all visits | 0.432) | 0.652) | 0.622) | 0.771) |
| from Week 4 up to Week 48 ² | | | | |
| (95% CI) | | | | |
| Risk difference vs placebo | | 0.241 (0.010, | 0.196 (−0.036, | 0.379 (0.138, |
| (95% CI) | | 0.472) | 0.429) | 0.619) |

¹ Further endpoint “modified sustained remission”
² Post hoc analysis

### B. GPPGA Pustulation Subscore of 0:

Two post hoc analyses supported sustained remission. The first measured the proportion with GPPGA pustulation subscore of 0 at all visits from Week 4 up to Week 48 (See block three in Table 16). For sustained remission up to Week 48, the proportion of patients with sustained remission according to this measure was numerically higher in the spesolimab groups (0.484 for the low dose, 0.452 for the medium dose, and 0.636 for the high dose) than in the placebo group (0.258). The risk difference vs placebo was 0.241 (95% CI 0.0101, 0.472) for the spesolimab low dose, 0.196 (95% CI −0.036, 0.429) for the medium dose, and 0.379 (95% CI 0.138, 0.619) for the high dose. The results using more stringent definitions were therefore comparable for spesolimab high dose vs placebo.

### C. GPPGA Total Score 0:

The second analyses measure the proportion with GPPGA score of 0 at all visits from Week 8 up to Week 48 (See block two in Table 16). The proportion of patients with sustained remission according to this measure was numerically higher in the spesolimab medium and high groups (0.129 for the medium dose, and 0.210 for the high dose) than in the placebo group (0.032). The risk difference vs placebo 0.096 (95% CI −0.038, 0.230) for the medium dose, and 0.176 (95% CI 0.009, 0.343) for the high dose.

D. DLQI of 0 or 1 at all Visits Up to Week 48 and without Intake of Rescue Medication or Investigator Prescribed SoC

The proportion of patients first achieving this endpoint was higher in the spesolimab high dose group compared with the placebo group. There were only small differences between spesolimab low and medium dose groups and placebo (Table 17).

TABLE 17
The proportion of patients with DLQI of 0 or 1 at all visits up to Week
48 and without intake of rescue medication or investigator prescribed SoC

| | | | Spesolimab | | | |
|---|---|---|---|---|---|---|
| | | | low dose | medium dose | | high dose |
| | | | (LD 300 mg, | (LD 600 mg, | | (LD 600 mg, |
| | Placebo | | 150 mg q12w) | 300 mg q12w) | | 300 mg q4w) |
| Number of patients, N (%) | 31 | (100.0) | 30 (100.0) | 31 | (100.0) | 29 (100.0) |
| Patients with endpoint, N | 1 | (3.2) | 3 (10.0) | 3 | (9.7) | 7 (24.1) |
| (%) before imputation | | | | | | |

| | | | | |
|---|---|---|---|---|
| Proportion with endpoint | 0.032 (0.006, | 0.100 (0.035, | 0.097 (0.033, | 0.241 (0.122, |
| (95% CI) | 0.162) | 0.256) | 0.249) | 0.421) |
| Risk difference vs placebo | | 0.070 (−0.051, | 0.072 (−0.042, | 0.209 (0.041, |
| (95% CI) | | 0.191) | 0.186) | 0.378) |

Summary: Further analyses for the randomized maintenance period up to Week 48 using DLQI of 0 or 1 at all visits, the proportion of patients whose quality of life was not affected by the disease was higher in the spesolimab high dose group than placebo

Based on the time to the first GPP flare, worsening of PSS, and worsening of DLQI, the effect of the two spesolimab loading doses (300 mg and 600 mg) in the first 4 weeks of the randomized maintenance period was similar to each other and better than placebo.

The geometric mean for spesolimab indicates that spesolimab plasma concentrations were dose related. After any spesolimab treatment (s.c. or i.v.), the percentage of ADA-positive patients in this trial was 45%, 68%, and 41% for patients initially randomized to spesolimab low, medium, and high dose. The percentage of NAb-positive patients (who were all ADA positive) was 45%, 68%, and 34% for the low, medium, and high dose, after any spesolimab treatment. The median onset of ADA ranged from 8.0 to 10.6 weeks, and the time to the maximum titer ranged from 17.1 to 22.6 weeks. No obvious correlation between ADA or NAb development and spesolimab's treatment effect in terms of GPP flare occurrence, flare onset time, or sustained remission in the randomized maintenance period was observed (data not shown).

For the 32 patients who received spesolimab OL 900 mg i.v. doses to treat their flares, the probability of a response after a week was 0.554 (95% CI 0.388, 0.734). Within 2 weeks, 9 patients (0.594, 95% CI 0.423, 0.745) achieved a response. For those who subsequently entered the OL maintenance period with spesolimab s.c. 300 mg (20 patients), their status appeared to be stable over the period analyzed; 9 patients intensified the dosing interval from q12w to q4w.

Non-flaring patients receiving spesolimab s.c. or placebo showed stable CRP, neutrophil, and white blood cell count levels over time, which were within the normal range during the randomized treatment period. Albumin levels were within the normal range throughout the trial. After a flare and OL spesolimab i.v. treatment, elevated CRP, neutrophil, and white blood cell counts rapidly decreased over time after spesolimab i. flare treatment and reached normal levels by Week 2 that were sustained afterwards.

### Example 8: The Occurrence of Treatment Emergent Adverse Events

Safety was assessed based on analyses of adverse events (AEs, including serious AEs (SAEs) and AEs of special interest (AESIs)), physical examination, vital sign recordings, laboratory tests, and 12-lead electrocardiograms (clinically relevant findings were to be recorded as AEs).

TABLE 18
Summary of Adverse Events Within the Randomized Treatment
Period Up to the First Administration of OL spesolimab.

| | Spesolimab SC | | | | | | | | | |
|---|---|---|---|---|---|---|---|---|---|---|
| | Low | | Medium | | High | | Total | | Placebo* | |
| | (N = 32) | | (N = 31) | | (N = 30) | | (N = 93) | | (N = 30) | |
| Patients | n (%) | Rate^{†} | n (%) | Rate^{†} | n (%) | Rate^{†} | n (%) | Rate^{†} | n (%) | Rate^{†} |
| Any AE | 29 | 398.7 | 29 | 411.0 | 26 | 338.2 | 84 | 381.5 | 26 | 414.5 |
| | (90.6) | | (93.5) | | (86.7) | | (90.3) | | (86.7) | |
| Severe AE: | 6 | 25.4 | 7 | 31.6 | 5 | 21.7 | 18 | 26.2 | 7 | 45.6 |
| RCTC | (18.8) | | (22.6) | | (16.7) | | (19.4) | | (23.3) | |
| Grade 3 or 4 | | | | | | | | | | |
| Investigator- | 14 | 86.7 | 11 | 65.0 | 12 | 76.2 | 37 | 75.8 | 10 | 74.9 |
| defined | (43.8) | | (35.5) | | (40.0) | | (39.8) | | (33.3) | |
| drug-related | | | | | | | | | | |
| AE | | | | | | | | | | |
| AE leading to | 0 | 0 | 2 | 8.9 | 3 | 12.9 | 5 (5.4) | 7.1 | 0 | 0 |
| discontinuat | | | (6.5) | | (10.0) | | | | | |
| ion of study | | | | | | | | | | |
| drug | | | | | | | | | | |
| Serious AE^{‡} | 5 | 21.2 | 1 | 4.4 | 3 | 12.8 | 9 | 12.9 | 1 | 5.8 |
| | (15.6) | | (3.2) | | (10.0) | | (9.7) | | (3.3) | |
| AEs | 0 | 0 | 0 | 0 | 0 | 0 | 0 | 0 | 0 | 0 |
| resulting in | | | | | | | | | | |
| death | | | | | | | | | | |

Most common AE_{S}^{§}

| | | | | | | | | | | |
|---|---|---|---|---|---|---|---|---|---|---|
| Skin and | 17 | 89.7 | 20 | 127.4 | 13 | 69.5 | 50 | 93.7 | 22 | 192.2 |
| subcutaneous | (53.1) | | (64.5) | | (43.3) | | (53.8) | | (73.3) | |
| tissue | | | | | | | | | | |
| disorders | | | | | | | | | | |
| Pustular | 10 | 41.9 | 10 | 47.2 | 3 | 12.7 | 23 | 33.5 | 16 | 95.8 |
| psoriasis | (31.3) | | (32.3) | | (10.0) | | (24.7) | | (53.3) | |
| Psoriasis | 4 | 18.0 | 5 | 24.2 | 4 | 18.0 | 13 | 20.0 | 3 | 19.9 |
| | (12.5) | | (16.1) | | (13.3) | | (14.0) | | (10.0) | |
| Infections | 12 | 71.0 | 11 | 64.1 | 8 | 40.5 | 31 | 57.6 | 10 | 75.2 |
| and | (37.5) | | (35.5) | | (26.7) | | (33.3) | | (33.3) | |
| infestations | | | | | | | | | | |
| Upper | 3 | 12.8 | 6 | 30.2 | 0 | 0 | 9 | 13.4 | 4 | 24.9 |
| respiratory | (9.4) | | (19.4) | | | | (9.7) | | (13.3) | |
| tract | | | | | | | | | | |
| infection | | | | | | | | | | |
| COVID-19 | 2 | 8.4 | 1 | 4.4 | 3 | 13.8 | 6 | 8.8 | 1 | 5.9 |
| | (6.3) | | (3.2) | | (10.0) | | (6.5) | | (3.3) | |
| Urinary | 1 | 4.1 | 0 | 0 | 4 | 18.0 | 5 | 7.2 | 0 | 0 |
| tract | (3.1) | | | | (13.3) | | (5.4) | | | |
| infection | | | | | | | | | | |
| General | 9 | 45.9 | 8 | 43.6 | 8 | 44.8 | 25 | 44.8 | 3 | 19.1 |
| disorders | (28.1) | | (25.8) | | (26.7) | | (26.9) | | (10.0) | |
| and | | | | | | | | | | |
| administration | | | | | | | | | | |
| site | | | | | | | | | | |
| conditions | | | | | | | | | | |
| Injection- | 4 | 18.7 | 4 | 19.3 | 5 | 24.7 | 13 | 20.8 | 1 | 6.1 |
| site erythema | (12.5) | | (12.9) | | (16.7) | | (14.9) | | (3.3) | |
| Investigations | 9 | 48.9 | 5 | 24.3 | 5 | 24.2 | 19 | 31.8 | 6 | 43.2 |
| | (28.1) | | (16.1) | | (16.7) | | (20.4) | | (20.0) | |
| Blood | 4 | 17.9 | 1 | 4.4 | 0 | 0 | 5 | 7.3 | 2 | 12.1 |
| creatine | (12.5) | | (3.2) | | | | (5.4) | | (6.7) | |
| phospho- | | | | | | | | | | |
| kinase | | | | | | | | | | |
| increased | | | | | | | | | | |
| Musculoske | 5 | 23.3 | 3 | 14.5 | 5 | 22.7 | 13 | 20.3 | 3 | 18.2 |
| letal and | (15.6) | | (9.7) | | (16.7) | | (14.0) | | (10.0) | |
| connective | | | | | | | | | | |
| tissue | | | | | | | | | | |
| disorders | | | | | | | | | | |
| Arthralgia | 4 | 17.6 | 1 | 4.6 | 3 | 13.3 | 8 | 11.9 | 1 | 6.0 |
| | (12.5) | | (3.2) | | (10.0) | | (8.6) | | (3.3) | |

*A patient randomized to the placebo group, who accidentally received a single dose of spesolimab 150 mg on Day 1 was assigned to the spesolimab low dose group for the analyses of exposure and safety.
^{†}Per 100 patient-years.
^{‡}Serious AEs in patients receiving spesolimab were hypertensive encephalopathy, encephalitis viral, pneumonia, skin bacterial infection, angioedema, drug eruption, palpitations, breast cancer, cholelithiasis, and pustular psoriasis. Of note, hypertensive encephalopathy was a differential diagnosis of viral encephalitis in the same patient. One patient receiving placebo had multiple sclerosis.
^{§}Most common AEs are those occurring in ≥10% of patients in any trial group, by preferred term.
AE, adverse event;
OL, open label;
RCTC, Rheumatology Common Toxicity Criteria;
SC, subcutaneous

The proportion and incidence rates of patients with any AE, as well as severe, serious, and investigator defined drug-related AEs were comparable between all the dose groups during the randomized maintenance treatment period (Table 16). No AEs leading to death occurred in this trial. AEs that led to discontinuation of spesolimab were pustular psoriasis (2 patients, 2.2%); psoriasis, psoriatic arthropathy, and breast cancer (1 patient, 1.1%, for each AE).

A similar proportion of patients receiving spesolimab (90.3%) and placebo (86.7%) experienced an AE; AE incidence was similar across spesolimab dose groups and did not follow a dose-dependent pattern (Tab. Patients receiving spesolimab (total of all doses) and placebo had a similar incidence of severe AEs (19.4% vs. 23.3%, respectively) and investigator-defined drug-related AEs (39.8% vs. 33-3%, respectively). There were no AEs resulting in death; AEs were mostly non-serious and non-severe. The most common AEs were pustular psoriasis, psoriasis, and injection-site erythema (24.7%, 14.0%, and 14.0% of patients receiving spesolimab vs. 53-3%, 10.0%, and 3-3% receiving placebo, respectively). Infection rates were balanced across treatment groups. A greater proportion of patients receiving spesolimab experienced serious AEs (SAEs) compared with the placebo arm (9.7% vs. 3.3%); SAEs did not follow a dose-dependent pattern with spesolimab. SAEs reported in the high-dose spesolimab group were pustular psoriasis, breast cancer, and cholelithiasis (one patient each). Overall, the safety profile of spesolimab was favorable; infection rate was similar across treatment arms, and there was no indication of increased rates with a higher dose. There were no AEs resulting in death, and no hypersensitivity events leading to treatment discontinuation.

### Example 9: Spesolimab Plasma Exposure Following IV and SC Dosing

in Patients with GPP:
The three dosing regimens in the Effisayil™2 study were selected to test a wide range in exposure, allowing a thorough evaluation of the exposure-response relationship of spesolimab in patients with GPP. Loading doses of 600 or 300 mg were included to evaluate whether either exposure is efficacious in preventing GPP flares if other regimens are discontinued. The dosing intervals of q12w versus q4w were selected to evaluate whether flare prevention could be achieved with a dose administered every 3 months, or whether monthly dosing would be required. Since the incidence of flares in an untreated population is unknown and a high incidence of disease is essential to demonstrate patient benefit from preventative treatment, a placebo regimen was crucial for this study. No active control group is included in the study because there is currently no drug approved for the prevention of GPP flares. In the Effisayil™ 2 study, treatment is administered subcutaneously because patients have clear or almost clear skin at randomization, meaning that different spesolimab exposure is expected to be required than is needed for treatment of a flare. In addition, because SC dosing is often preferred by patients, it has the potential to improve participant adherence. In the Effisayil™1 study, an IV dose of 900 mg of spesolimab was shown to be effective in the treatment of GPP flares, with an acceptable safety profile and so was selected as the dose for administration if a patient experiences a GPP flare during the Effisayil™2 study and was used as the comparative dose for the exposure studies.

To simulate the PK of IV vs SC doses of spesolimab to compare drug exposure profiles and support dosing recommendations in patients with GPP, a population PK model was developed using individual-level PK, ADA, and covariate data from 18 studies in which subjects were treated with IV or SC spesolimab.

The mathematical model quantified the PK of spesolimab following IV and SC administration, including the effect of patient-specific factors on PK (e.g., body weight, disease state, ADA titer). The resulting population PK model was used to simulate concentration-time profiles over 12 weeks (84 days) of various IV and SC doses: IV spesolimab 300 mg and 900 mg administered over 90 minutes, as 1 dose or 900 mg as 2 doses (1 week apart), and—SC spesolimab 300 mg, 600 mg, 900 mg and ˜2250 mg injections, as 1 dose or as 2 doses (1 week apart). For each dose, Cmax, Tmax, and AUC over 14 and 84 days were summarized.

PK data from this simulation in GPP patients demonstrate that treatment with IV and SC spesolimab can result in differences in drug exposure in clinical practice. Significantly higher Cmax and more rapid Tmax was observed for the IV vs SC doses of spesolimab. To match the Cmax of 900 mg IV dose, a theoretical SC dose 2.5× greater (2250 mg, equivalent to 15 injections of the 150 mg SC pre-filled syringe) would be required. The immediate and high bioavailability of IV spesolimab compared with SC spesolimab are supportive of the use of IV or SC spesolimab in acute GPP flare treatment or prevention dosing strategies, respectively.

Spesolimab plasma concentration-time course for varying doses of SC and IV were simulated from the PK model for a typical GPP subject, assuming body weight of 75 kg, ADA negative, SC injection into the abdomen, and reference values for all other covariates. The simulations are presented in FIGS. 5A-E. FIG. 5A. is a graphic representation of the model-predicted concentration-time profiles for 300 mg delivered IV versus 300 mg delivered s.c over a period of 12 weeks. The simulated Cmax was approximately 2.5-fold greater with 300 mg IV vs 300 mg SC spesolimab. The Tmax was at the end of the 90-minute infusion for IV spesolimab vs approximately 1 week after dosing for SC spesolimab. FIG. 5B. is a graphic representation of the model-predicted concentration-time profiles of 900 mg IV vs 600 mg SC spesolimab. The simulated Cmax was 3.7-fold greater with 900 mg IV vs 600 mg SC. The Tmax was at the end of the 90-minute infusion for IV spesolimab vs approximately 1 week after dosing for SC spesolimab. FIG. 5C is a graphic representation of the model-predicted concentration-time profiles of 900 mg IV×2 vs 600 mg SC×2 spesolimab. When administered as a 2-dose regimen, exposure increased due to accumulation. However, the Cmax was still 3-fold greater with IV vs SC dosing after the second dose. Similarly, the Tmax occurred approximately 1 week after each dose of SC compared with immediately after the end of each 90-minute IV infusion. FIG. 5D is a graphic representation of the model-predicted concentration-time profiles of 900 mg IV vs 2250 mg SC spesolimab. A SC dose of 2250 mg was required to attain a target Cmax equivalent to that of 900 mg IV spesolimab. With the SC dose, Tmax was delayed 1 week, and AUC was almost twice as large; most importantly a SC injection of this size is not clinically feasible (equivalent to 15 injections of the 150 mg SC pre-filled syringe). FIG. 5E is a summary of exposure metrics after single IV or SC dose in patients with GPP. Simulated spesolimab exposures demonstrated that the Cmax and AUC of the single dose 900 mg IV route of administration consistently exceeded that of all feasible single doses of SC spesolimab. A similar trend was observed for the IV and SC 2-dose regimens. Slow absorption is expected with the SC formulation, with Tmax attained immediately following 90-minute infusion for single IV doses vs approximately 1 week after SC injection.

### Example 10: Spesolimab Treatment Controls GPP Disease Characteristics in Both Acute and Chronic Phases

Treatment with 600 mg loading dose followed by 300 mg of subcutaneous spesolimab every 4 weeks was superior to placebo in flare prevention, reducing the risk of a flare by 84% over 48 weeks (p=0.0005). Skin biopsies (5 mm samples) were taken at: visits 2 (baseline) and 14 (Week 48) during the maintenance treatment period; and rescue visits 1, 6 (4 weeks post-flare) and 14 (OL) during rescue treatment. Skin biopsies at baseline (n=15) were obtained with the aim of using half for RNA sequencing and half for immunohistochemistry analysis.

Spesolimab treatment led to clinical improvements and reduced expression of pathogenic genes associated with GPP flares in the skin biopsies of patients. Of the patients included in this biomarker sub-study, ten had multiple biopsies. Six patients received cyclosporine prior to randomization; all had gene expression patterns suggesting low grade inflammation at baseline. In the four patients who flared (randomized to placebo [n=1], low dose [150 mg s.c. q12w] [n=1], and medium dose [300 mg s.c. q12w] [n=2] spesolimab), pathogenic genes related to a GPP flare and/or IL-36 signaling were further upregulated from baseline. Flare treatment with 900 mg IV spesolimab reduced expression of these pathogenic genes in 3/4 patients. In the five patients receiving high-dose SC spesolimab, no new flares occurred, and expression of GPP flare/IL-36-associated genes decreased (or low expression was sustained) between baseline and Week 48. Histopathological analysis of select neutrophilic proteins confirmed low grade inflammation at baseline and mirrored changes in disease activity and clinical improvements.

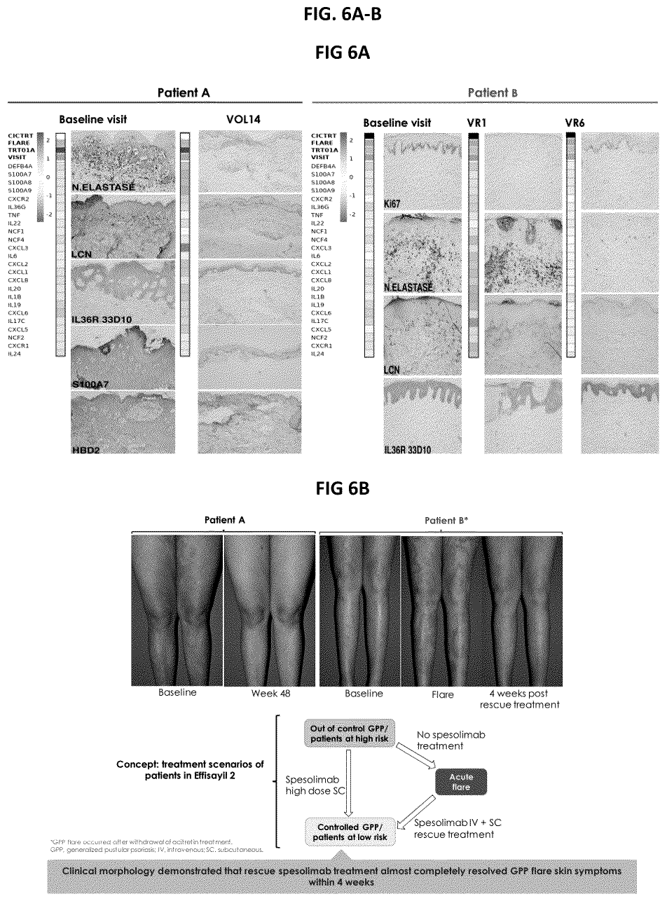

FIGS. 6A and 6B show the exemplary results from two patients, who had received prior therapy for GPP, demonstrating that patients with GPP can be successfully transitioned from background immunosuppressive therapies to maintenance or rescue with spesolimab treatment.

Histopathological analysis of select neutrophilic proteins are shown in FIG. 6A where representative patient A is IL36RN mutation positive, presenting with pustules, scaling, having been treated with cyclosporine before enrollment in the spesolimab trial. Patient A was randomized to receive high dose spesolimab and did not flare throughout the study. Representative patient B did not have IL36RN mutations, had high neutrophil counts, and was treated with acitretin before enrollment. Patient B was randomized to receive placebo and went on to receive rescue spesolimab treatment for a flare during the study.

Pathogenic genes associated with a GPP flare and the IL-36 pathway were differentially expressed (uncorrected p<0.05) between the baseline visit and the first rescue treatment visit (VR1), including IL17C, CXCL6, IL119, IL20, CXCL8, NCF1, IL36G and DEFB4A. Skin biopsies from patients with a tendency for recurrent flares demonstrated higher expression (uncorrected p<0.05) of IL-36 pathway genes and genes associated with GPP flares (IL36G, DEFB4A, CXCL8, CXCL6, 1L19, 1L20, IL17C and NCF1), with fold changes ranging from 1.4 to 5.2, compared with patients with more stable disease. FIG. 6A demonstrates that patients experienced a reduced expression of neutrophilic (neutrophil elastase, lipocalin [LCN]), IL-36R and inflammatory markers (S100A7, human β defensin 2 [hBD2]), indicating reduced inflammation and neutrophilic pustule formation post-spesolimab treatment.

Moreover, as shown in FIG. 6B, clinical morphology mirrored the histopathological changes in both patient A and B, demonstrating that rescue spesolimab treatment almost completely resolved GPP flare skin symptoms. Moreover, Spesolimab treatment prevented GPP flares through suppression of disease activity. Overall, the results suggest spesolimab is more effective in managing GPP than current immunosuppressive therapies.

### Example 11: Micro RNA 223-3p and 223-5p as Biomarkers to Monitor Effects in Skin and Serum in Generalized Pustular Psoriasis after Treatment with Spesolimab

Background: In previous studies it was demonstrated that GPP lesions, compared to non-lesional skin, are associated with elevated expression of IL-36-related transcripts (IL-36a, IL-36b, IL-36g), neutrophil recruitment (CXCL1, CXCL8), pro-inflammatory cytokines (IL-6, IL-19, IL-20), and skin inflammation markers (DEFB4A, S100A7, S100A8, S100A9) which were significantly down-regulated by using a single dose of spesolimab (Baum et al., 2021; Farag et al., unpublished data, 2024). However, the mechanisms leading to specific mRNA expression alterations in GPP are poorly understood.

MicroRNAs (miRNAs) are small non-coding RNA molecules able to post-transcriptionally modulate gene expression and contribute to the development or regulation of several diseases, including skin inflammation (Guo et al. 2010). Only few studies have aimed to describe miRNA expression profiles in the skin of atopic dermatitis patients (Carreras-Badosa et al., 2022), while multiple studies have been performed for psoriasis and/or psoriatic arthritis (Hawkes et al., 2016; Liu et al., 2017; Delic et al., 2020). In addition to tissues, miRNAs have also been found in body fluids, such as serum or plasma, where they demonstrated remarkable value as minimally invasive circulating markers of diseases. Ganguly et al. identified a set of miRNAs which overlapped between skin and serum samples and correlated with disease severity in psoriasis and miR-147b, miR-3614-5p, and miR-125a-5p might be used to distinguish between low severity and the high severity psoriasis patients (Ganguly et al., 2024). In a previous study it has been reported that circulating levels of miR-146a-5p, miR-206, miR338-3p, miR-338-5p, miR-Let 7a, miR-24-1-5p, and miR26a-5p expression was found to be significantly different in patients with hidradenitis suppurativa compared to healthy controls (De Felice et al., 2022). To date there is no information about the role of miRNAs in both skin and blood samples of GPP patients.

Methods: To identify micro RNAs in skin and serum from GPP patients as biomarkers in GPP, microRNA expression profiles were measured using small RNA sequencing and candidate miRNAs were significantly confirmed using qPCR and correlated to clinical outcome.

A total of 25 skin biopsies were available for analysis from a cohort of GPP patients treated with spesolimab in the Effisayil 1 study. A skin punch biopsy (5 mm) was collected at day 1/baseline prior to drug administration (=pre-spesolimab) from lesional (L; n=9) and non-lesional (nL; n=4) skin. Lesional skin biopsies were also collected on day 8 (n=7) after drug administration and at week 8 (n=5) after administration of an additional open-label dose. Due to the limited number of samples available post treatment, the lesional samples collected at day 8 and week 8 post treatment were combined (=post-spesolimab) for analysis. Skin biopsies from 10 healthy controls (50% males; age: 41.9+/−14.0 years) were acquired from Discovery Life Sciences (Kiev, Ukraine).

Biobanked serum samples were obtained from a cohort of 12 GPP patients participating in the EFFISAYIL® 1 study. A total of 25 serum samples were available that were collected at day 1/baseline (n=12) and at week 12 post treatment (n=11). Placebo group was also not included in further analysis due to limited sample size (n=2).

EFFISAYIL® 2 was used as a confirmation study. 41 skin biopsy samples were collected for the following groups: Flaring patients: 5 at baseline, 5 at time of flare, 6 at week 4 post-treatment, 7 at week 52 post treatment; Non-flaring patients: 10 at baseline, 7 at week 52 post patients.

Isolation of total RNA: Skin biopsies were collected in RNA-later® Tissue Protect Tubes (Qiagen, Hilden, Germany) and stored at −20° C. until use. Total RNA, including enrichment of miRNAs, from human skin tissue was extracted using RNeasy Fibrous Tissue Mini Kit (Qiagen, Hilden, Germany). Total RNA, enriched with miRNAs, was isolated from serum samples using miRNeasy Serum/Plasma Advanced Kit (Qiagen, Hilden, Germany) according to the manufacturer's protocol. RNA purity and quantity was assessed by using an Implen NanoPhotometer® N120 (Implen, Munich, Germany).

smallRNA library preparation, sequencing: Sequencing libraries from skin and serum RNA samples were prepared using the QIAseq® miRNA UDI Library Kit (96) (Qiagen, Hilde, Germany) and QIAseq miRNA 96 Index Kit UDI A-H (Qiagen, Hilden, Germany) according to the manufacturer's protocol. Sequencing was performed on the NovaSeq™ 6000 Sequencing System (Illumina) using a NovaSeq 6000 S4 Reagent Kit v1.5 (200 cycles) (Illumina, San Diego, USA) according to the manufacturer's instructions.

Quantitative real-time PCR of microRNA: The TaqMan™ Advanced miRNA cDNA Synthesis Kit (Applied Biosystems, Waltham, MA) was used to reverse transcribe and pre-amplify skin and serum miRNA according to the manufacturer's protocol. RT-qPCR was performed using the TaqMan™ Fast Advanced Master Mix (Applied Biosystems, Waltham, MA), TaqMan Advanced miRNA Assays (Applied Biosystems, Waltham, MA) and the pre-amplified cDNA according to the manufacturer's instructions. RT-qPCR was performed in duplicates on the QuantStudio 7 Flex Real-Time-PCR-System™ (Applied Biosystems, Waltham, MA) according to cycling parameters recommended by the manufacturer. The following assay were used: hsa-miR-223-3p (assay ID: 477983_mir), hsa-miR-223-5p (assay ID: 477984_mir), hsa-miR-1304-3p (assay ID: 479574_mir), has-miR-337-5p (assay ID: 478036_mir), hsa-miR-485-5p (assay ID: 478126_mir), and hsa-miR-361-5p (assay ID: 478056_mir). hsa-miR-361-5p was selected as endogenous control miRNA for normalization after confirming stable expression and low variability using the NormFinder tool and NGS data was selected based on the assessment using “NormFinder_0953” and the recommendation by the vendor. Cycle threshold values (Ct) were determined using QuantStudio™ 6 and 7 Flex Real-Time PCR System Software version 1.7.2. Relative fold changes in gene expression were analyzed according to the 2-ΔΔCt method.

Results: microRNA analyses of samples of non-lesional and lesional skin from GPP patients pre- and post-spesolimab treatment was conducted and the results associated with clinical outcomes in two independent cohorts. The findings in skin biopsies were further correlated with patient matched samples collected from serum to determine whether miRNAs isolated from serum are potential soluble ‘liquid biopsy’ biomarkers as reflections of what is occurring both in the skin and systemically.

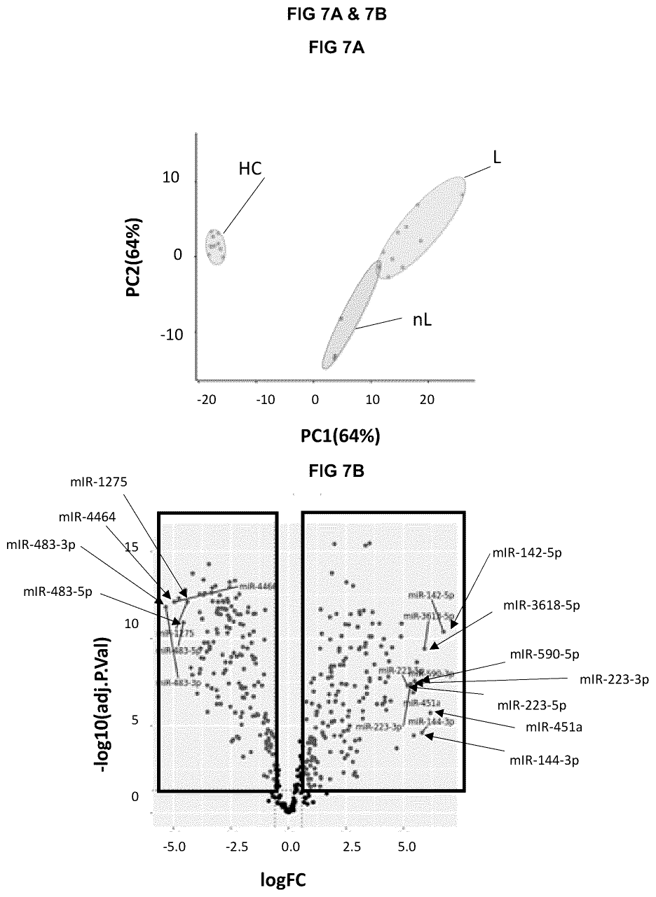

Differentially expressed miRNAs in lesional and non-lesional skin of GPP compared to healthy controls: Differential miRNA expression analysis was performed to explore the differences of the skin miRNA expression profile from GPP patients compared to healthy controls. Overall, 333 miRNAs were significantly (abs. FC≥1.5, adj. P≤0.05) deregulated in lesional skin compared to healthy skin. The expression levels of 173 miRNAs were significantly increased, whereas 160 miRNAs showed significantly lower expression levels in GPP lesions (FIG. 7B).

The strongest upregulation was observed for miR-142-5p, miR-451a and miR-3613-5p with fold changes ranging between 61- to 107-fold. Additionally, among the top 10 upregulated miRNAs, miR-144-3p, miR-944, miR-590-3p, miR-7-5p, miR-142-3p, miR-223-3p and miR-223-5p showed a significant 36- to 56-fold increase in lesional GPP skin compared to healthy controls (FIG. 7B). The strongest downregulation was observed for miR-483-3p, miR-4446, miR-204-3p with a 27- to 40-fold decreased expression in lesional skin of GPP patients compared to healthy controls.

Several of the significantly deregulated miRNAs in lesional GPP skin compared to healthy controls were also deregulated in lesional versus non-lesional GPP skin, but to lower extent (Denis Delid, et al., unpublished data not shown).

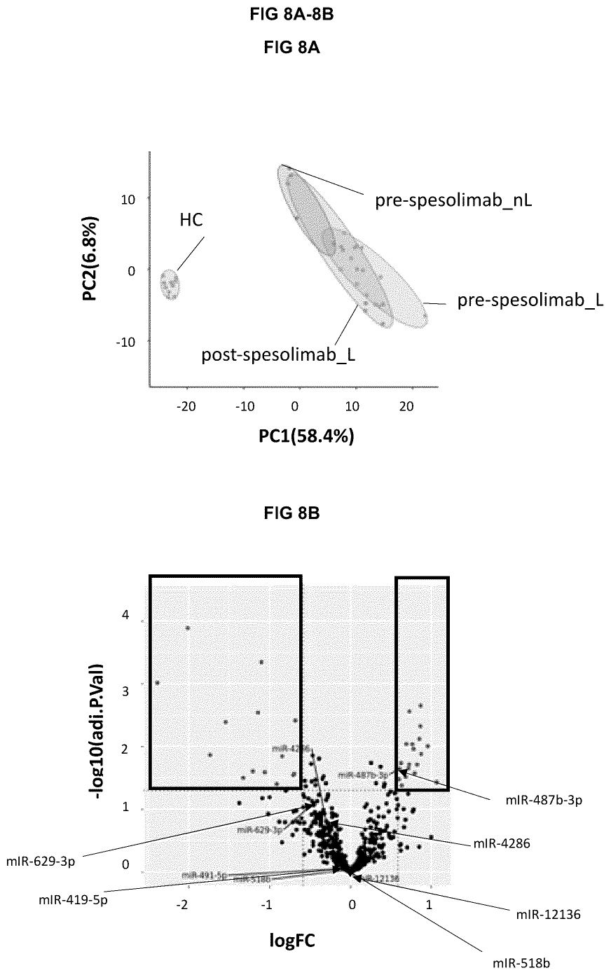

Differentially expressed miRNAs in lesional skin of GPP compared to healthy controls and after spesolimab treatment: To investigate the impact of spesolimab treatment in lesional skin from GPP patients and to identify potential biomarker candidates, small RNA-sequencing was performed on 12 lesional GPP samples following spesolimab treatment. PCA revealed that the skin miRNA profile separated the lesional GPP biopsies derived from subjects treated with spesolimab from healthy controls. The skin miRNA profile did not entirely separate the lesional GPP biopsies post-spesolimab treatment from the baseline biopsies but a shift towards the non-lesional GPP biopsies was identified (FIG. 8A).

Differential expression analysis (abs. FC 1.5, P<0.05) identified 34 deregulated miRNAs in lesional GPP post-spesolimab treatment versus baseline. A total of 14 miRNAs showed lower gene expression levels whereas 20 miRNAs were increased in lesional GPP after spesolimab treatment (FIG. 8B). The strongest decrease was observed for miR-223-3p, miR-223-5p and miR-4455 with 3.3- to 5.2-fold lower expression levels. Within the set of upregulated miRNAs, miR-483-5p, miR-493-5p and miR-485-5p showed the strongest effects with a 1.8- to 2.1-fold increase in expression after spesolimab treatment. The overall effects of spesolimab on differentially expressed miRNAs is summarized in Table 19 below.

TABLE 19
Spesolimab induced effects (>1.5-fold change;
P < 0.05) on miRNA expression in skin

| | logFC.Speso_L_{—} | | P.Value.Speso_L_{—} |
|---|---|---|---|
| miRNAs | nonBL.Speso_L_BL | FC | nonBL.Speso_L_BL |
| hsa-miR-223-3p | −2.4 | −5.2 | 9.7E−04 |
| hsa-miR-223-5p | −2.0 | −4.0 | 1.3E−04 |
| hsa-miR-4455 | −1.7 | −3.3 | 1.4E−02 |
| hsa-miR-4772-3p | −1.5 | −2.9 | 4.1E−03 |
| hsa-miR-142-3p | −1.3 | −2.5 | 3.2E−02 |
| hsa-let-7a-3p | −1.2 | −2.3 | 2.5E−02 |
| hsa-miR-4451 | −1.1 | −2.2 | 2.9E−03 |
| hsa-miR-3614-5p | −1.1 | −2.1 | 4.5E−04 |
| hsa-miR-142-5p | −1.1 | −2.1 | 2.6E−02 |
| hsa-miR-1268b | −0.9 | −1.9 | 4.0E−02 |
| hsa-miR-194-5p | −0.8 | −1.8 | 1.4E−02 |
| hsa-miR-16-2-3p | −0.7 | −1.6 | 2.9E−02 |
| hsa-miR-1304-3p | −0.7 | −1.6 | 2.7E−02 |
| hsa-miR-7-1-3p | −0.7 | −1.6 | 3.9E−03 |
| hsa-miR-483-5p | 1.1 | 2.1 | 3.7E−02 |
| hsa-miR-493-5p | 1.0 | 1.9 | 9.9E−03 |
| hsa-miR-485-5p | 0.9 | 1.8 | 1.3E−02 |
| hsa-miR-127-3p | 0.9 | 1.8 | 2.2E−03 |
| hsa-miR-432-5p | 0.9 | 1.8 | 4.8E−03 |
| hsa-miR-382-5p | 0.9 | 1.8 | 7.6E−03 |
| hsa-miR-493-3p | 0.8 | 1.8 | 2.0E−02 |
| hsa-miR-127-5p | 0.8 | 1.7 | 2.7E−02 |
| hsa-miR-409-3p | 0.8 | 1.7 | 1.1E−02 |
| hsa-let-7c-3p | 0.8 | 1.7 | 9.2E−03 |
| hsa-miR-887-3p | 0.7 | 1.7 | 2.8E−03 |
| hsa-miR-409-5p | 0.7 | 1.7 | 1.9E−02 |
| hsa-miR-369-5p | 0.7 | 1.6 | 2.2E−02 |
| hsa-miR-134-5p | 0.7 | 1.6 | 9.2E−03 |
| hsa-miR-323a-3p | 0.7 | 1.6 | 3.1E−02 |
| hsa-miR-337-5p | 0.6 | 1.6 | 4.2E−02 |
| hsa-miR-299-5p | 0.6 | 1.5 | 1.8E−02 |
| hsa-miR-379-5p | 0.6 | 1.5 | 2.3E−02 |
| hsa-miR-154-5p | 0.6 | 1.5 | 3.3E−02 |
| hsa-miR-487b-3p | 0.6 | 1.5 | 2.3E−02 |

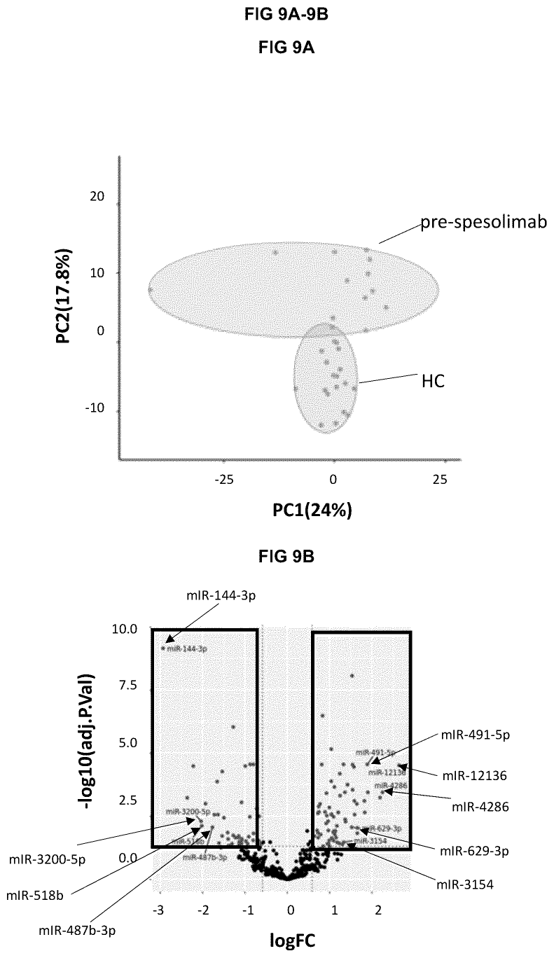

Differentially expressed miRNAs in serum of GPP compared to healthy controls: Minimally/non-invasive, serum miRNA biomarkers were investigated to determine whether changes observed in skin also translated to the periphery. Small RNA-seq was performed on serum samples obtained from 12 GPP patients and 20 healthy controls. PCA of the serum miRNA expression profiles clearly separated the GPP patients from healthy volunteers, which indicated substantial differences in the systemic miRNA profile of GPP patients (FIG. 9A).

Compared to healthy volunteers, a total of 114 serum miRNAs were significantly (abs. FC≥1.5, adj. P≤0.05) deregulated in GPP patients. Among them, 69 miRNAs showed upregulated miRNA expression levels, whereas 45 miRNAs showed downregulated expression levels in GPP patients (FIG. 9B). The top deregulated miRNAs included miR-12136, miR-4286 and miR-29c-5p with a 4.6- to 6.3-fold increase and miR-144-3p, miR-299-3p and miR-375-3p with a 4.7- to 7.6-fold decrease in GPP patients compared to healthy serum.

Differentially expressed miRNAs in serum of GPP after spesolimab treatment: The treatment effect on the serum miRNA profile was investigated to assess whether the treatment effect observed in skin is also reflected in serum and if serum miRNAs may serve as minimally/non-invasive biomarker candidates for monitoring treatment of GPP patients including, but not limited to: detecting the presence or absence of a beneficial response in a GPP patient after administration of an anti-interleukin-36 receptor antibody, determining whether a potential therapeutic agent is efficacious in the treatment and prevention of GPP, determining whether to initiate treatment of the subject, modify the treatment dose, modify the dosing interval, or discontinue treatment, monitoring patient response to a GPP treatment, and patient compliance with the treatment protocol. Small RNA-sequencing was performed on 11 serum samples from GPP patients who received spesolimab treatment.

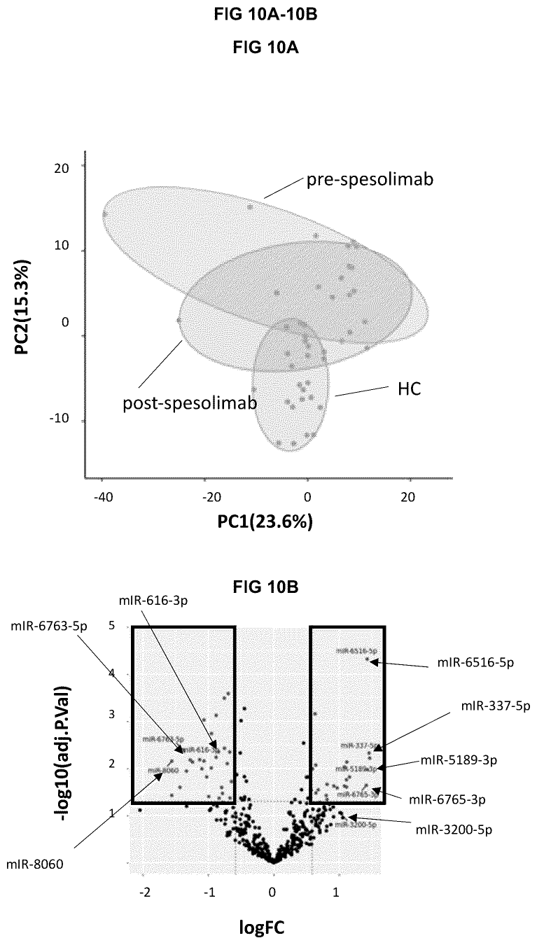

PCA analysis of the filtered serum miRNA expression profiles showed that the samples from GPP patients after spesolimab treatment clustered between the baseline and healthy control samples (FIG. 10A).

Differential miRNA expression analysis revealed that 50 miRNAs showed an altered expression (1.5-fold; P<0.05) compared to baseline, including 21 upregulated and 29 downregulated miRNAs (see Table 20). The most strongly deregulated serum miRNAs, including miR-376b-3p, miR-337-5p and miR-5189-3p, exhibited a 2.7- to 2.8-fold increase whereas miR-16-5p, miR-8060 and miR-8085 showed a 2.7- to 2.9-fold decrease in serum levels post-spesolimab treatment compared to baseline (FIG. 10B).

TABLE 20
Spesolimab induced effects (>1.5-fold change;
P < 0.05) on miRNA expression in serum

| miRNAs | logFC | FC | P.Value |
|---|---|---|---|
| hsa-miR-16-5p | −1.6 | −2.9 | 3.7E−02 |
| hsa-miR-8060 | −1.6 | −2.9 | 7.0E−03 |
| hsa-miR-8085 | −1.4 | −2.7 | 2.5E−02 |
| hsa-miR-6763-5p | −1.4 | −2.6 | 4.3E−03 |
| hsa-miR-4644 | −1.3 | −2.5 | 1.1E−02 |
| hsa-miR-199b-5p | −1.3 | −2.4 | 6.6E−03 |
| hsa-miR-629-3p | −1.2 | −2.4 | 7.3E−03 |
| hsa-miR-616-3p | −1.1 | −2.2 | 6.5E−03 |
| hsa-miR-330-3p | −1.1 | −2.1 | 1.0E−02 |
| hsa-miR-542-5p | −1.1 | −2.1 | 7.0E−03 |
| hsa-miR-197-3p | −1.1 | −2.1 | 9.4E−04 |
| hsa-miR-365a-3p | −1.0 | −2.0 | 1.5E−02 |
| hsa-miR-1277-5p | −1.0 | −2.0 | 4.0E−02 |
| hsa-miR-3178 | −1.0 | −1.9 | 9.8E−03 |
| hsa-miR-574-3p | −1.0 | −1.9 | 1.8E−03 |
| hsa-miR-223-5p | −0.9 | −1.9 | 3.0E−03 |
| hsa-miR-223-3p | −0.9 | −1.8 | 5.8E−03 |
| hsa-miR-197-5p | −0.9 | −1.8 | 7.5E−04 |
| hsa-miR-145-5p | −0.8 | −1.8 | 4.4E−03 |
| hsa-miR-4492 | −0.8 | −1.8 | 4.3E−02 |
| hsa-miR-7704 | −0.8 | −1.7 | 2.5E−02 |
| hsa-miR-3928-3p | −0.8 | −1.7 | 3.4E−02 |
| hsa-miR-424-5p | −0.8 | −1.7 | 3.2E−04 |
| hsa-miR-143-3p | −0.8 | −1.7 | 3.7E−03 |
| hsa-miR-1304-3p | −0.7 | −1.6 | 7.9E−03 |
| hsa-miR-191-5p | −0.7 | −1.6 | 2.6E−04 |
| hsa-miR-424-3p | −0.7 | −1.6 | 4.5E−03 |
| hsa-miR-491-5p | −0.7 | −1.6 | 4.3E−02 |
| hsa-miR-93-3p | −0.6 | −1.6 | 1.9E−02 |
| hsa-miR-376b-3p | 1.5 | 2.8 | 6.1E−03 |
| hsa-miR-337-5p | 1.5 | 2.7 | 4.7E−03 |
| hsa-miR-5189-3p | 1.4 | 2.7 | 1.1E−02 |
| hsa-miR-6516-5p | 1.4 | 2.7 | 4.8E−05 |
| hsa-miR-6765-3p | 1.4 | 2.7 | 2.3E−02 |
| hsa-miR-1185-1-3p | 1.2 | 2.2 | 1.5E−02 |
| hsa-miR-181d-5p | 1.1 | 2.2 | 7.3E−03 |
| hsa-miR-485-5p | 1.1 | 2.2 | 1.7E−02 |
| hsa-miR-431-5p | 1.1 | 2.2 | 2.5E−02 |
| hsa-miR-548ax | 1.1 | 2.1 | 9.4E−03 |
| hsa-miR-5189-5p | 1.1 | 2.1 | 2.4E−02 |
| hsa-miR-375-3p | 1.0 | 2.0 | 2.6E−02 |
| hsa-miR-362-5p | 0.9 | 1.9 | 2.4E−02 |
| hsa-miR-100-5p | 0.8 | 1.8 | 2.2E−02 |
| hsa-miR-1306-3p | 0.8 | 1.8 | 4.6E−02 |
| hsa-miR-1246 | 0.8 | 1.7 | 3.8E−02 |
| hsa-miR-195-5p | 0.7 | 1.6 | 2.8E−02 |
| hsa-miR-181b-5p | 0.6 | 1.6 | 3.3E−02 |
| hsa-miR-200c-3p | 0.6 | 1.6 | 8.5E−03 |
| hsa-miR-652-3p | 0.6 | 1.5 | 6.9E−04 |
| hsa-miR-28-5p | 0.6 | 1.5 | 1.0E−02 |

Identification of miRNA core sets and integrated miRNA target mRNA pathway analysis: The comparison of lesional skin biopsies and serum from GPP patients with healthy controls and post-spesolimab treatment identified a set of five core miRNAs that showed a consistent deregulation in GPP and modulation upon spesolimab treatment. This set of miRNAs comprised miR-223-3p, miR-223-5p, miR-1304-3p, miR-485-5p, and miR-337-5p (FIG. 11A) and the effects on deregulation after spesolimab in skin and serum are summarized in FIG. 11B.

To further understand the mechanistic understanding of the identified miRNA biomarker set, a target prediction analysis of the candidate miRNAs was performed using MirTarBase. Target mRNAs were selected from the differential gene expression analysis in skin obtained from the matched patient data in EFFISAYIL® 1 (Farag et al., 2024) and pathway analysis was conducted using the REACTOME and HALLMARK data sets obtained from MSigDB. Significant enrichment of pathways was observed for miR-223-3p which includes key signaling pathways in inflammatory processes (unpublished data).

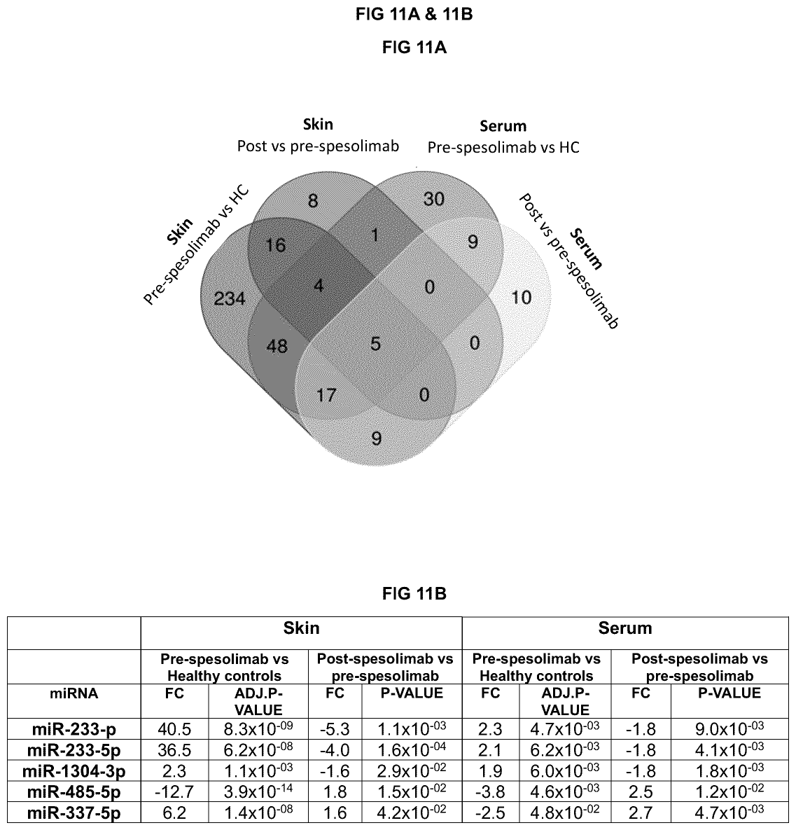

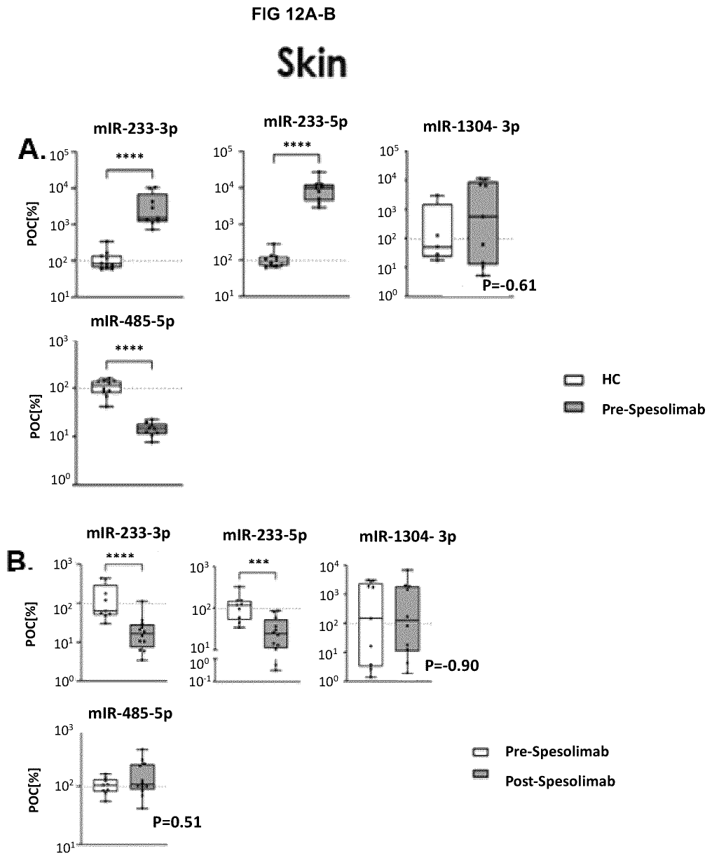

Confirmation of selected miRNA candidates in GPP and correlation with clinical parameters: To verify the set of miRNA biomarker candidates identified in skin and serum, RT-qPCR was used to confirm the small RNA-seq results. Pairwise comparisons of the data generated by RT-qPCR confirmed the significant deregulation of the biomarker candidates (P<0.0001) in lesional GPP skin compared to healthy controls for all miRNAs, except miR-1304-3p. The strong upregulation of miR-223-3p and miR-223-5p in GPP lesions were confirmed by RT-qPCR, which is represented by median percent of control (POC) values >1,500% to 9,800% and a downregulation in GPP patients was confirmed for miR-485-5p, which is indicated by median POC values between 14% to 32% (FIG. 12A). Spesolimab treatment significantly (P<0.001) reduced the expression of miR-223-3p and miR-223-5p by 76% to 84%, which is consistent with the small RNA-seq data whereas no significant effects on miR-485-5p level after spesolimab treatment were detected (FIG. 12B).

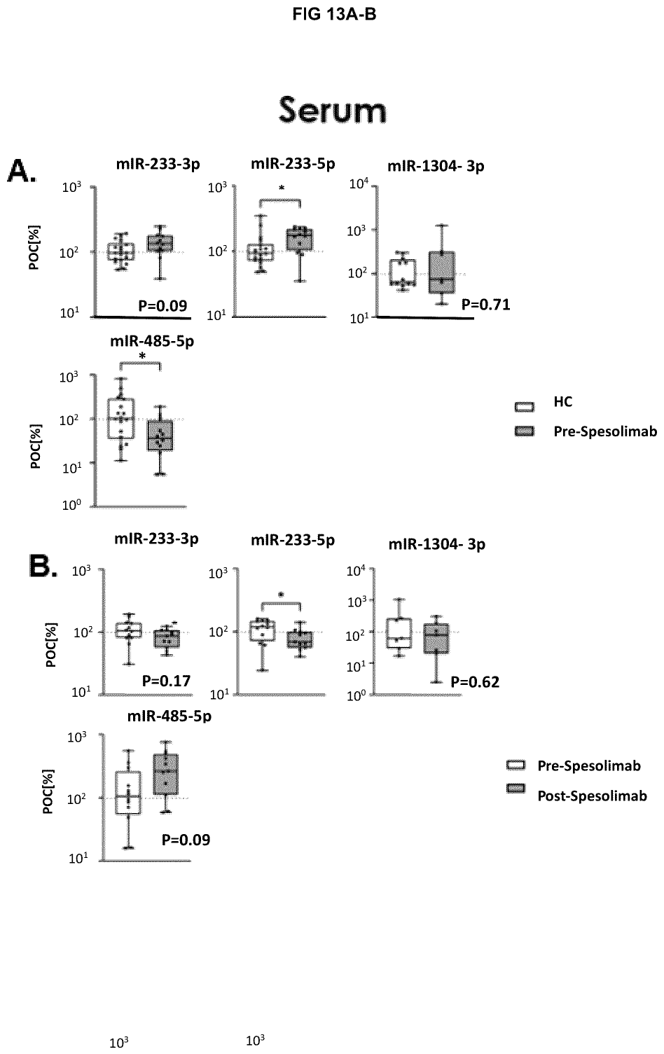

In serum, the RT-qPCR analysis confirmed a significant (P<0.05) upregulation of miRNA-223-5p with a median POC value of 174%, while miR-223-3p showed a trend (P<0.09) towards a 134% upregulation in serum at baseline compared to healthy controls. Significant downregulation in baseline serum was confirmed for miR-485-5p (P<0.05), which is indicated by median POC values ranging between 23% to 48% (FIG. 13A). Spesolimab treatment significantly reduced the expression of miR-223-5p (32%) in serum of GPP patients while the expression levels of miR-223-3p followed the same trend (P=0.17). Taken together miR-223-3p and miR-223-5p resulted as the most promising miRNA candidates to monitor disease progression and treatment effects.

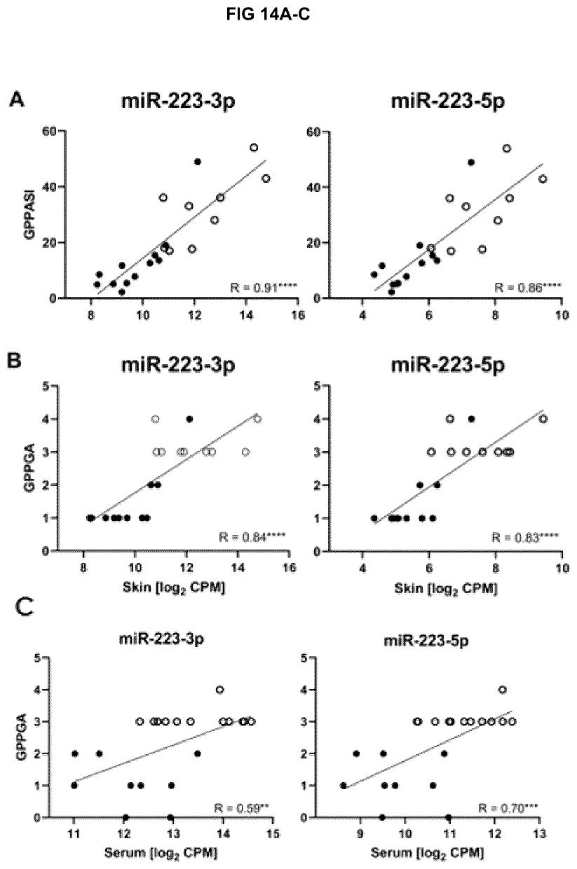

To examine whether variations in skin miRNA expression of the differentially expressed miRNAs can be used to monitor disease progression and treatment response, skin miRNA levels pre- and post-spesolimab treatment were correlated with the GPPASI and GPPGA scores. The correlation analysis demonstrated a highly significant (P<0.0001) positive correlation between the changes in GPPASI and GPPGA scores and the changes in skin expression levels of miR-223-3p (Rs_GPPASI=0.91; Rs_GPPGA=0.84) and miR-223-5p (Rs_GPPASI=0.86; Rs_GPPGA=0.83) after spesolimab treatment (FIG. 14A, B). Correlation analysis between serum miRNA levels and the GPPGA score reveled a significant correlation for miR-223-5p (Rs_GPPGA=0.7; P<0.001) and miR-223-3p (Rs_GPPGA=0.59; P<0.01) (FIG. 14C).

Summary: In skin and serum samples from GPP patients we identified 5 miRNAs, miR-223-3p, miR-223-5p, miR-1304-3p, miR-485-5p and miR-337-5p, which were increased in GPP patients compared to healthy controls and decreased after spesolimab treatment. Specifically, three miRNAs, including miR-223-3p, miR-223-5p and miR-1304-3p were upregulated in the lesional skin and serum of GPP patients and were downregulated following spesolimab treatment. Conversely, two miRNAs, including miR-485-5p, and miR-337-5p were downregulated in GPP patients and were upregulated following spesolimab treatment. Integrated miRNA-mRNA analysis identified that in particular miR-223-3p miRNAs is involved as an upstream regulator of their target mRNAs in multiple pathways contributing to GPP pathophysiology.

MiR-223 has previously been reported to exhibit increased expression levels in various inflammatory disorders such as plaque psoriasis, asthma, chronic obstructive pulmonary disease, and inflammatory bowel disease (Lovendorf et al., 2014; Roffel et al., 2020, Yarani et al., 2022). This study demonstrated an involvement of both arms of miR-223, miR-223-3p and miR-223-5p, in the deregulated molecular mechanisms of GPP. Without wishing to be bound by theory, it is possible that upregulation of miR-223 in GPP patients may mediate an anti-inflammatory effect via negative regulation of the NF-κB signaling cascade, targeting the chemokines CXCL2 and CCL3 (Roffel et al., 2020), both of which are known to recruit immune cells including neutrophils (Filippo et al., 2013; Reichel et al., 2009). The increase of miR-223 may thus represent a counter-regulatory mechanism to mitigate the exaggerated inflammatory response and neutrophil recruitment observed in GPP.

Using qPCR this study confirmed the findings for miR-223-3p and miR-223-5p and demonstrated a significant correlation with GPPASI and GPPGA, suggesting that miR-223-3p and miR-223-5p can be used as minimally/non-invasive biomarkers to monitor GPP disease progression and treatments effects induced by spesolimab.

1. A method of treatment of generalized pustular psoriasis (GPP), including the treatment and prevention of flares in a subject with a history of GPP symptoms and/or with a confirmed history (diagnosis) of GPP based on the consensus diagnostic criteria defined by the ERASPEN Diagnostic Criteria, comprising administering to the subject a first loading dose of 300 mg-600 mg of an anti-IL-36R antibody; followed by maintenance doses of 150 mg-600 mg of said anti-IL-36R antibody administered to the subject at 4 week (q4w) or 12 week (q12w) intervals for 8-48 weeks following the last maintenance dose, wherein said loading and maintenance doses are delivered parenterally.

2. The method of claim 1, wherein said total loading dose comprises 300 mg of anti-IL-36R antibody delivered subcutaneously at week 0 or 1, followed by maintenance doses of 150 mg of said anti-IL-36R antibody administered subcutaneously at 12-week (q12w) intervals.

3. The method of claim 1, wherein said total loading dose comprises 600 mg of anti-IL-36R antibody delivered subcutaneously at week 0 or 1, followed by maintenance doses of 300 mg of said anti-IL-36R antibody administered subcutaneously at 12-week (q12w) intervals.

4. The method of claim 1, wherein in said total loading dose comprises 600 mg of anti-IL-36R antibody delivered subcutaneously at week 0 or 1, followed by maintenance doses of 300 mg of said anti-IL-36R antibody administered subcutaneously at 4-week (q4w) intervals.

5. A method of treatment of generalized pustular psoriasis (GPP) in a subject with a history of GPP when not experiencing a flare, comprising administering to the subject a first loading dose of 300 mg-600 mg of an anti-IL-36R antibody; followed by maintenance doses of 150 mg-600 mg of said anti-IL-36R antibody administered to the subject at 4-week (q4w) or 12-week (q12w) intervals for 8-48 weeks following the last maintenance dose, wherein said loading and maintenance doses are delivered parenterally.

6. The method of claim 5, wherein said total loading dose comprises 300 mg of anti-IL-36R antibody delivered subcutaneously at week 0 or 1, followed by maintenance doses of 150 mg of said anti-IL-36R antibody administered subcutaneously at 12-week (q12w) intervals.

7. The method of claim 5, wherein said total loading dose comprises 600 mg of anti-IL-36R antibody delivered subcutaneously at week 0 or 1, followed by maintenance doses comprising 300 mg of said anti-IL-36R antibody administered subcutaneously at 12-week (q12w) intervals.

8. The method of claim 5, wherein said total loading dose comprises 600 mg of anti-IL-36R antibody delivered subcutaneously at week 0 or 1, followed by maintenance doses of 300 mg of said anti-IL-36R antibody administered subcutaneously at 4-week (q4w) intervals.

9. A method of reducing the incidence, recurrence of, and/or frequency of generalized pustular psoriasis (GPP) flares in a subject with a history of GPP symptoms and/or with a confirmed history (diagnosis) of GPP based on the consensus diagnostic criteria defined by the ERASPEN Diagnostic Criteria, comprising administering to the subject a first loading dose of 300 mg-600 mg of an anti-IL-36R antibody; followed by maintenance doses of 150 mg-600 mg of said anti-IL-36R antibody administered to the subject at 4 week (q4w) or 12 week (q12w) intervals for 8-48 weeks following the last maintenance dose, wherein said loading and maintenance doses are delivered parenterally.

10. The method of claim 9, wherein said total loading dose comprises 300 mg of anti-IL-36R antibody delivered subcutaneously at week 0 or 1, followed by maintenance doses comprising 150 mg of said anti-IL-36R antibody administered subcutaneously at 12-week (q12w) intervals.

11. The method of claim 9, wherein in said total loading dose comprises 600 mg of anti-IL-36R antibody delivered subcutaneously at week 0 or 1, followed by maintenance doses of 300 mg of said anti-IL-36R antibody administered subcutaneously at 12-week (q12w) intervals.

12. The method of claim 9, wherein in said total loading dose comprises 600 mg of anti-IL-36R antibody delivered subcutaneously at week 0 or 1, followed by maintenance dose of 300 mg of said anti-IL-36R antibody administered subcutaneously at 4-week (q4w) intervals.

13. The method of claim 5, wherein in said total loading dose comprises 600 mg of anti-IL-36R antibody delivered subcutaneously at week 0 or 1, followed by maintenance dose of 300 mg of said anti-IL-36R antibody administered subcutaneously at 4-week (q4w) intervals.

14. The method according to claim 1, wherein the subject has a GPP Physician Global Assessment (GPPGA) total score of ≤1 at the start of treatment.

15. The method according to claim 1, wherein the subject has a GPP Physician Global Assessment (GPPGA) total score of ≤1 and a GPPGA pustulation subscore of ≤1 at the start of treatment and/or before the administration of the first loading dose of an anti-IL-36R antibody.

16. The method according to claim 1, wherein the subject has a GPP Physician Global Assessment (GPPGA) total score of ≤1 and a GPPGA pustulation subscore of ≤1 after the administration of at least one maintenance dose of 150 or 300 mg of an anti-IL-36R antibody up to at least 48 weeks following the initial loading dose of 300 mg or 600 mg an anti-IL-36R antibody.

17. The method according to claim 1, wherein the subject has a GPP Physician Global Assessment (GPPGA) total score of ≤1 and a GPPGA pustulation subscore of ≤1 before and after the administration of at least one maintenance dose of 150 or 300 mg of an anti-IL-36R antibody up to at least 48 weeks following the initial loading dose of 300 mg or 600 mg an anti-IL-36R antibody.

18. The method according to claim 1, wherein the administration of an anti-IL-36R antibody to a subject with a history of GPP symptoms, and/or with a confirmed history (diagnosis) of GPP based on the consensus diagnostic criteria defined by the ERASPEN Diagnostic Criteria achieves one or more of the following results:
(a) maintenance of a Generalized Pustular Psoriasis Global Assessment (GPPGA) pustulation subscore of 0 or 1 after administering said anti-IL-36R antibody up week 48 after the initial loading dose;
(b) maintenance of a GPPGA total score of 0 or 1 after administering said anti-IL-36R antibody up to week 48 after the initial loading dose;
(c) a sustained remission of GPP symptoms, defined as a subject treated with said anti-IL-36R antibody who maintains a GPPGA score of 0 or 1 (clear or almost clear) at all visits up to Week 48, without intake of GPP flare medication, or investigator-prescribed Standard of Care (SoC) after administering said anti-IL-36R antibody;
(d) a decrease in the occurrence of GPP flare(s), wherein GPP flare is defined as in increase in GPP Physician Global Assessment (GPPGA) total score of ≥2 and a GPPGA pustulation subscore of ≥2, from baseline up to week 48 after administering said anti-IL-36R antibody; or
(e) a prolongation of the time to a first GPP flare as measured by the time from baseline to the onset of said first GPP flare, wherein GPP flare is defined as in increase in the GPP Physician Global Assessment (GPPGA) total score of ≥2, and a GPPGA pustulation subscore of ≥2, from baseline up to week 48 after administering said anti-IL-36R antibody.

19. The method according to claim 1, wherein the subject experiences a reduced risk of worsening of a Psoriasis Symptom Scale (PSS) score up to Week 48 after receiving the maintenance treatment of anti-IL-36R antibody, wherein worsening is defined as a 4-point increase in total score as compared to the PSS score at baseline in the subject before the administration.

20. The method according to claim 1, wherein the subject experiences a reduced risk of worsening of Dermatology Quality of Life Index (DLQI), up to Week 48 after receiving the maintenance treatment of anti-IL-36R antibody, wherein worsening is defined as a 4-point increase in total score as compared to the DQLI score at baseline in the subject before the administration.

21. A method of treatment of generalized pustular psoriasis (GPP), including the treatment and prevention of flares of a subject with a history of GPP symptoms, and/or with a confirmed history (diagnosis) of GPP based on the consensus diagnostic criteria defined by the ERASPEN Diagnostic Criteria, wherein said subject has a GPPGA total score of ≤1 and a GPPGA pustulation subscore of ≤1 prior to the initiation of treatment, said method comprising the steps of:
(a) administering to the subject a loading dose of 600 mg of an anti-IL-36R antibody at week 0 to 1, followed by maintenance doses comprising 300 mg of an anti-IL-36R antibody administered to the subject at 4-week intervals, wherein administration of the loading dose and the maintenance dose of said anti-IL-36R antibody is delivered subcutaneously; and
(b) assessing the GPP Physician Global Assessment (GPPGA) total and/or GPPGA pustulation subscore of the subject up to at least week 48 after the initial loading dose, wherein the time to the first GPP flare up to Week 48 after the initiation of treatment is defined by a GPPGA pustulation subscore of ≥2 and an increase in GPPGA total score by 2 from baseline.

22. A method of treatment of generalized pustular psoriasis (GPP), including the treatment and prevention of flares of a subject with a history of GPP symptoms, and/or with a confirmed history (diagnosis) of GPP based on the consensus diagnostic criteria defined by the ERASPEN Diagnostic Criteria, wherein said subject has a GPPGA total score of ≤1, and a GPPGA pustulation subscore of ≤1 prior to the initiation of treatment, said method comprising the steps of:
(a) administering to the subject a loading dose of 600 mg of an anti-IL-36R antibody at week 0 to 1, followed by maintenance doses comprising 300 mg of an anti-IL-36R antibody administered to the subject at 12-week intervals, wherein administration of the loading dose and the maintenance dose of said anti-IL-36R antibody is delivered subcutaneously; and
(b) assessing the GPP Physician Global Assessment (GPPGA) total and/or GPPGA pustulation subscore of the subject up to at least week 48 after the initial loading dose, wherein the time to the first GPP flare up to Week 48 after the initiation of treatment is defined by a GPPGA pustulation subscore of ≥2 and an increase in GPPGA total score by 2 from baseline.

23. A method of treatment of generalized pustular psoriasis (GPP), including the treatment and prevention of flares of a subject with a history of GPP symptoms, and/or with a confirmed history (diagnosis) of GPP based on the consensus diagnostic criteria defined by the ERASPEN Diagnostic Criteria, wherein said subject has a GPPGA total score of ≤1 and a GPPGA pustulation subscore of ≤1 prior to the initiation of treatment, said method comprising the steps of:
(a) administering to the subject a loading dose of 300 mg of an anti-IL-36R antibody at week 0 to 1, followed by maintenance doses comprising 150 mg of an anti-IL-36R antibody administered to the subject at 12-week intervals, wherein administration of the loading dose and the maintenance dose of said anti-IL-36R antibody is delivered subcutaneously; and
(b) assessing the GPP Physician Global Assessment (GPPGA) total and/or GPPGA pustulation subscore of the subject up to at least week 48 after the initial loading dose, wherein the time to the first GPP flare up to Week 48 after the initiation of treatment is defined by a GPPGA pustulation subscore of ≥2 and an increase in GPPGA total score by 2 from baseline.

24. The method according to claim 1, wherein the anti-IL-36R antibody comprises: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

25. The method according to claim 1, wherein the anti-IL-36R antibody comprises:
I. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
II. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
III. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
IV. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
V a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).
VI. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

26. The method according to claim 1, wherein the anti-IL-36R antibody comprises:
i. a light chain variable region comprising the amino acid sequence of SEQ ID NO: 77; and a heavy chain variable region comprising the amino acid sequence of SEQ ID NO: 87; or
ii. a light chain variable region comprising the amino acid sequence of SEQ ID NO: 77; and a heavy chain variable region comprising the amino acid sequence of SEQ ID NO: 88; or
iii. a light chain variable region comprising the amino acid sequence of SEQ ID NO: 77; and a heavy chain variable region comprising the amino acid sequence of SEQ ID NO: 89; or
iv. a light chain variable region comprising the amino acid sequence of SEQ ID NO: 80; and a heavy chain variable region comprising the amino acid sequence of SEQ ID NO: 87; or
v. a light chain variable region comprising the amino acid sequence of SEQ ID NO: 80; and a heavy chain variable region comprising the amino acid sequence of SEQ ID NO: 88; or
vi. a light chain variable region comprising the amino acid sequence of SEQ ID NO: 80; and a heavy chain variable region comprising the amino acid sequence of SEQ ID NO: 89; or
vii. a light chain variable region comprising the amino acid sequence of SEQ ID NO: 85; and a heavy chain variable region comprising the amino acid sequence of SEQ ID NO: 100; or
viii. a light chain variable region comprising the amino acid sequence of SEQ ID NO: 85; and a heavy chain variable region comprising the amino acid sequence of SEQ ID NO:101; or
ix. a light chain variable region comprising the amino acid sequence of SEQ ID NO: 86; and a heavy chain variable region comprising the amino acid sequence of SEQ ID NO: 100; or
x. a light chain variable region comprising the amino acid sequence of SEQ ID NO: 86; and a heavy chain variable region comprising the amino acid sequence of SEQ ID NO:101.

27. The method according to claim 1, wherein the anti-IL-36R antibody comprises:
i. a light chain comprising the amino acid sequence of SEQ ID NO: 115; and a
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

28. The method according to claim 1, wherein the anti-IL-36R antibody is spesolimab.

29. A method for detecting the presence or absence of a beneficial response in a GPP patient after administration of an anti-interleukin-36 receptor antibody (anti-IL-36R antibody) according to the method of claim 1, comprising:
(a) obtaining a biological sample from the patient pre-treatment and post-treatment after administration of the anti-IL-36R antibody;
(b) measuring in each sample the level of one or more biomarkers, or the level of expression of one or more biomarkers in each sample pre-treatment and post-treatment;
(c) comparing the pre-treatment level to post-treatment level of the biomarkers; and
(d) determining the difference in levels between the pre-treatment sample and the post-treatment sample reflects a beneficial response in the patient, wherein the one or more biomarkers comprise microRNAs selected from the group consisting of miR-223-3p, miR-223-5p, miR-1304-3p, miR-485-5p, or miR-337-5p,
wherein downregulation or upregulation post-treatment as compared to pre-treatment baseline, indicates beneficial response.

30. The method of claim 29, wherein the biological sample is a skin biopsy, blood, plasma, or serum sample.

31. The method of claim 29, wherein the one or more of microRNAs are miR-223-5p or miR-223-3 in lesional skin or serum.

32. The method of claim 31, wherein the levels of one or more of microRNAs miR-223-5p and miR-223-3 in lesional skin or serum is correlated with GPPASI and/or GPPGA scores wherein treatment or prevention of flares of a subject with a history of GPP symptoms is measured as achieving one or more of the following results:
(a) maintenance of a Generalized Pustular Psoriasis Global Assessment (GPPGA) pustulation subscore of 0 or 1 and maintenance or decrease in the level of miR-223-5p and/or miR-223-3 after administering said anti-IL-36R antibody up week 48 after the initial loading dose;
(b) maintenance of a GPPGA total score of 0 or 1 and maintenance or decrease in the level of miR-223-5p and/or miR-223-3 after administration of said anti-IL-36R antibody up to week 48 after the initial loading dose;
(c) a sustained remission of GPP symptoms, defined as a subject treated with said anti-IL-36R antibody who maintains a GPPGA score of 0 or 1 (clear or almost clear) and maintenance or decrease in the level of miR-223-5p and/or miR-223-3 at all visits up to Week 48, without intake of GPP flare medication, or investigator-prescribed Standard of Care (SoC);
(d) a decrease in the occurrence of GPP flare(s) and maintenance or decrease in the level of miR-223-5p and/or miR-223-3, wherein GPP flare is defined as in increase in GPP Physician Global Assessment (GPPGA) total score of ≥2 and a GPPGA pustulation subscore of ≥2, from baseline up to week 48; or
(e) a prolongation of the time to a first GPP flare as measured by the time from baseline to the onset of said first GPP flare, wherein GPP flare is defined as in increase in the GPP Physician Global Assessment (GPPGA) total score of ≥2, and a GPPGA pustulation subscore of ≥2, and an increase in the level of miR-223-5p and/or miR-223-3 from baseline up to week 48.

33. The method of claim 29, wherein the levels of biomarkers are determined by small RNA sequencing, qPCR, or ELISA and IHC.

34. The method according to claim 29, further comprising continuing the administration of maintenance doses of the anti-IL-36R antibody to the patient if the difference in levels between the pre-treatment sample and the post-treatment reflects a beneficial response in the patient.

35. A method for determining whether a potential therapeutic agent is efficacious in the treatment and prevention of GPP comprising:
(a) obtaining a first biological sample from a GPP patient prior flare and/or prior to being treated with the potential therapeutic agent;
(b) treating the GPP patient with the potential therapeutic agent;
(c) obtaining a second biological sample from the GPP patient after being treated with the potential therapeutic agent;
(d) measuring in said first and second sample the levels of expression of one or more biomarkers; and
(e) comparing the biomarker levels in the second sample to the levels in the first sample,
wherein changes (e.g., lower or higher) in biomarker levels in the second sample than in the first sample indicate that the potential therapeutic agent is efficacious, and further wherein the one or more biomarkers comprise microRNAs selected from the group consisting of miR-223-3p, miR-223-5p, miR-1304-3p, miR-485-5p, or miR-337-5p.

36. The method according to claim 35, wherein changes (e.g., lower or higher) in biomarker levels in the second sample as compared to the first sample correlate with improvement in clinical efficacy measures.

37. The method according to claim 35, further comprising continuing the treatment of the patient if biomarker levels in the second sample change (e.g., are higher or lower) as compared to the first sample.

38. A method of treatment of generalized pustular psoriasis (GPP) in a subject with a history of GPP when not experiencing a flare comprising:
a) determining whether to initiate treatment of the subject, modify the treatment dose, modify the dosing interval, or discontinue treatment, based on the method of claim 29; and
b) modifying the treatment regimen based on the determination.

39. A method of monitoring patient response to a GPP treatment comprising:
(a) obtaining a first biological sample from the patient;
(b) measuring the level of one or more biomarkers in said first biological sample, wherein said one or more biomarkers comprise microRNAs selected from the group consisting of miR-223-3p, miR-223-5p, miR-1304-3p, miR-485-5p, or miR-337-5p;
(c) administering a treatment compound to the patient;
(d) obtaining a second biological sample from the patient;
(e) measuring the level of said one or more biomarkers in said second biological sample; and
(f) comparing the levels of the one or more biomarkers obtained from first and second biological samples;
wherein upregulation or downregulation post-treatment as compared to pre-treatment baseline indicates an effective response.

40. A method for monitoring patient compliance with a drug treatment protocol for the treatment and prevention of GPP flares comprising:
(a) obtaining a first biological sample from the patient;
(b) measuring the level of one or more biomarkers in said first biological sample, wherein said one or more biomarkers comprise microRNAs selected from the group consisting of miR-223-3p, miR-223-5p, miR-1304-3p, miR-485-5p, or miR-337-5p;
(c) administering a treatment compound to the patient;
(d) obtaining a second biological sample from the patient;
(e) measuring the level of said one or more biomarkers in said second biological sample; and
(f) comparing the levels of the one or more biomarkers obtained from first and second biological samples;
wherein upregulation or downregulation post-treatment as compared to pre-treatment baseline indicates patient compliance with said drug treatment protocol.

41. The method of claim 39, wherein the level of the one or more biomarkers in the second biological sample is decreased by at least about 20%, 30%, 40%, 50%, 55%, 60%, 65%, 65%, 70%, 75%, 80%, 85%, 90%, or 95% or more as compared to the level in the first biological sample.

42. The method of claim 39, wherein the biological sample is a skin biopsy, blood, plasma, or serum sample.

43. The method of claim 39, wherein the levels of biomarkers are determined by small RNA sequencing or ELISA and IHC.

44. The method of claim 39, wherein the treatment compound is an anti-IL-36R antibody.


SEQUENCE LISTING (142 sequences)

SEQ ID NO: 1 - protein, 108 aa, Mus sp.
QIVLTQSPAIMSASLGERVTMTCTASSSVSSSYLHWYQKKPGSSPKLWVYSTSNLASGVPVRFSGSGSGTSYSLTISSMEAEDAATYYCHQHHRSPVTFGSGTKLEMK

SEQ ID NO: 2 - protein, 107 aa, Mus sp.
DIQMTQSPASQSASLGESVTFTCLASQTIGTWLAWYQQRPGKSPQLLIYAATSLADGVPSRFSGSGSGTQFSFNIRSLQAEDFASYYCQQVYTTPLTFGGGTKLEIK

SEQ ID NO: 3 - protein, 107 aa, Mus sp.
DIQMTQSPASQSASLGESVTFTCLASQTIGTWLGWYQQKPGKSPQLLIYRSTTLADGVPSRFSGSGSGTKFSFKISSLQAADFASYYCQQLYSAPYTFGGGTKLEIR

SEQ ID NO: 4 - protein, 112 aa, Mus sp.
DVLLTQTPLSLPVSLGDQASISCRSSQNIVHSNGNTYLQWYLQKPGQSPKLLIYKVSNRFSGVPDRFSGSGSGTDFTLKISRVEAEDLGVYYCFQGSHVPFTFGAGTKLELK

SEQ ID NO: 5 - protein, 107 aa, synthetic construct
DIQMTQTTSSLSASLGDRVTISCRASQDIYKYLNWYQQKPDGTLKLLIYYTSGLHSGVPSRFSGSGSGTDFSLTISNLEPEDIATYFCQQDSKFPWTFGGDTKLEIK

SEQ ID NO: 6 - protein, 108 aa, Mus sp.
QIVLTQSPAIMSASLGERVTMTCTASSSVSSSYFHWYQQKPGSSPKLWIYRTSNLASGVPGRFSGSGSGTSYSLTISSMEAEDAATYYCHQFHRSPLTFGAGTKLELK

SEQ ID NO: 7 - protein, 107 aa, Mus sp.
DIVMTQSQKFLSTSVGVRVSVTCKASQDVGTNVLWYQQKIGQSPKPLIYSASYRHSGVPDRFTGSGSGTDFTLIISNVQSEDLAEYFCQQYSRYPLTFGPGTKLELK

SEQ ID NO: 8 - protein, 107 aa, Mus sp.
DIVMTQSQKFLSTSVGVRVSVTCKASQDVGTNVLWYQQKIGQSPKALIYSASYRHSGVPDRFTGSGSGTDFTLIITNVQSEDLAEYFCQQYSRYPLTFGPGTKLELK

SEQ ID NO: 9 - protein, 107 aa, Mus sp.
DIVMTQSQKFMSATVGGRVNITCKASQNVGRAVAWYQQKPGQSPKLLTHSASNRYTGVPDRFTGSGSGTDFTLTITNMQSEDLADYFCQQYSSYPLTFGAGTKLDLK

SEQ ID NO: 10 - protein, 107 aa, Mus sp.
DIQMTQSPASQSASLGESVTFSCLASQTIGTWLGWYQQKPGKSPQLLIYRATSLADGVPSRFSGSGSGTNFSFKISSLQAEDLASYYCQQLYSGPYTFGGGTKLEIR

SEQ ID NO: 11 - protein, 119 aa, Mus sp.
QVQLQQSGTELLKPGASVKLSCKASGNTVTSYWMHWVKQRPGQGLEWIGEILPSTGRTNYNENFKGKAMLTVDKSSSTAYMQLSSLASEDSAVYYCTIVYFGNPWFAYWGQGTLVTVSA

SEQ ID NO: 12 - protein, 125 aa, Mus sp.
EVQLQQSGPELVKPGASVKLSCKASGYTFTDNYMNWVRQSHGKSLEWIGRVNPSNGDTKYNQNFKGKATLTVDKSLSTAYMQLNGLTSEDSAVYYCGRTKNFYSSYSYDDAMDYWGQGTSVTVSS

SEQ ID NO: 13 - protein, 124 aa, Mus sp.
EVQLQQSGAEFVRPGASVKFSCTASGFNIKDDYIHWVRQRPEQGLEWVGRIDPANGNTKYAPKFQDKATITADTSSNTAYLQLSSLTSEDTAVYYCAKSFPNNYYSYDDAFAYWGQGTLVTVSA

SEQ ID NO: 14 - protein, 118 aa, Mus sp.
QVQLKESGPVLVAPSQSLSITCTVSGFSLTKFGVHWIRQTPGKGLEWLGVIWAGGPTNYNSALMSRLTISKDISQSQVFLRIDSLQTDDTAMYYCAKQIYYSTLVDYWGQGTSVTVSS

SEQ ID NO: 15 - protein, 120 aa, Mus sp.
QVQLKESGPGLVAPSQSLFITCTVSGFSLSSYEINWVRQVPGKGLEWLGVIWTGITTNYNSALISRLSISKDNSKSLVFLKMNSLQTDDTAIYYCARGTGTGFYYAMDYWGQGTSVTVSS

SEQ ID NO: 16 - protein, 119 aa, Mus sp.
QVQLQQPGADFVRPGASMRLSCKASGYSFTSSWIHWVKQRPGQGLEWIGEINPGNVRTNYNENFRNKATLTVDKSSTTAYMQLRSLTSADSAVYYCTVVFYGEPYFPYWGQGTLVTVSA

SEQ ID NO: 17 - protein, 119 aa, Mus sp.
QVQLKESGPGLVAPSQSLSITCTVSGFSLTNYAVHWVRQFPGKGLEWLGVIWSDGSTDFNAPFKSRLSINKDNSKSQVFFKMNSLQIDDTAIYYCARKGGYSGSWFAYWGQGTLVTVSA

SEQ ID NO: 18 - protein, 119 aa, Mus sp.
QVQLKESGPGLVAPSQSLSITCTVSGFSLTNYAVHWVRQFPGKGLEWLGVIWSDGSTDYNAPFKSRLSINKDNSKSQVFFKMNSLQTDDTAIYYCARKGGYSGSWFAYWGQGTLVTVSA

SEQ ID NO: 19 - protein, 117 aa, Mus sp.
QVQLKESGPVLVAPSQSLSITCTVSGFSLTNYGVHWVRQPPGKGLEWLGVIWPVGSTNYNSALMSRLSIHKDNSKSQVFLRMNSLQTDDTAIYYCAKMDWDDFFDYWGQGTTLTVSS

SEQ ID NO: 20 - protein, 124 aa, Mus sp.
EVQLQQSGAELVRPGASVRLSCTASGFNIKDDYIHWVRQRPKQGLEWLGRIDPANGNTKYDPRFQDKATITADTSSNTAYLHLSSLTSEDTAVYYCAKSFPDNYYSYDDAFAYWGQGTLVTVSA

SEQ ID NO: 21 - protein, 12 aa, Mus sp.
TASSSVSSSYLH

SEQ ID NO: 22 - protein, 11 aa, Mus sp.
LASQTIGTWLA

SEQ ID NO: 23 - protein, 11 aa, Mus sp.
LASQTIGTWLG

SEQ ID NO: 24 - protein, 16 aa, Mus sp.
RSSQNIVHSNGNTYLQ

SEQ ID NO: 25 - protein, 11 aa, Mus sp.
RASQDIYKYLN

SEQ ID NO: 26 - protein, 12 aa, Mus sp.
TASSSVSSSYFH

SEQ ID NO: 27 - protein, 11 aa, Mus sp.
KASQDVGTNVL

SEQ ID NO: 28 - protein, 11 aa, Mus sp.
KASQNVGRAVA

SEQ ID NO: 29 - protein, 11 aa, Mus sp.
LASQTIGTWLG

SEQ ID NO: 30 - protein, 7 aa, Mus sp.
STSNLAS

SEQ ID NO: 31 - protein, 7 aa, Mus sp.
AATSLAD

SEQ ID NO: 32 - protein, 7 aa, Mus sp.
RSTTLAD

SEQ ID NO: 33 - protein, 7 aa, Mus sp.
KVSNRFS

SEQ ID NO: 34 - protein, 7 aa, Mus sp.
YTSGLHS

SEQ ID NO: 35 - protein, 7 aa, Mus sp.
RTSNLAS

SEQ ID NO: 36 - protein, 7 aa, Mus sp.
SASYRHS

SEQ ID NO: 37 - protein, 7 aa, Mus sp.
SASNRYT

SEQ ID NO: 38 - protein, 7 aa, Mus sp.
RATSLAD

SEQ ID NO: 39 - protein, 9 aa, Mus sp.
HQHHRSPVT

SEQ ID NO: 40 - protein, 9 aa, synthetic construct
QQVYTTPLT

SEQ ID NO: 41 - protein, 9 aa, Mus sp.
QQLYSAPYT

SEQ ID NO: 42 - protein, 9 aa, Mus sp.
FQGSHVPFT

SEQ ID NO: 43 - protein, 9 aa, Mus sp.
QQDSKFPWT

SEQ ID NO: 44 - protein, 9 aa, Mus sp.
HQFHRSPLT

SEQ ID NO: 45 - protein, 9 aa, Mus sp.
QQYSRYPLT

SEQ ID NO: 46 - protein, 9 aa, Mus sp.
QQYSSYPLT

SEQ ID NO: 47 - protein, 9 aa, Mus sp.
QQLYSGPYT

SEQ ID NO: 48 - protein, 10 aa, Mus sp.
GNTVTSYWMH

SEQ ID NO: 49 - protein, 10 aa, Mus sp.
GYTFTDNYMN

SEQ ID NO: 50 - protein, 10 aa, Mus sp.
GFNIKDDYIH

SEQ ID NO: 51 - protein, 10 aa, Mus sp.
GFSLTKFGVH

SEQ ID NO: 52 - protein, 10 aa, Mus sp.
GFSLSSYEIN

SEQ ID NO: 53 - protein, 10 aa, Mus sp.
GYSFTSSWIH

SEQ ID NO: 54 - protein, 10 aa, Mus sp.
GFSLTNYAVH

SEQ ID NO: 55 - protein, 10 aa, Mus sp.
GFSLTNYGVH

SEQ ID NO: 56 - protein, 10 aa, Mus sp.
GFNIKDDYIH

SEQ ID NO: 57 - protein, 17 aa, Mus sp.
EILPSTGRTNYNENFKG

SEQ ID NO: 58 - protein, 17 aa, Mus sp.
RVNPSNGDTKYNQNFKG

SEQ ID NO: 59 - protein, 17 aa, Mus sp.
RIDPANGNTKYAPKFQD

SEQ ID NO: 60 - protein, 16 aa, Mus sp.
VIWAGGPTNYNSALMS

SEQ ID NO: 61 - protein, 16 aa, Mus sp.
VIWTGITTNYNSALIS

SEQ ID NO: 62 - protein, 15 aa, Mus sp.
EINPGNVRTNYNENF

SEQ ID NO: 63 - protein, 16 aa, Mus sp.
VIWSDGSTDFNAPFKS

SEQ ID NO: 64 - protein, 16 aa, Mus sp.
VIWSDGSTDYNAPFKS

SEQ ID NO: 65 - protein, 16 aa, Mus sp.
VIWPVGSTNYNSALMS

SEQ ID NO: 66 - protein, 17 aa, Mus sp.
RIDPANGNTKYDPRFQD

SEQ ID NO: 67 - protein, 10 aa, Mus sp.
VYFGNPWFAY

SEQ ID NO: 68 - protein, 16 aa, Mus sp.
TKNFYSSYSYDDAMDY

SEQ ID NO: 69 - protein, 15 aa, Mus sp.
SFPNNYYSYDDAFAY

SEQ ID NO: 70 - protein, 10 aa, Mus sp.
QIYYSTLVDY

SEQ ID NO: 71 - protein, 12 aa, Mus sp.
GTGTGFYYAMDY

SEQ ID NO: 72 - protein, 10 aa, Mus sp.
VFYGEPYFPY

SEQ ID NO: 73 - protein, 11 aa, Mus sp.
KGGYSGSWFAY

SEQ ID NO: 74 - protein, 9 aa, Mus sp.
MDWDDFFDY

SEQ ID NO: 75 - protein, 15 aa, Mus sp.
SFPDNYYSYDDAFAY

SEQ ID NO: 76 - protein, 108 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIK

SEQ ID NO: 77 - protein, 108 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIK

SEQ ID NO: 78 - protein, 108 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIK

SEQ ID NO: 79 - protein, 108 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIK

SEQ ID NO: 80 - protein, 108 aa, synthetic construct [sequence omitted]

SEQ ID NO: 81 - protein, 108 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIK

SEQ ID NO: 82 - protein, 108 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIK

SEQ ID NO: 83 - protein, 108 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIK

SEQ ID NO: 84 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIK

SEQ ID NO: 85 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIK

SEQ ID NO: 86 - protein, 107 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIK

SEQ ID NO: 87 - protein, 119 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSS

SEQ ID NO: 88 - protein, 119 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSS

SEQ ID NO: 89 - protein, 119 aa, synthetic construct [sequence omitted]

SEQ ID NO: 90 - protein, 119 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSS

SEQ ID NO: 91 - protein, 119 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSS

SEQ ID NO: 92 - protein, 119 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSS

SEQ ID NO: 93 - protein, 119 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSS

SEQ ID NO: 94 - protein, 119 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSS

SEQ ID NO: 95 - protein, 119 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSS

SEQ ID NO: 96 - protein, 119 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSS

SEQ ID NO: 97 - protein, 119 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSS

SEQ ID NO: 98 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSS

SEQ ID NO: 99 - protein, 119 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSS

SEQ ID NO: 100 - protein, 119 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSS

SEQ ID NO: 101 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSS

SEQ ID NO: 102 - protein, 7 aa, Mus sp.
RTSTLAS

SEQ ID NO: 103 - protein, 7 aa, Mus sp.
RTSILAS

SEQ ID NO: 104 - protein, 7 aa, Mus sp.
RTSRLAS

SEQ ID NO: 105 - protein, 7 aa, Mus sp.
RTSQLAS

SEQ ID NO: 106 - protein, 7 aa, Mus sp.
RTSKLAS

SEQ ID NO: 107 - protein, 10 aa, Mus sp.
GFSLTDYAVH

SEQ ID NO: 108 - protein, 15 aa, Mus sp.
EILPGVVRTNYNENF

SEQ ID NO: 109 - protein, 15 aa, Mus sp.
EINPGAVRTNYNENF

SEQ ID NO: 110 - protein, 15 aa, Mus sp.
EINPGLVRTNYNENF

SEQ ID NO: 111 - protein, 15 aa, Mus sp.
EINPGSVRTNYNENF

SEQ ID NO: 112 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, synthetic construct [sequence omitted]

SEQ ID NO: 114 - protein, 215 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 215 aa, synthetic construct
QIVLTQSPGTLSLSPGERATMTCTASSSVSSSYFHWYQQKPGQAPRLWIYRTSRLASGVPDRFSGSGSGTDFTLTISRLEPEDAATYYCHQFHRSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 215 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 215 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, synthetic construct
QVQLVQSGAEVKKPGASVKVSCKASGYSFTSSWIHWVKQAPGQGLEWMGEINPGNVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 7 aa, Mus sp.
RTSHLAS

SEQ ID NO: 141 - protein, 5 aa, Mus sp.
SSWIH

SEQ ID NO: 142 - protein, 17 aa, Mus sp.
EINPGNVRTNYNENFRN